(19) United States
(12) Patent Application Publication
(10) Pub. No.: US 2009/0214529 A9
(43) Pub. Date: Aug. 27, 2009

(54) KINESIN INHIBITORS

(75) Inventors: Ching-Cheng Wang, Taipei City (TW); Hung-Chuan Chen, Sijhih City (TW); Shu-Huei Wang, Taipei City (TW); Mei-Chun Lin, Banciao City (TW); Tien-Lan Shieh, Longtan Township (TW); Ying-Huey Huang, Changhua (TW); Shih-Chieh Chuang, Jhutang Township (TW); Chi-Hsin Richard King, Holladay, UT (US)

Correspondence Address: OCCHIUTI ROHLICEK & TSAO, LLP, 10 FAWCETT STREET, CAMBRIDGE, MA, 02138, US

(73) Assignee: TaiGen Biotechnology Co., Ltd., Taipei (TW)

(21) Appl. No.: 12/125,094

(22) Filed: May 22, 2008

Related U.S. Application Data
(60) Provisional application No. 60/931,291, filed on May 22, 2007.

(51) Int. Cl. A61K 33/24; C07D 239/70; C07D 253/10; A61K 31/495; A61K 31/53; A61K 31/439; A61K 31/4375; A61K 31/728; A61K 31/675; A61K 31/337; A61K 31/506; A61K 39/395; A61K 31/505; A61K 31/519; A61K 31/282; A61P 35/00; A61P 37/02

(52) U.S. Cl. 424/133.1; 544/250; 544/249; 544/251; 544/183; 514/243; 514/250; 514/267; 514/283; 514/274; 514/252.18; 514/255.05; 514/492; 424/649; 514/449; 514/54; 514/110; 424/178.1

(57) ABSTRACT

This invention relates to the compounds of formula (I) shown below. Each variable in formula (I) is defined in the specification. These compounds can be used to treat a kinesin Eg5 protein-mediated disorder.

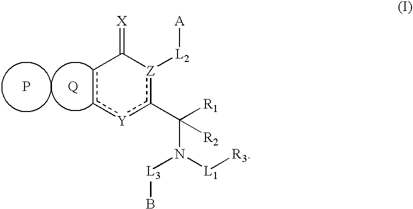

### CROSS REFERENCE TO RELATED APPLICATIONS

This application claims priority to U.S. Provisional Application Ser. No. 60/931,291, filed May 22, 2007. The contents of the prior application are hereby incorporated by reference in their entireties.

### BACKGROUND

Drugs that target mitosis are an important category of cancer therapeutics. Anti-mitotic drugs now being used in cancer clinics are typically tubulin binders, which block the cell division by interfering with normal assembly or disassembly of the mitotic spindle. See, e.g., Chabner, et al, “Antineoplastic agents” in Goodman and Gilman's “The Pharmacological Basis of Therapeutics,” 10th edition, 2001, MacGraw-Hill. For example, paclitaxel, one of the most effective anti-mitotic drugs, interferes with the growth and shrinkage of microtubules and blocks cells in the metaphase of mitosis, thereby resulting in cancer cell death. See, e.g., Blagosklonny, et al., Int. J. Cancer, 1999, 83:151-156.

Currently available anti-mitotic drugs typically exhibit side effects. For example, common side effects of paclitaxel include neutropenia and peripheral neuropathy. Moreover, some cancers are resistant to treatment with paclitaxel, and other cancers become insensitive during treatment. There still exists a need for developing anti-mitotic drugs that have fewer side effects and are more effective in treating cancers.

### SUMMARY

This invention is based on the unexpected discovery that certain tricyclic pyrimidinone derivatives are effective in inhibiting the activities of kinesin Eg5 proteins (KSPs) and therefore can be used to treat kinesin Eg5 protein-mediated disorders.

In one aspect, this invention features a compound of formula (I):

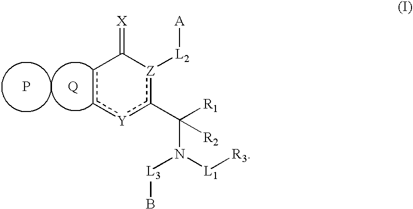

In the above formula,

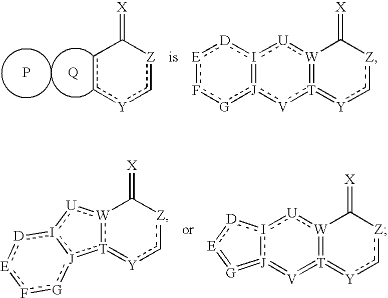

X is O or S; each of D, E, F, G, I, J, T, U, V, W, Y, and Z, independently, is C, C(Rₐ₁), C(Rₐ₁Rₐ₂); N,N(Rₐ₁), O, or S; each of Rₐ₁ and Rₐ₂, independently, being H, halo, CN, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, heteroaryl, COOR, OCOR, N(RR′), C(O)—N(RR′), or N(R)—C(O)R′; in which each of R and R′, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, or heteroaryl; each [symbol] independently, is a single bond or a double bond; each of A and B, independently, is aryl or heteroaryl; in which aryl or heteroaryl is optionally substituted by 1, 2, or 3 substituents selected from the group consisting of halo, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, heteroaryl, CN, NO₂, OR_{b1}, SR_{b1}, C(O)R_{b1}, COOR_{b1}, O(C)OR_{b1}, C(O)—N(R_{b1}R_{b2}), N(R_{b1})—C(O)R_{b2}, NR_{b1}R_{b2}, S(O)R_{b1}, S(O)₂R_{b1}, S(O)₂—NR_{b1}R_{b2}, NR_{b1}—S(O)₂R_{b2}, and C(NR_{b1})—NR_{b2}R_{b3}; each of R_{b1}, R_{b2}, and R_{b3}, independently, being H, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, or heteroaryl; each of R₁, R₂, and R₃, independently, is H, halo, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, heteroaryl, CN, NO₂, OR_{c1}, SR_{c1}, C(O)R_{c1}, COOR_{c1}, O(C)OR_{c1}, C(O)—N(R_{c1}R_{c2}), N(R_{c1})—C(O)R_{c2}, NR_{c1}R_{c2}, S(O)R_{c1}, S(O)₂R_{c1}, S(O)₂—NR_{c1}R_{c2}, NR_{c1}—S(O)₂R_{c2}, or C(NR_{c1})—NR_{c2}R_{c3}; or R₁ and R₂, together with the carbon atom to which they are attached, are C₃-C₂₀ cycloalkyl, C₃-C₂₀ cycloalkenyl, C₃-C₂₀ heterocycloalkyl, or C₃-C₂₀ heterocycloalkenyl; each of R_{c1}, R_{c2}, and R_{c3}, independently, being H, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, or heteroaryl; each of L₁ and L₂, independently, is O, N(R_{d1}), C₁-C₁₀ alkylene, C₁-C₁₀ alkylcycloalkylene, C₂-C₁₀ alkenylene, C₂-C₁₀ alkynylene, or deleted; R_{d1} being H or C₁-C₁₀ alkyl; and L₃ is CH₂, C(O), C(O)O, OC(O), SO, or SO₂.

Referring to formula (I) above, a subset of the compounds described above are those in which

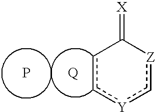

is selected from the group consisting of

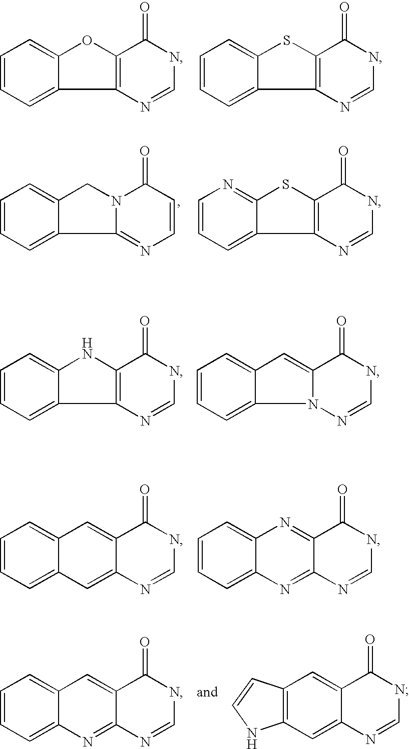

each of which is optionally substituted with F, Cl, Br, I, CN, COOR, OCOR, N(RR′), C(O)—N(RR′), N(R)—C(O)R′, or C₁-C₁₀ alkyl; the C₁-C₁₀ alkyl being optionally substituted with halo, C₂-C₁₀ alkenyl, or C₂-C₁₀ alkynyl. In these compounds, A can be one of phenyl, thienyl, and furanyl, each of which is optionally substituted with halo or C₁-C₁₀ alkyl; B can be phenyl optionally substituted with halo or C₁-C₁₀ alkyl; L₁ can be C₁-C₄ alkylene; L₂ can be C₁-C₃ alkylene; and L₃ can be C(O).

Referring to formula (I) above, another subset of the compounds described above are those in which R₁ is H; R₂ is H, halo, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, heteroaryl, CN, NO₂, OR_{c1}, SR_{c1}, C(O)R_{c1}, COOR_{c1}, O(C)OR_{c1}, C(O)—N(R_{c1}R_{c2}), N(R_{c1})—C(O)R_{c2}, NR_{c1}R_{c2}, S(O)R_{c1}, S(O)₂R_{c1}, S(O)₂—NR_{c1}R_{c2}, NR_{c1}—S(O)₂R_{c2}, or C(NR_{c1})—NR_{c2}R_{c3}; and the compound has a configuration as shown in the following formula:

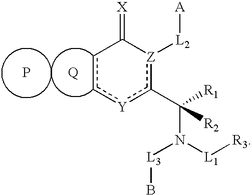

Some compounds of this subset feature that

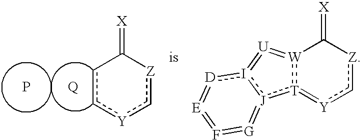

In these compounds, X can be O; Z can be C or N; Y can be N; W can be C, C(Rₐ₁), or N; T can be C, C(Rₐ₁), or N; U can be O, S, C(Rₐ₁), or C(Rₐ₁Rₐ₂); I can be C; D can be C(Rₐ₁) or N; E can be C(Rₐ₁); F can be C(Rₐ₁); G can be C(Rₐ₁); J can be C; L₁ can be C₂-C₄ alkylene or ethylcyclobutylene optionally substituted with OH, halo, or N(Rₑ₁Rₑ₂), in which each of Rₑ₁ and Rₑ₂, independently, can be H, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, or heteroaryl; L₂ can be methylene; and L₃ can be C(O) or SO₂; R₁ can be H; R₂ can be ethyl, n-propyl, isopropyl, isobutyl, cyclopropyl, cyclobutyl, cyclopentyl, or C(O)—N(R_{c1}R_{c2}); or R₁ and R₂, together with the carbon atom to which they are attached, can be C₃-C₂₀ cycloalkyl, C₃-C₂₀ cycloalkenyl, C₃-C₂₀ heterocycloalkyl, or C₃-C₂₀ heterocycloalkenyl; and R₃ can be one of N(R_{c1})—C(O)R_{c2}, NR_{c}R_{c2}, or NR_{c1}—S(O)₂R_{c2}; and each of A and B, independently, can be phenyl, naphthyl, pyridinyl, thienyl, furanyl, pyrrolyl, thiazolyl, isothiazolyl, oxazolyl, isooxazolyl, thiadiazolyl, oxadiazolyl, imidazolyl, and pyrazolyl, each of which is optionally substituted with 1, 2, or 3 substituents selected from the group consisting of F, Cl, Br, I, CN, NO₂, OR_{b1}, SR_{b1}, C₁-C₁₀ alkyl, C₂-C₁₀ alkenyl, C₂-C₁₀ alkynyl, C(O)R_{b1}, COOR_{b1}, O(C)OR_{b1}, C(O)—N(R_{b1}R_{b2}), N(R_{b1})—C(O)R_{b2}, NR_{b1}R_{b2}, S(O)R_{b1}, S(O)₂R_{b1}, S(O)₂—NR_{b1}R_{b2}, NR_{b1}—S(O)₂R_{b2}, and C(NR_{b1})—NR_{b2}R_{b3}. For example,

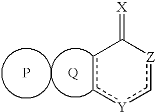

can be

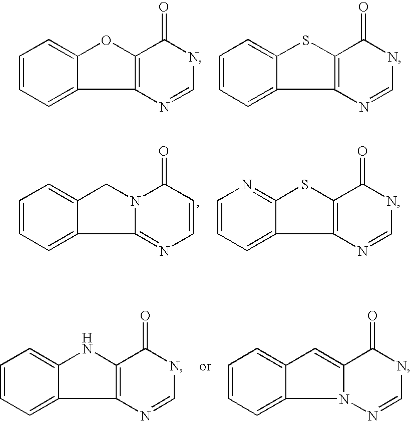

in which P is optionally substituted with F, Cl, Br, I, CN, COOR, OCOR, N(RR′), C(O)—N(RR′), N(R)—C(O)R′, or C₁-C₁₀ alkyl; the C₁-C₁₀ alkyl being optionally substituted with halo, C₂-C₁₀ alkenyl, or C₂-C₁₀ alkynyl.

Other compounds of this subset feature that

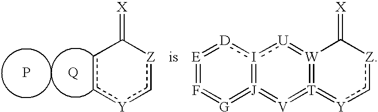

For example,

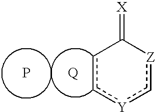

can be

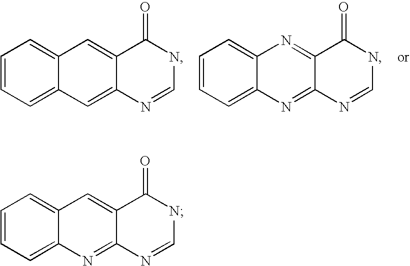

in which P is optionally substituted with Cl and Q is optionally substituted with Br, I, or CN. In these compounds, X can be O; Z can be N; Y can be N; W can be C; T can be C; U can be C(Rₐ₁) or N; V can be C(Rₐ₁) or N; I can be C; D can be C(Rₐ₁); E can be C(Rₐ₁); F can be C(Rₐ₁); G can be C(Rₐ₁); J can be C; L₁ can be C₂-C₄ alkylene optionally substituted with halo; L₂ can be methylene; L₃ can be CH₂ or C(O); R₁ can be H, R₂ can be ethyl or isopropyl; R₃ can be NH₂; A can be phenyl, or thienyl substituted with Cl; and B can be phenyl substituted with CH₃.

Still others compounds of this subset feature that

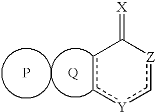

is

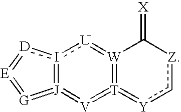

For example, it can be

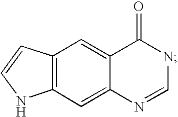

in which P is optionally substituted with Br. In these compounds, X can be O; Z can be N; Y can be N; W can be C; T can be C; U can be C(Rₐ₁); V can be C(Rₐ₁); I can be C; D can be C(Rₐ₁); E can be C(Rₐ₁); G can be N(Rₐ₁); J can be C; L₁ can be propylene; L₂ can be methylene; L₃ can be C(O); R₁ can be H, R₂ can be isopropyl; R₃ can be NH₂; A can be thienyl; and B can be phenyl substituted with CH₃.

The term “alkyl” refers to a saturated, linear or branched hydrocarbon moiety, such as —CH₃ or —CH(CH₃)₂. The term “alkylene” refers to a divalent, saturated, linear or branched hydrocarbon moiety, such as —CH₂— or —CH₂—CH(CH₃)—. The term “alkenyl” refers to a linear or branched hydrocarbon moiety that contains at least one double bond, such as —CH═CH—CH₃. The term “alkenylene” refers to a divalent, linear or branched hydrocarbon moiety containing a double bond, such as —CH═CH— or —CH═C(CH₃)—. The term “alkynyl” refers to a linear or branched hydrocarbon moiety that contains at least one triple bond, such as —C≡C—CH₃. The term “alkynylene” refers to a divalent, linear or branched hydrocarbon moiety containing a triple bond, such as —C≡C— or —CH(CH₃)—C≡C—. The term “cycloalkyl” refers to a saturated, cyclic hydrocarbon moiety, such as a cyclopropyl. The term “alkylcycloalkylene” refers to a divalent, saturated, hydrocarbon moiety containing a cycloalkyl group substituted with an alkyl group in which one radical is located at an alkyl moiety and the other radical is located at the cycloalkyl moiety. An example of alkylcycloalkylene is

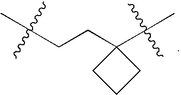

The term “cycloalkenyl” refers to a non-aromatic, cyclic hydrocarbon moiety that contains at least one ring double bond, such as cyclohexenyl. The term “heterocycloalkyl” refers to a saturated, cyclic moiety having at least one ring heteroatom (e.g., N, O, or S), such as 4-tetrahydropyranyl. The term “heterocycloalkenyl” refers to a non-aromatic, cyclic moiety having at least one ring heteroatom (e.g., N, O, or S) and at least one ring double bond, such as pyranyl. The term “aryl” refers to a hydrocarbon moiety having one or more aromatic rings. Examples of aryl moieties include phenyl (Ph), naphthyl, pyrenyl, fluorenyl, anthryl, and phenanthryl. The term “heteroaryl” refers to a moiety having one or more aromatic rings that contain at least one ring heteroatom (e.g., N, O, or S). Examples of heteroaryl moieties include furyl, pyrrolyl, thienyl, oxazolyl, imidazolyl, thiazolyl, pyridyl, pyrimidinyl, quinazolinyl, quinolyl, isoquinolyl, and indolyl.

Alkyl, alkylene, alkenyl, alkenylene, alkynyl, alkynylene, cycloalkyl, cycloalkenyl, alkylcycloalkylene, heterocycloalkyl, heterocycloalkenyl, aryl, and heteroaryl mentioned herein include both substituted and unsubstituted moieties, unless specified otherwise. Possible substituents on cycloalkyl, cycloalkenyl, alkylcycloalkylene, heterocycloalkyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, nitroso, azido, acyl, thioacyl, acyloxy, carboxyl, and carboxylic ester. On the other hand, possible substituents on alkyl, alkylene, alkenyl, alkenylene, alkynyl, and alkynylene include all of the above-recited substituents except C₁-C₁₀ alkyl. Cycloalkyl, cycloalkenyl, alkylcycloalkylene, heterocycloalkyl, heterocycloalkenyl, aryl, and heteroaryl can also be fused with each other.

The compounds described above contain asymmetric centers. Thus, they can occur as racemates and racemic mixtures, single enantiomers, individual diastereomers, diastereomeric mixtures, and cis- or trans-isomeric forms. All such isomeric forms are contemplated.

The compounds described above include the compounds themselves, as well as their salts, prodrugs, and solvates, if applicable. A salt, for example, can be formed between an anion and a positively charged group (e.g., amino) on a compound of this invention. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a compound of this invention. Suitable cations include sodium ion, potassium ion, magnesium ion, calcium ion, and an ammonium cation such as tetramethylammonium ion. The compounds of this invention also include those salts containing quaternary nitrogen atoms. Examples of prodrugs include esters and other pharmaceutically acceptable derivatives, which, upon administration to a subject, are capable of providing active compounds of this invention. A solvate refers to a complex formed between an active compound of this invention and a pharmaceutically acceptable solvent. Examples of pharmaceutically acceptable solvents include water, ethanol, isopropanol, ethyl acetate, acetic acid, and ethanolamine.

In another aspect, this invention features a pharmaceutical composition that contains an effective amount of at least one of the above-mentioned compounds and a pharmaceutically acceptable carrier.

In still another aspect, this invention features a method for treating a kinesin Eg5 protein-mediated disorder. The method includes administering to a subject in need thereof an effective amount of one or more of the compounds described above. The term “treating” or “treatment” refers to administering one or more of the compounds described above to a subject, who has an above-described disorder, a symptom of such a disorder, or a predisposition toward such a disorder, with the purpose to confer a therapeutic effect, e.g., to cure, relieve, alter, affect, ameliorate, or prevent the above-described disorder, the symptom of it, or the predisposition toward it. Examples of kinesin Eg5 protein-mediated disorders include cancer (e.g., Hodgkin's disease, multiple myeloma, lymphoma, hematological neoplasm, leukemia, non-small-cell lung cancer, renal cell carcinoma, hepatocellular carcinoma, melanoma, prostate cancer, pancreatic cancer, gastric cancer, esophageal cancer, bladder cancer, breast cancer, head and neck cancer, ovarian cancer, or colorectal cancer), hyperplasia, inflammation, immune disorder, restenosis, and cardiac hypertrophy. For example, a kinesin Eg5 protein-mediated disorder can be solid cancer.

A subject in need of treatment of an above-described disease can also be concurrently administered with a compound described above and one or more other anti-cancer agents. Examples of such anti-cancer agents include irinotecan, topotecan, gemcitabin, imatinib, trastuzuamb, 5-fluorouracil, leucovorin, carboplatin, cisplatin, docetaxel, paclitaxel, capecitabine, tezacitabine, cyclophosphamide, vinca alkaloid, anthracyclines, rituximab, and trastuzumab. The term “concurrently administered” refers to administering a compound described above and one or more other therapeutic agents at the same time or at different times during the period of treatment.

Also within the scope of this invention is a composition containing one or more of the compounds described above for use in treating an above-described disorder, and the use of such a composition for the manufacture of a medicament for the just-mentioned treatment.

The details of one or more embodiments of the invention are set forth in the description below. Other features, objects, and advantages of the invention will be apparent from the description and the claims.

### DETAILED DESCRIPTION

Shown below are exemplary compounds, compounds 1-226, of this invention:

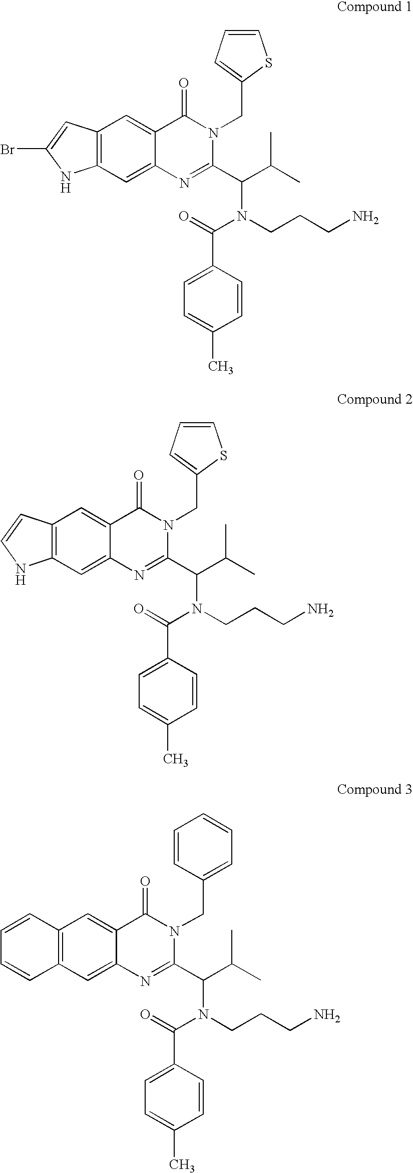

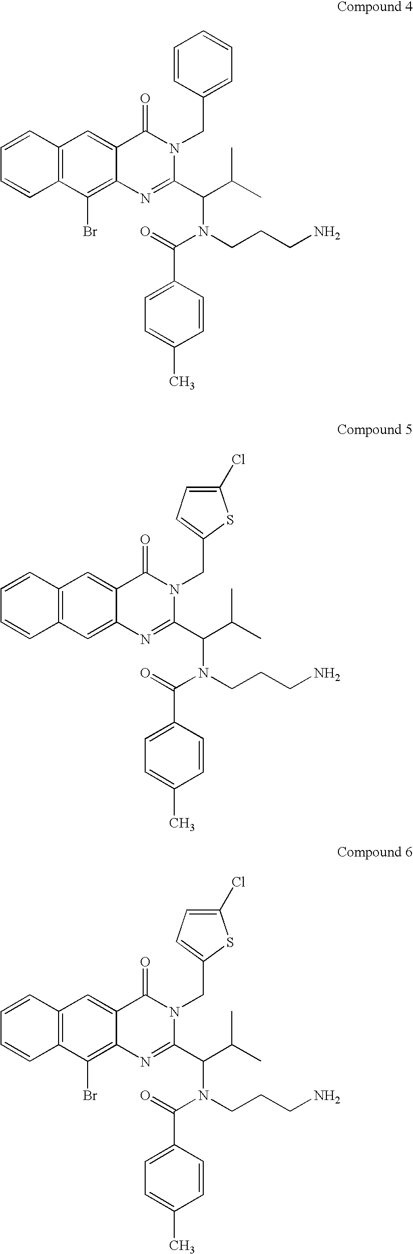

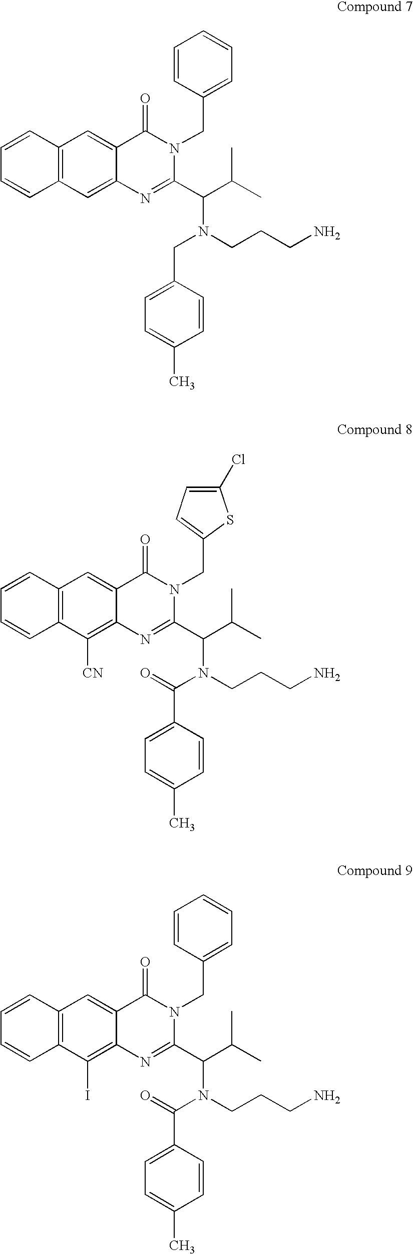

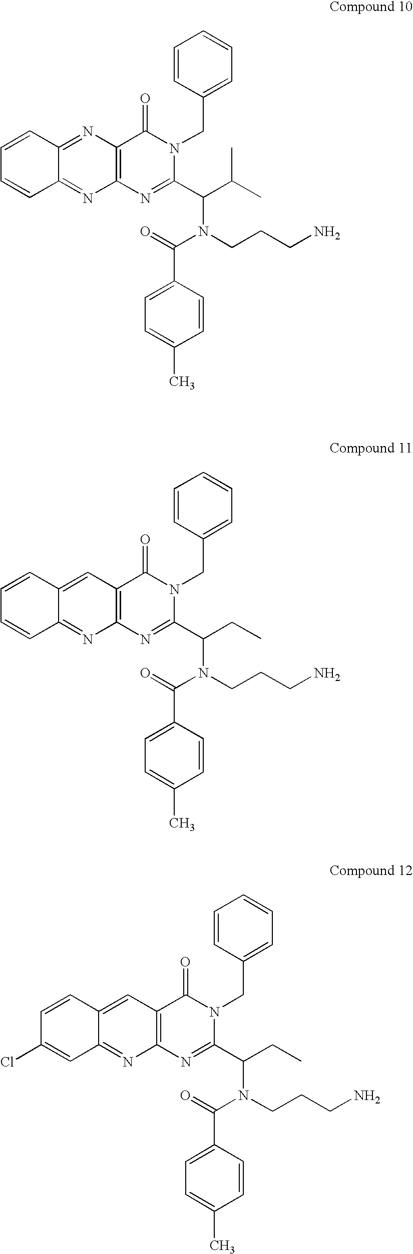

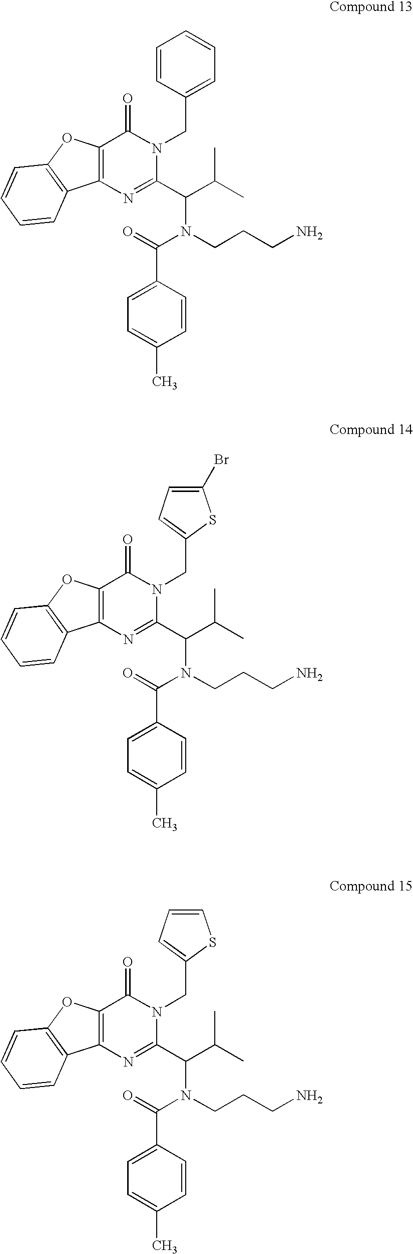

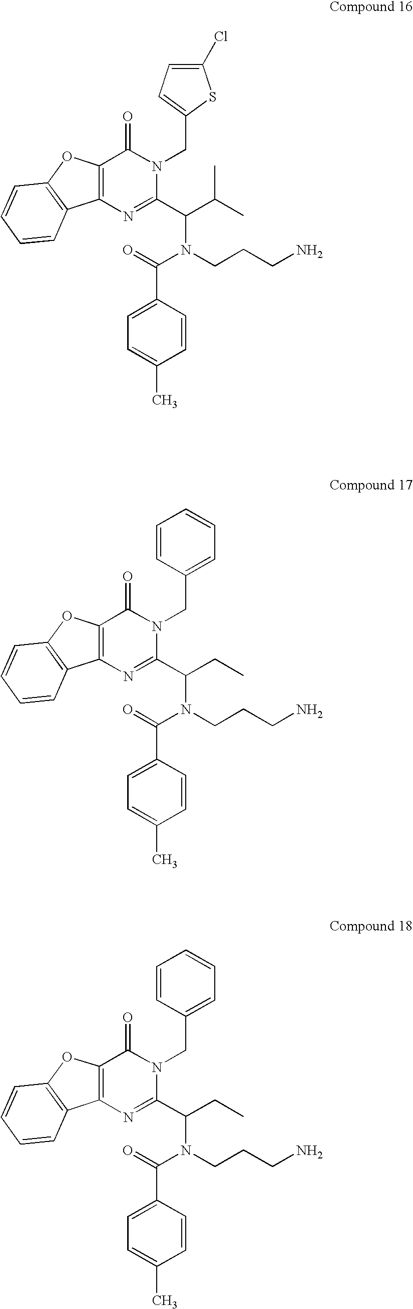

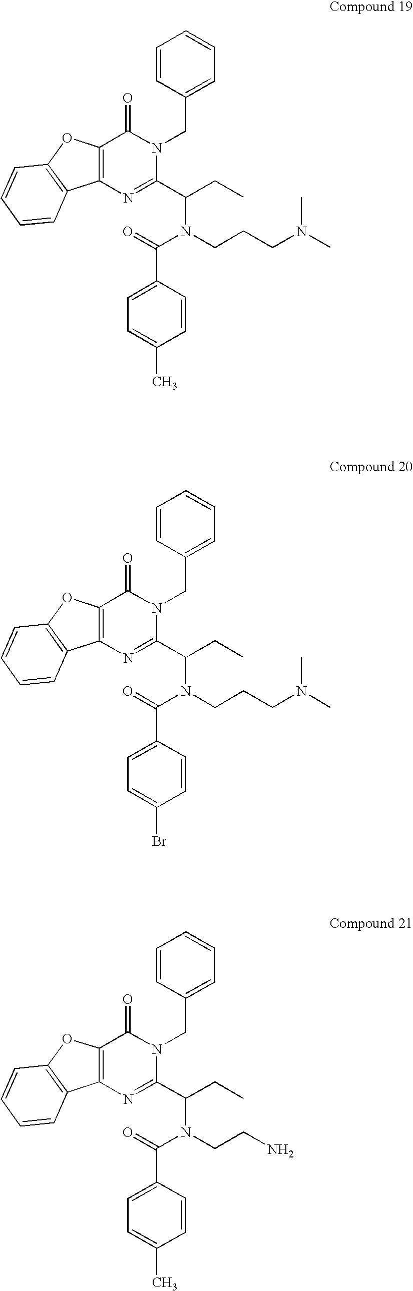

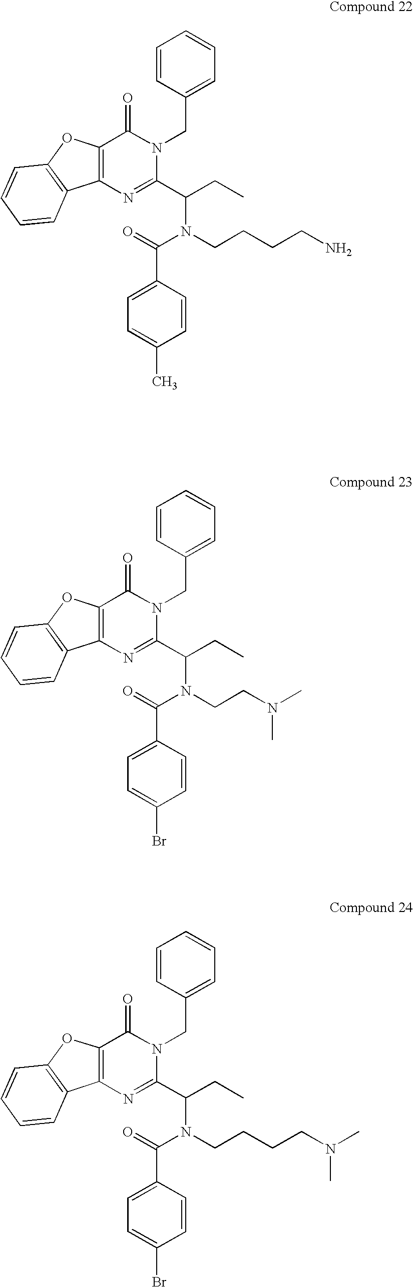

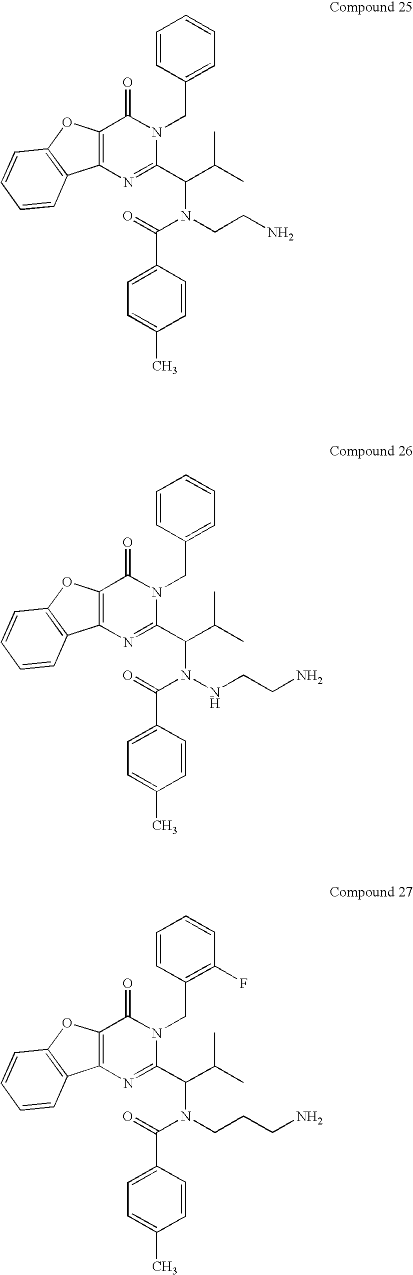

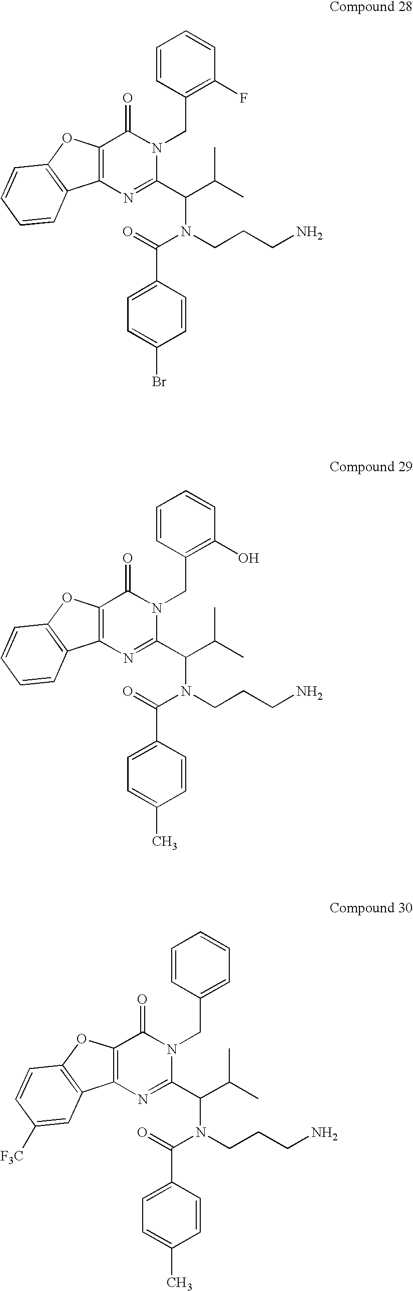

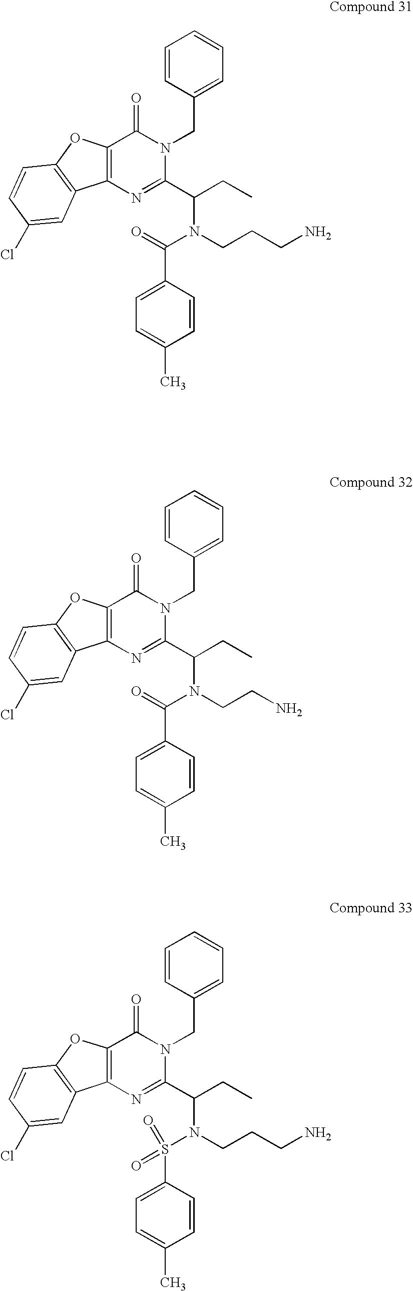

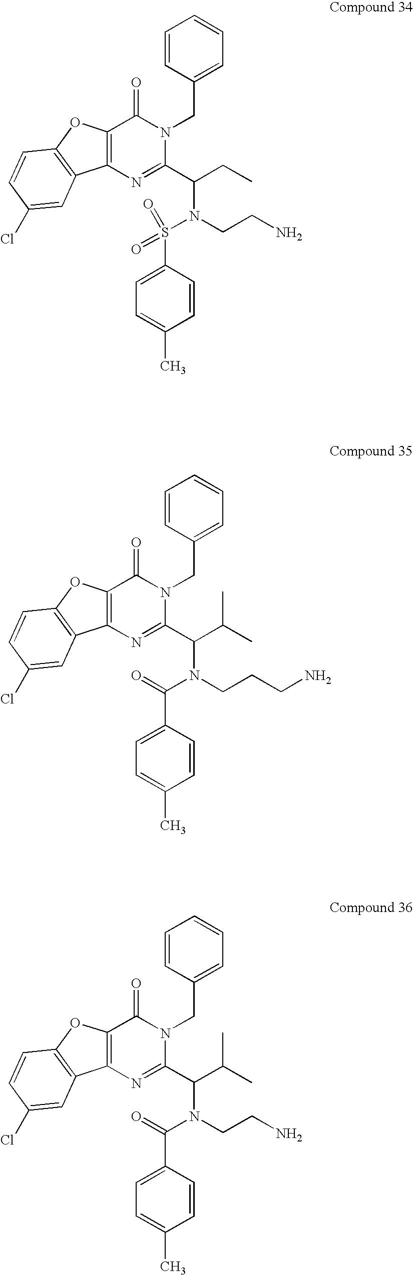

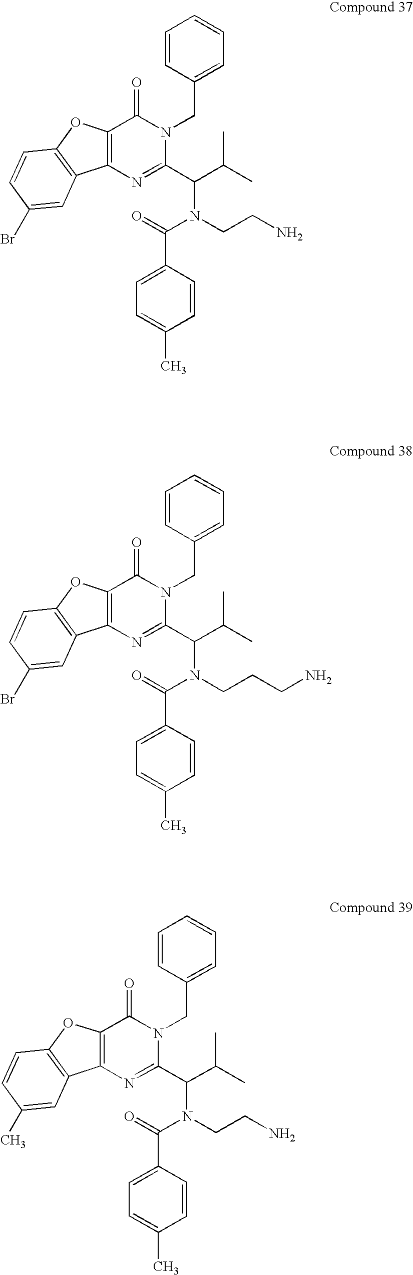

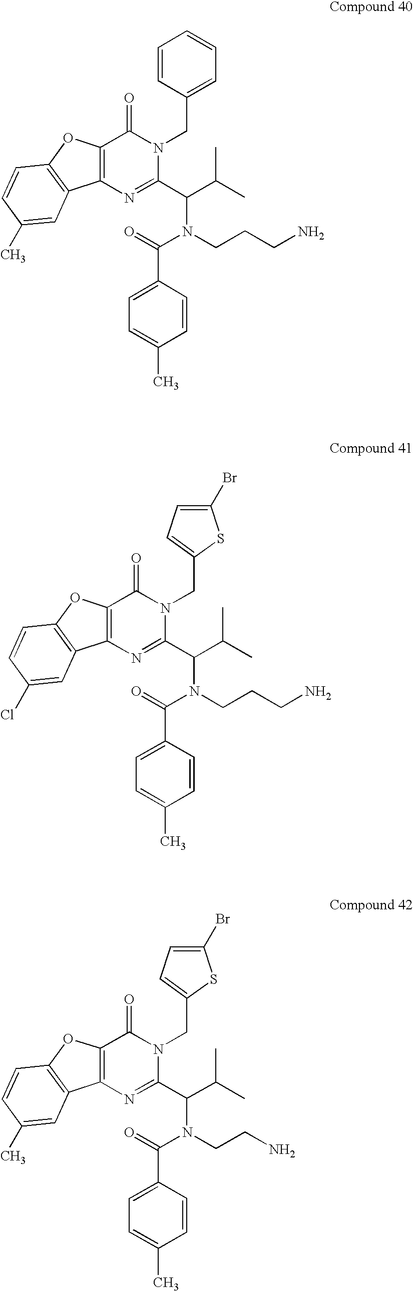

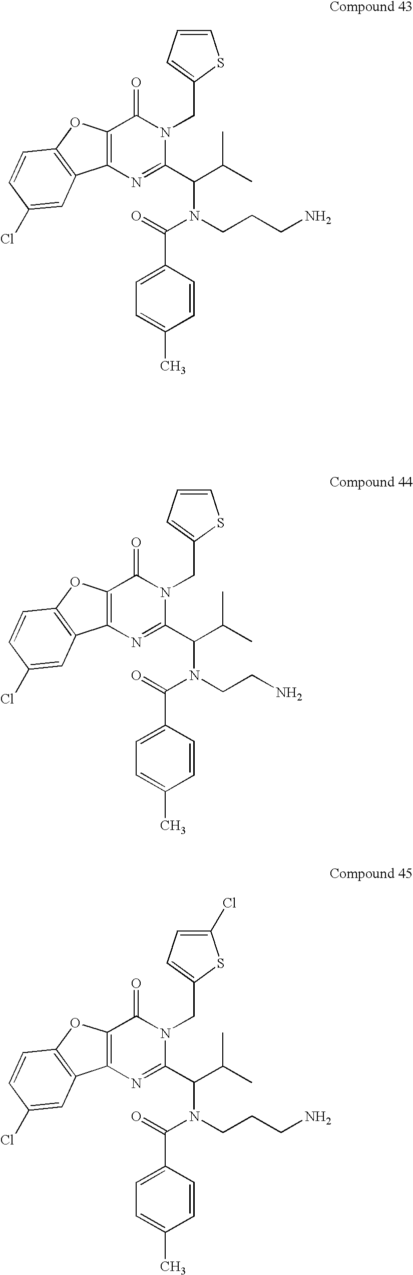

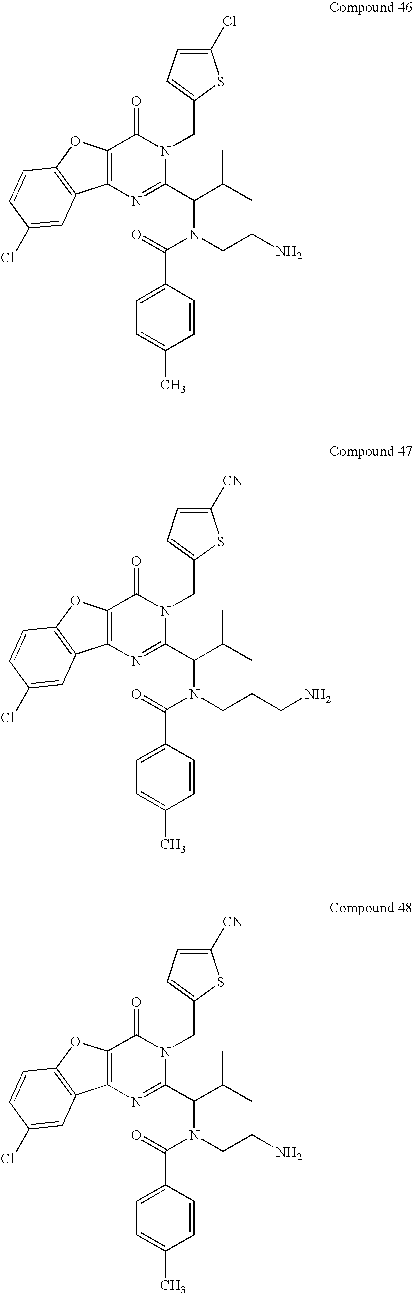

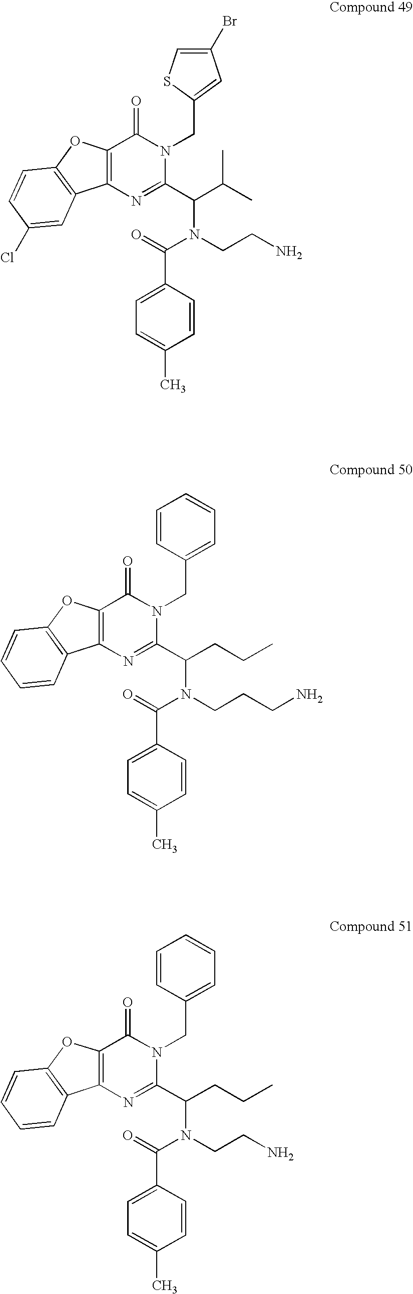

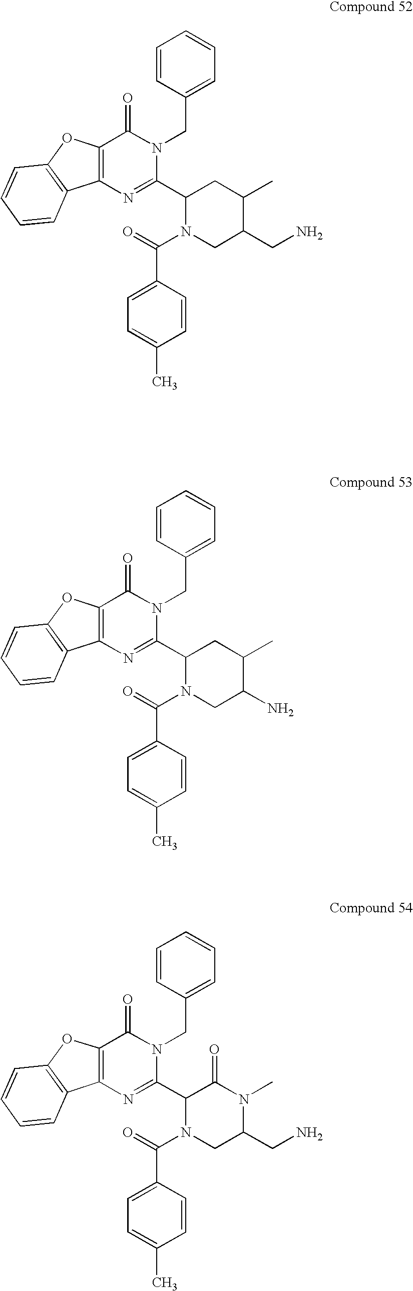

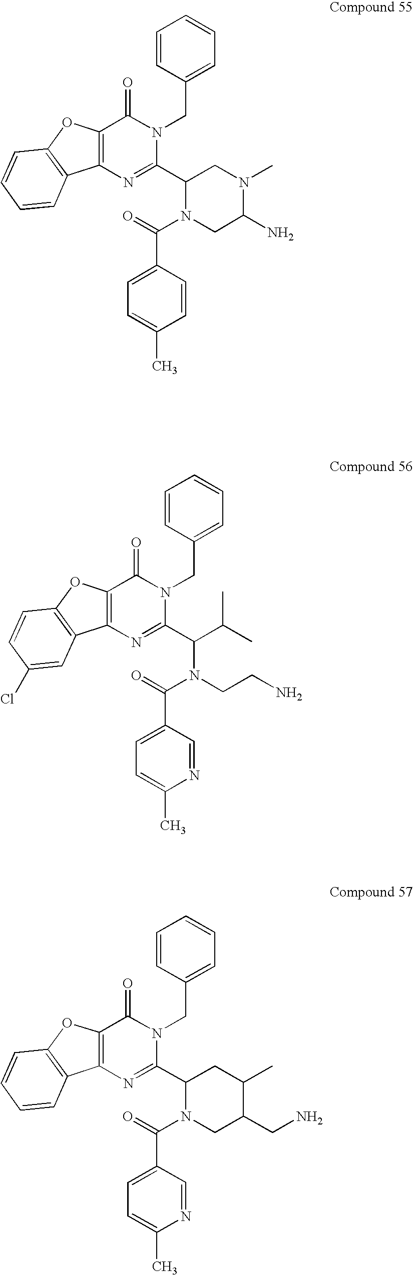

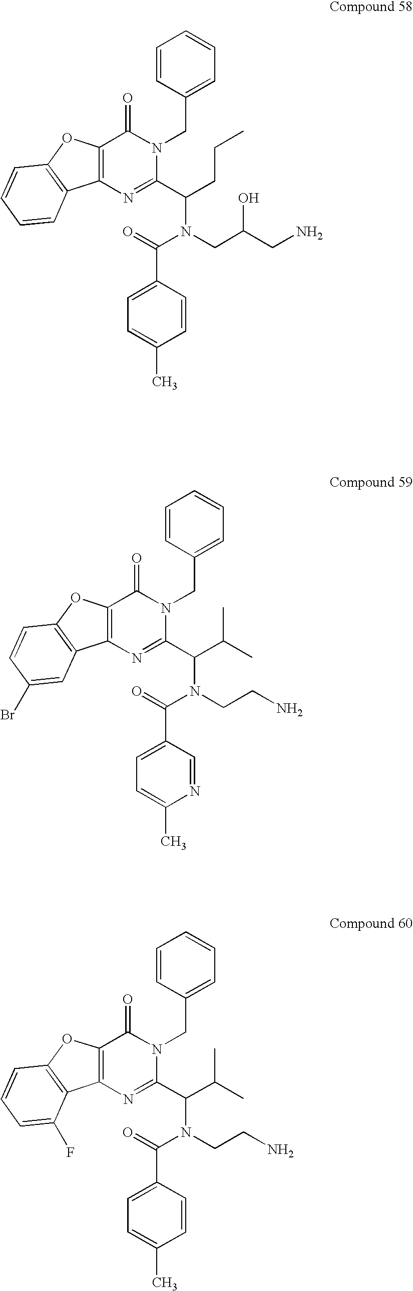

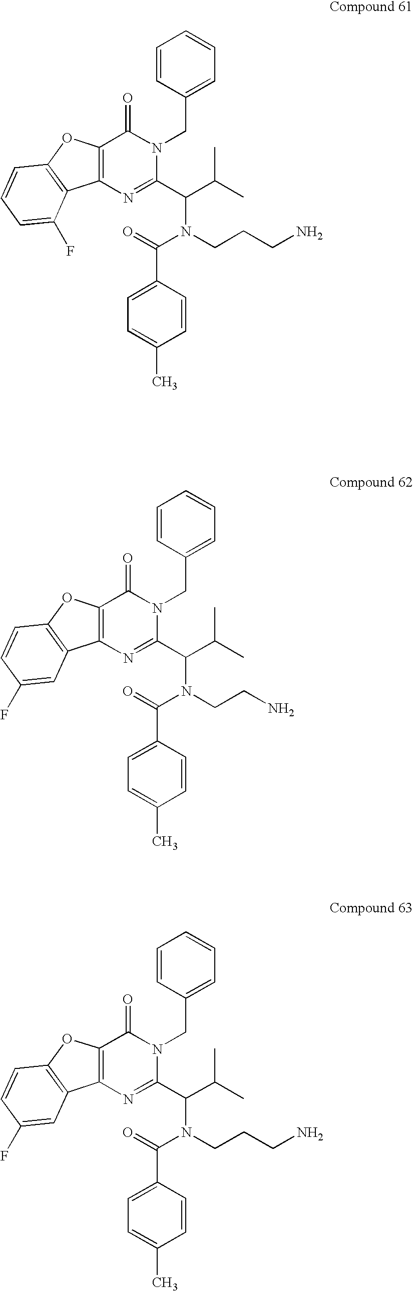

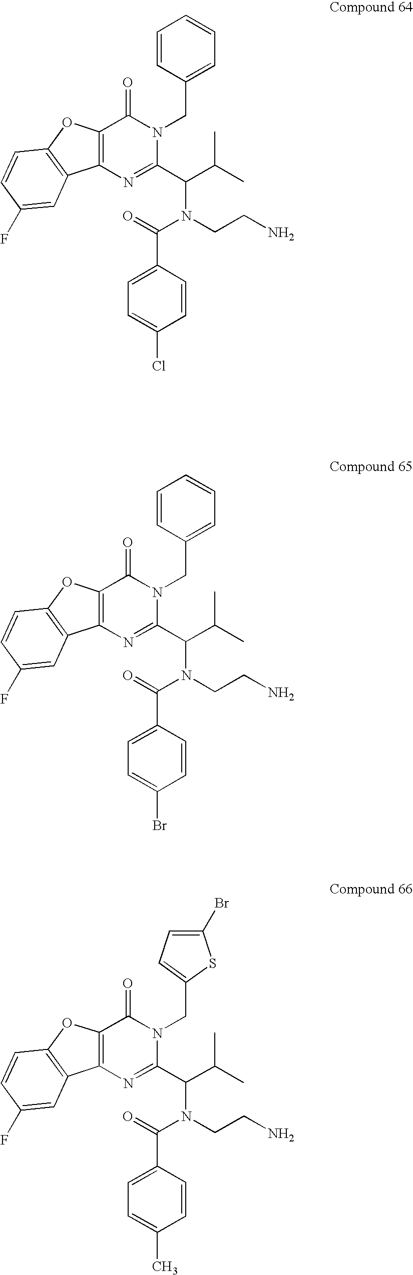

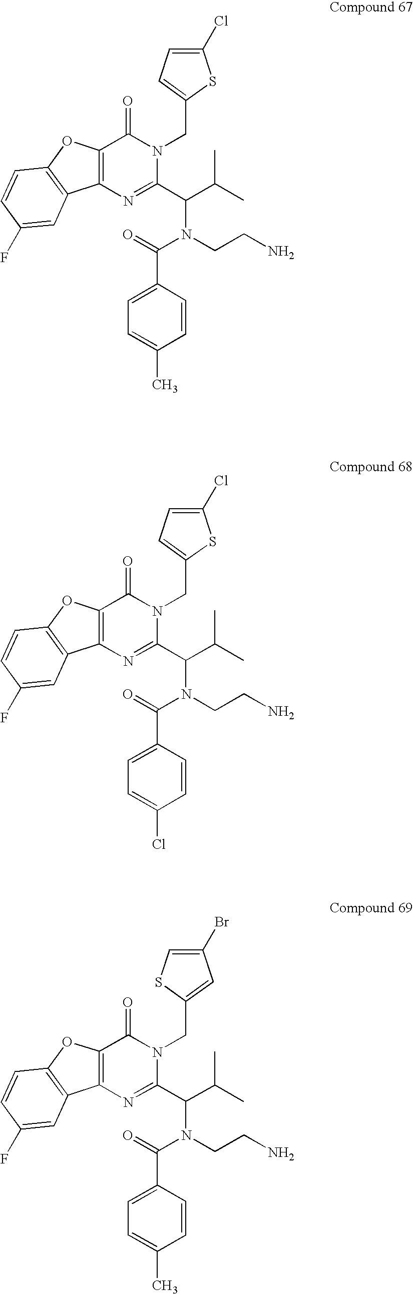

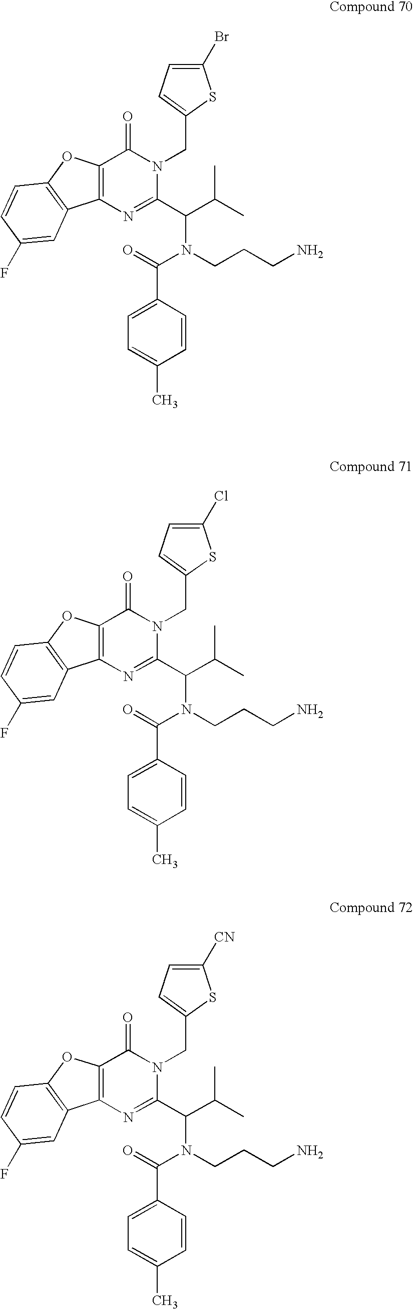

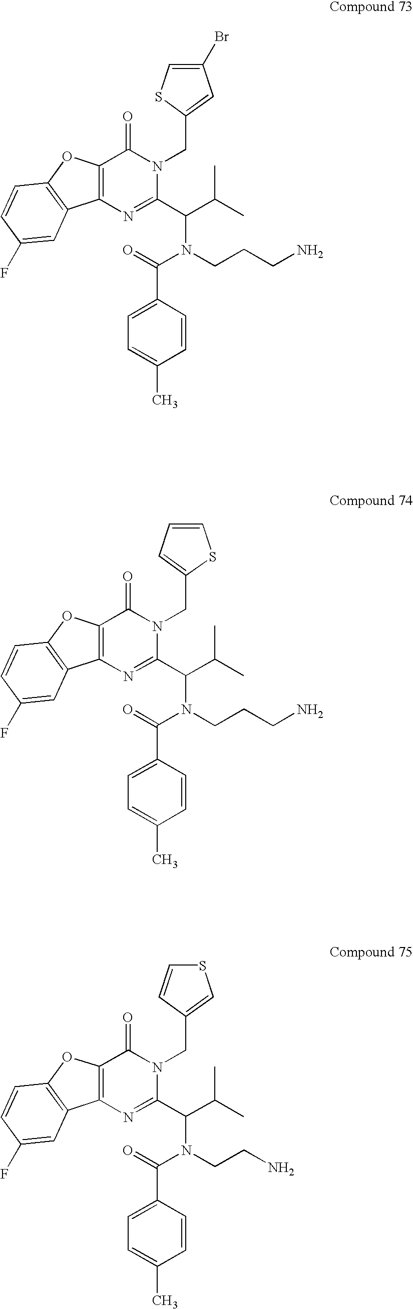

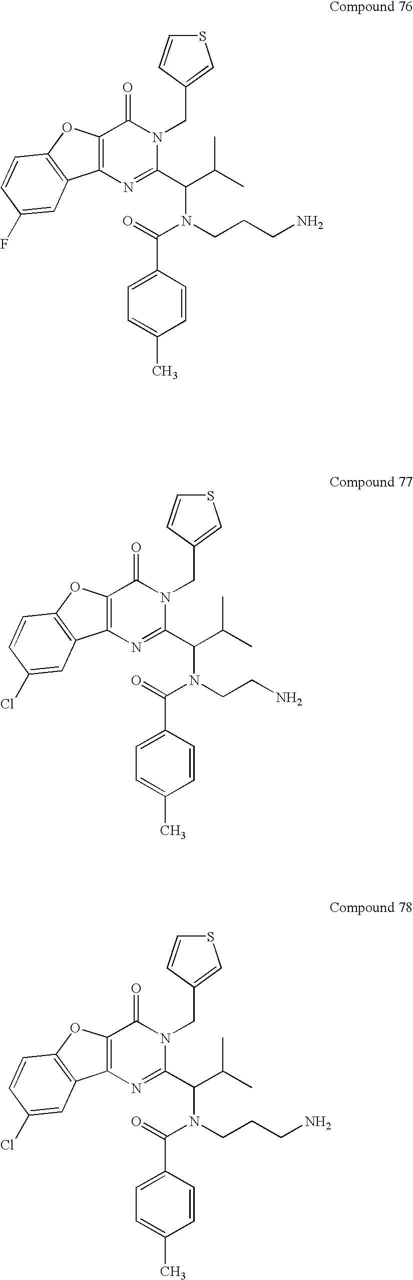

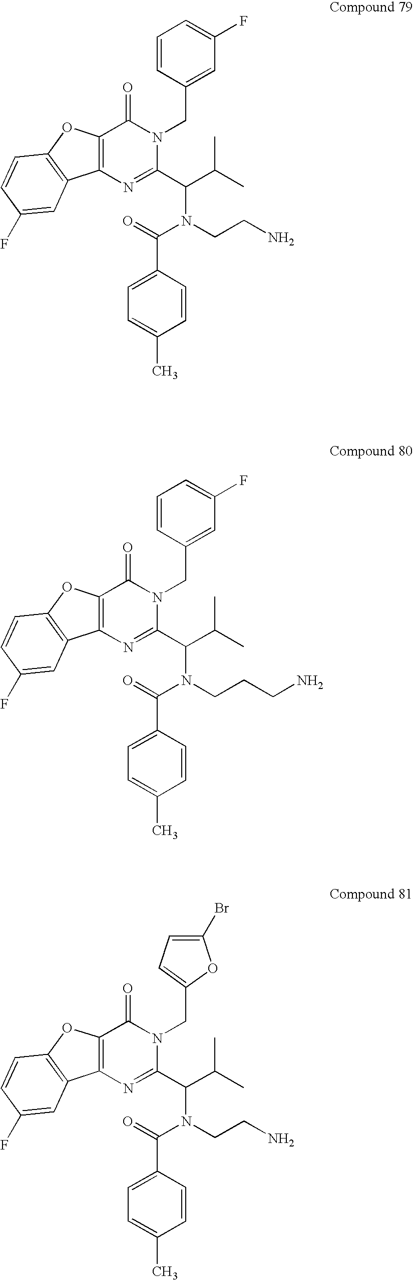

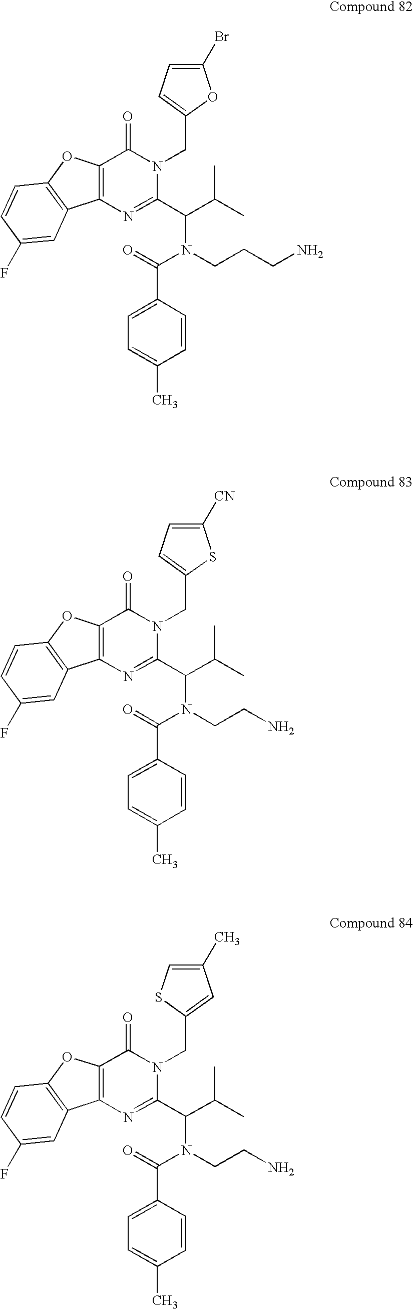

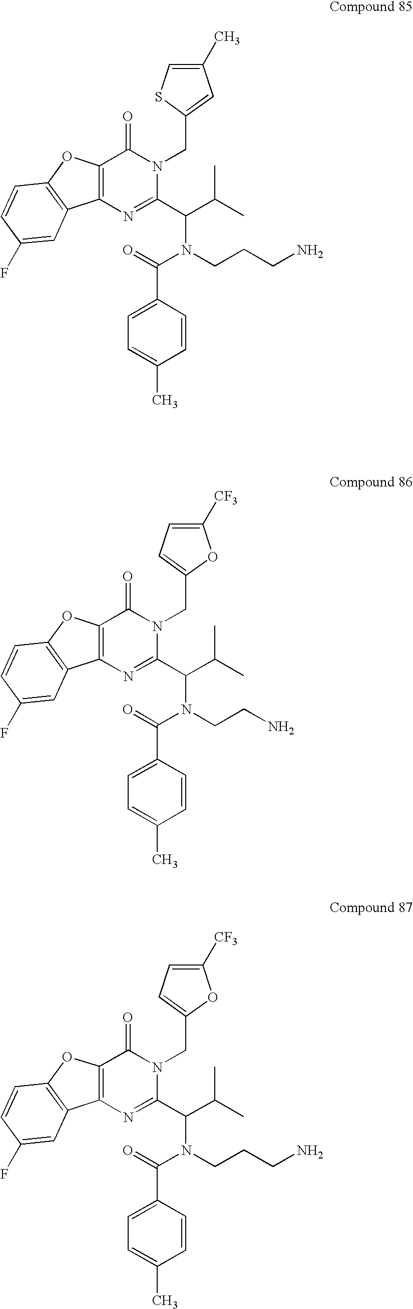

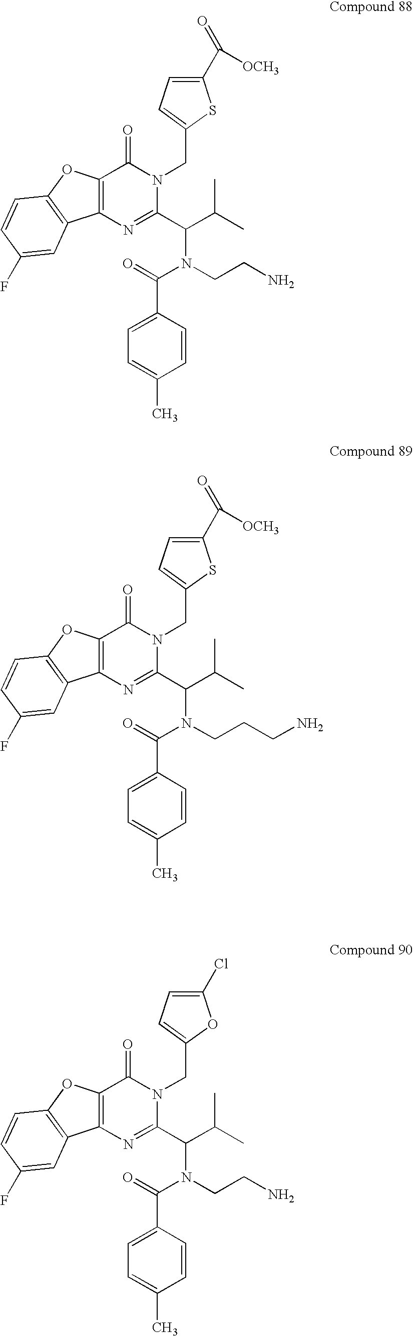

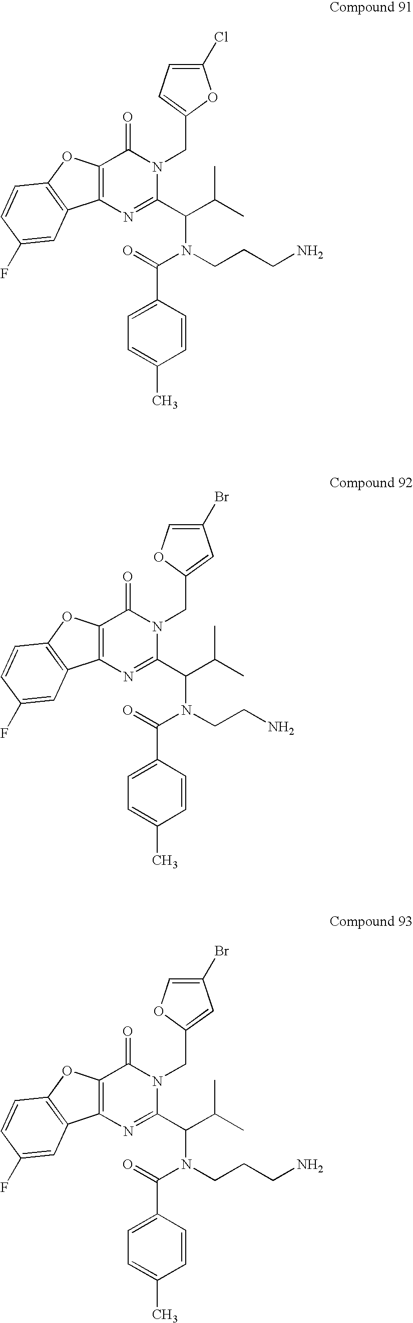

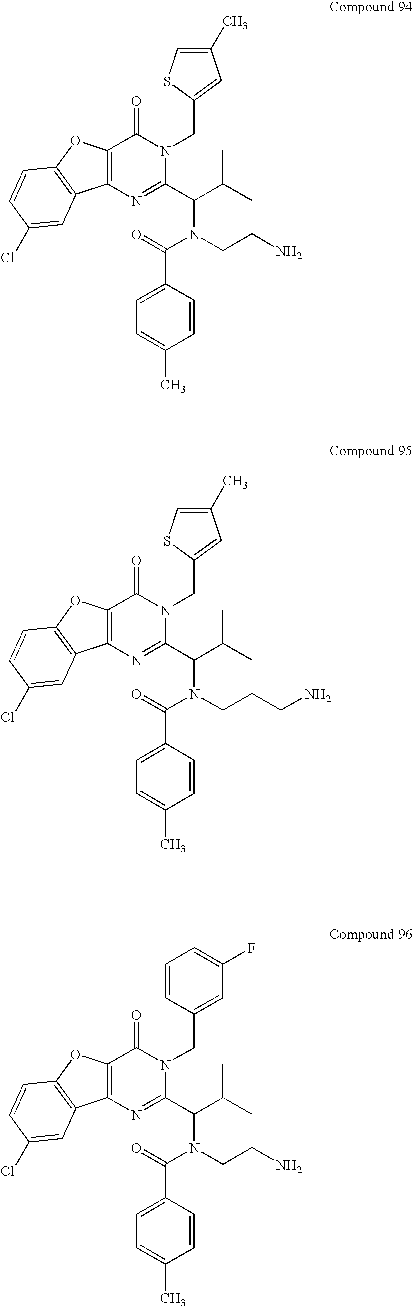

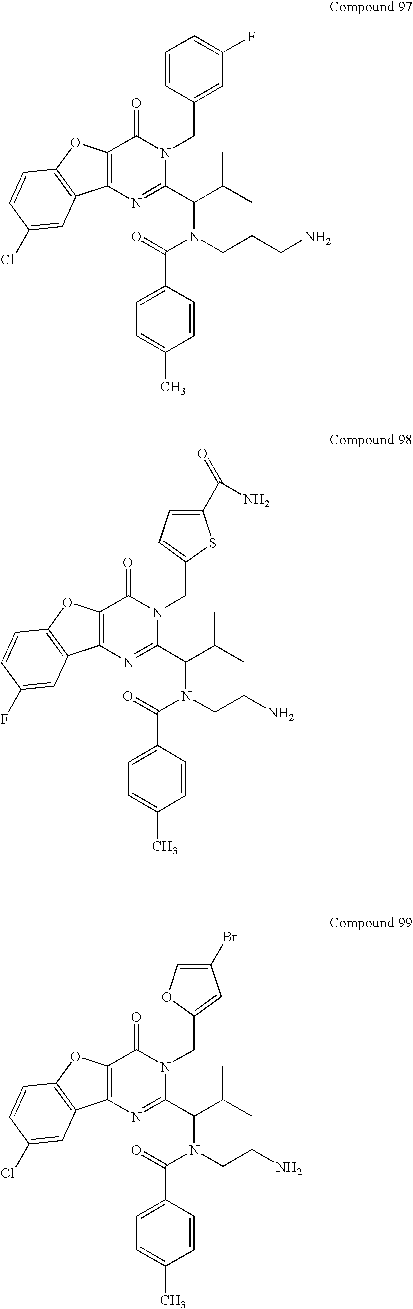

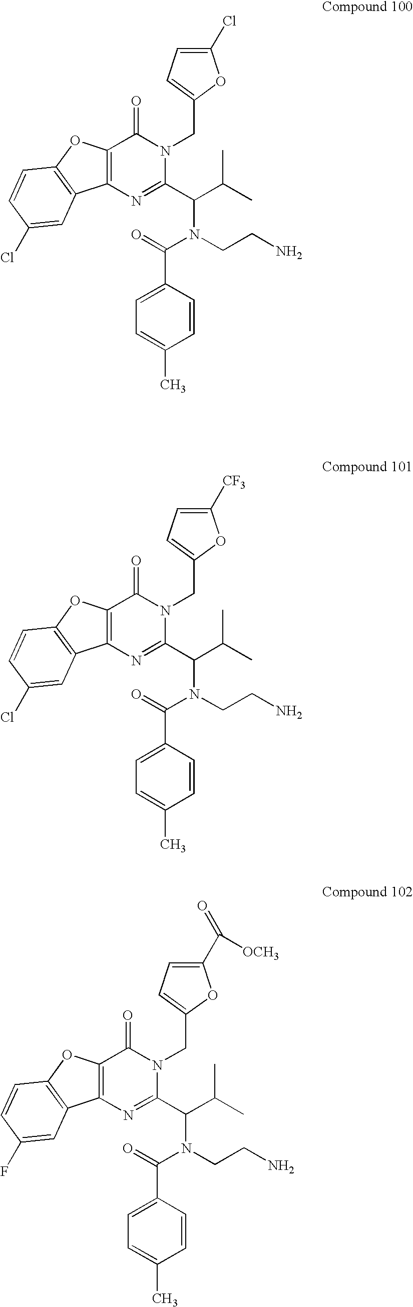

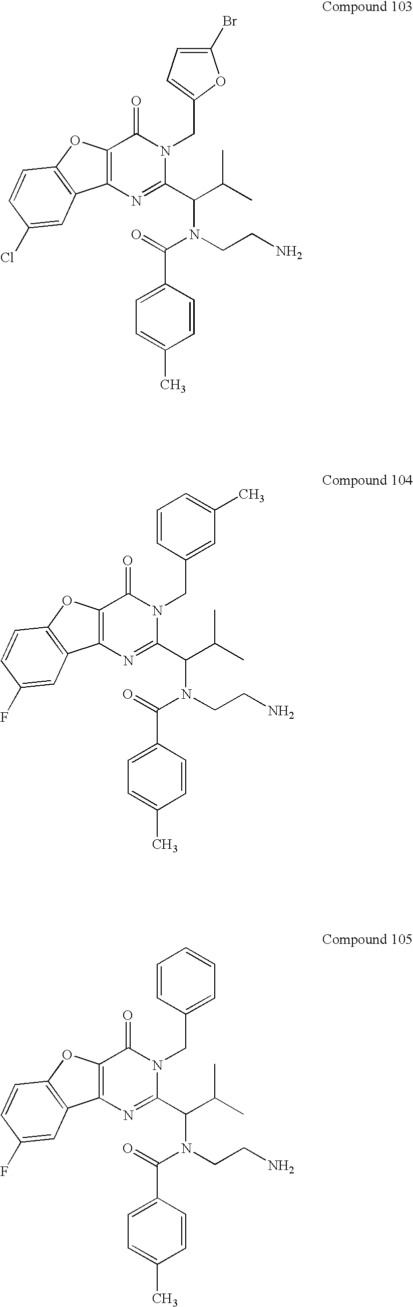

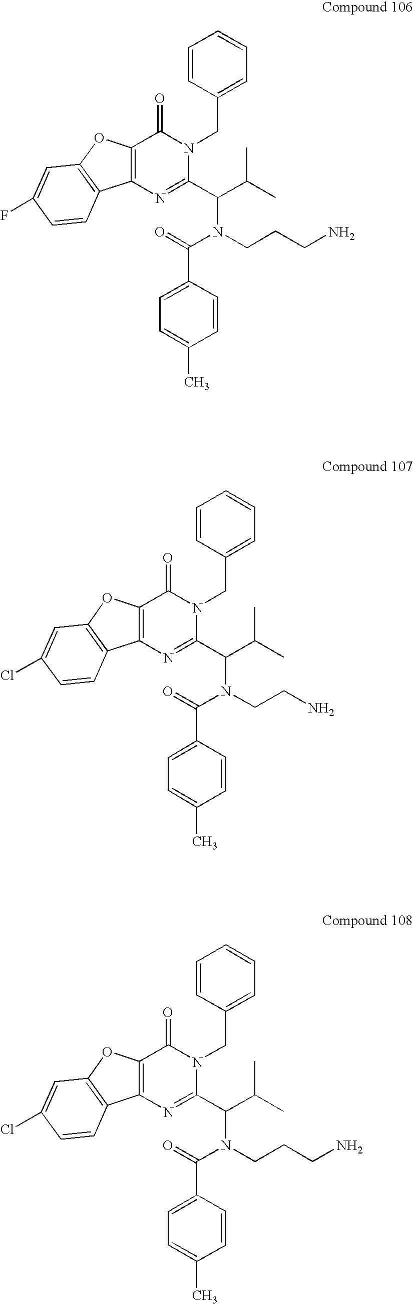

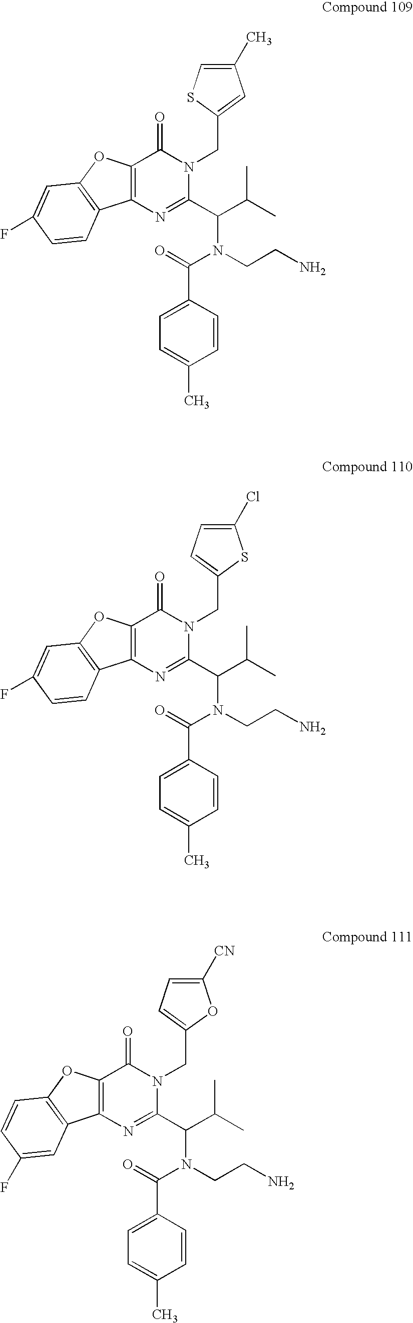

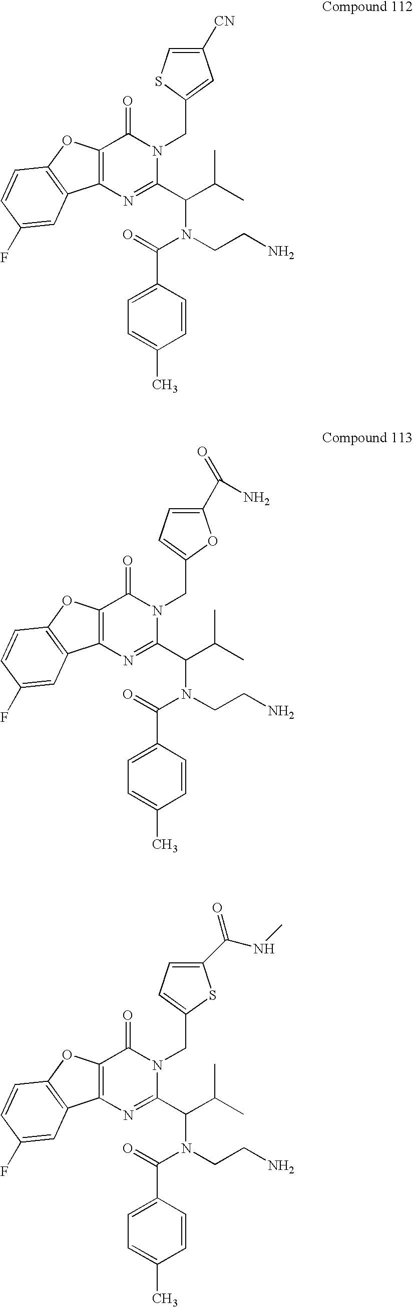

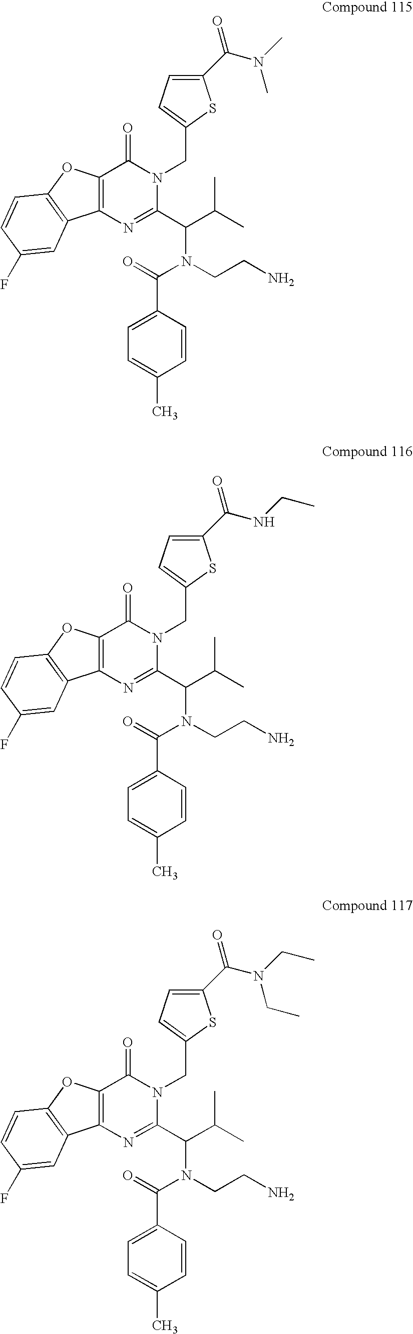

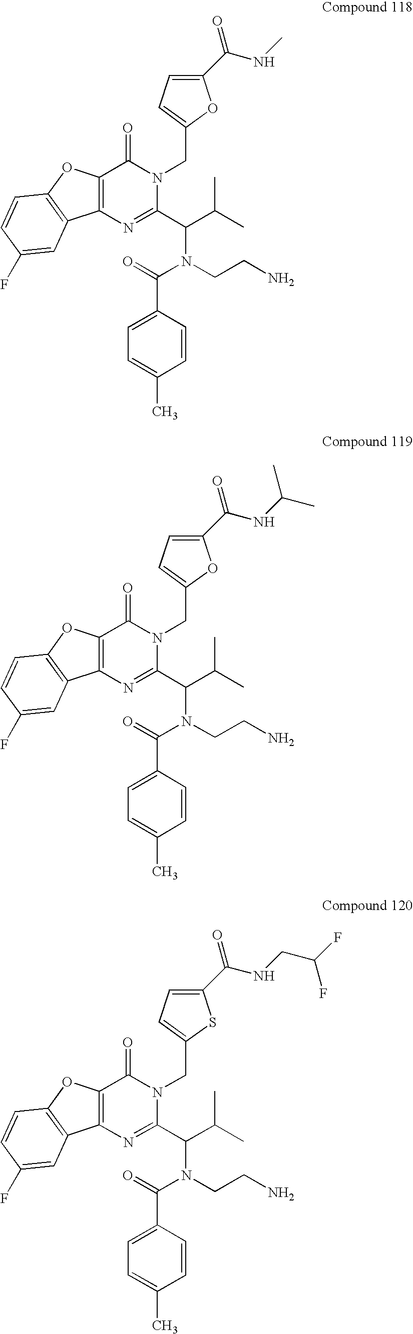

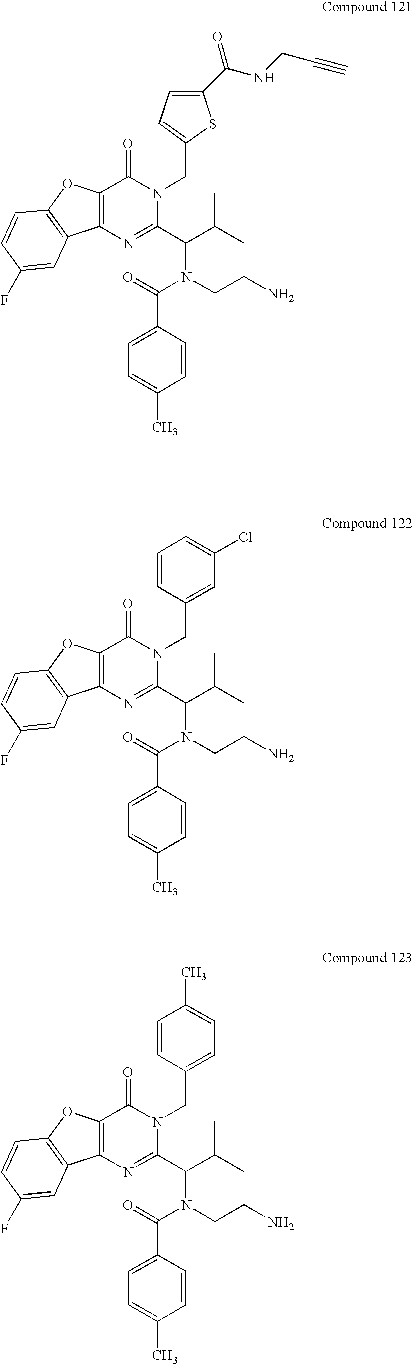

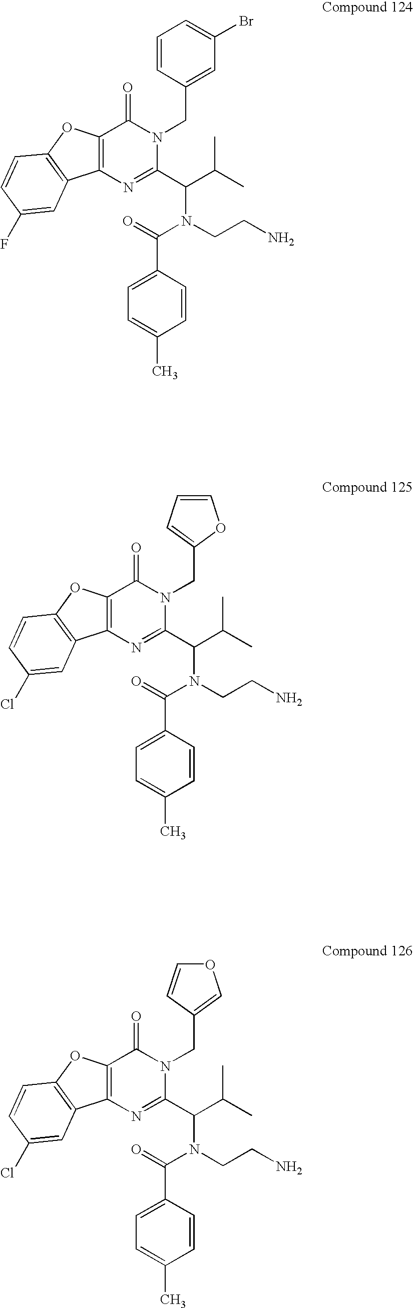

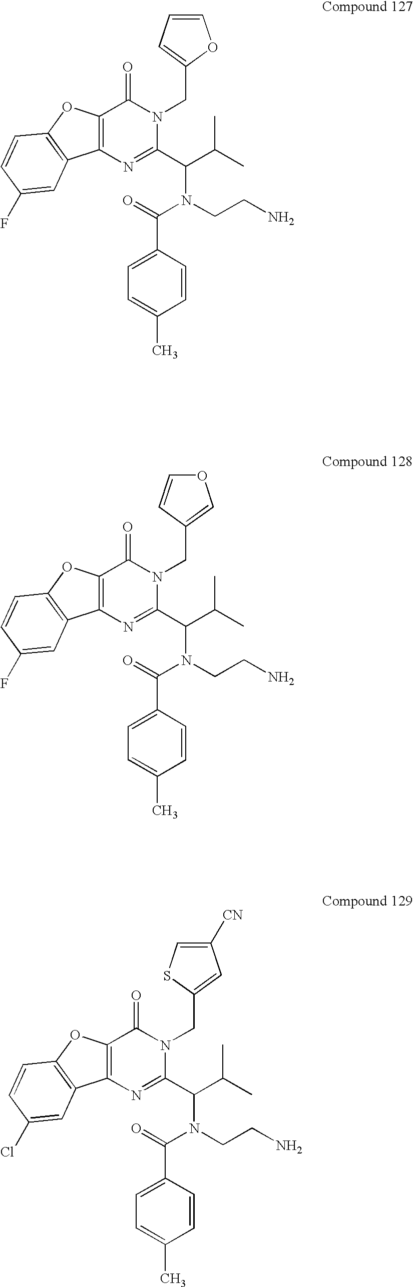

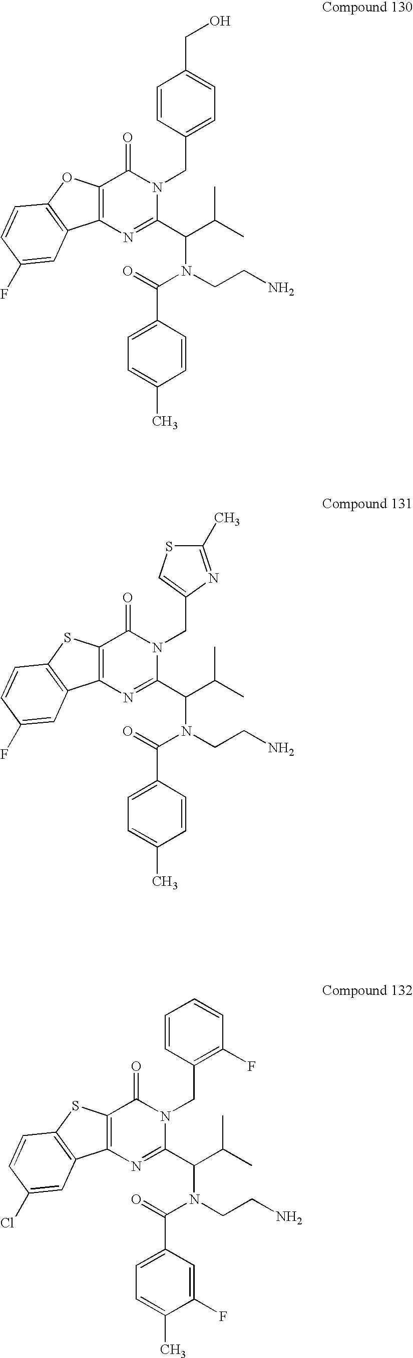

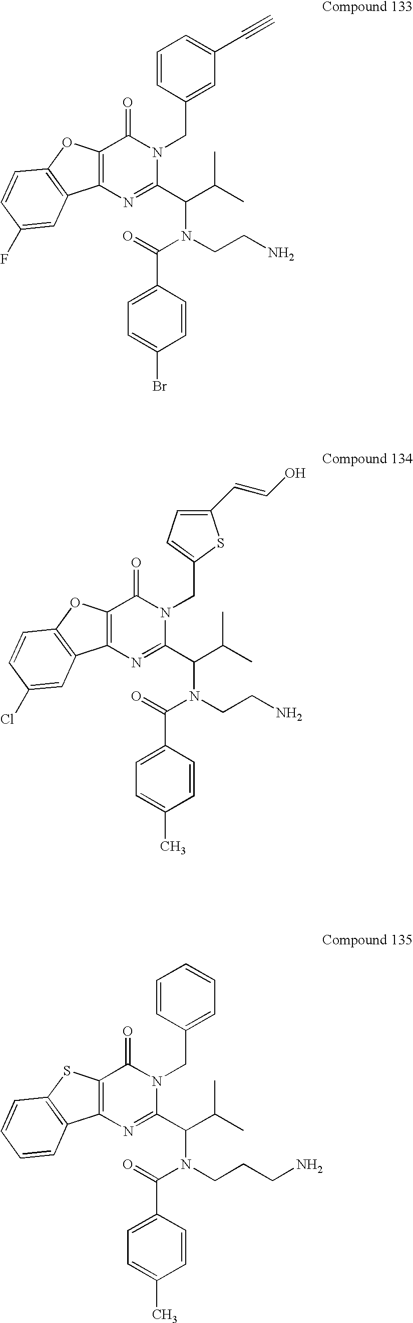

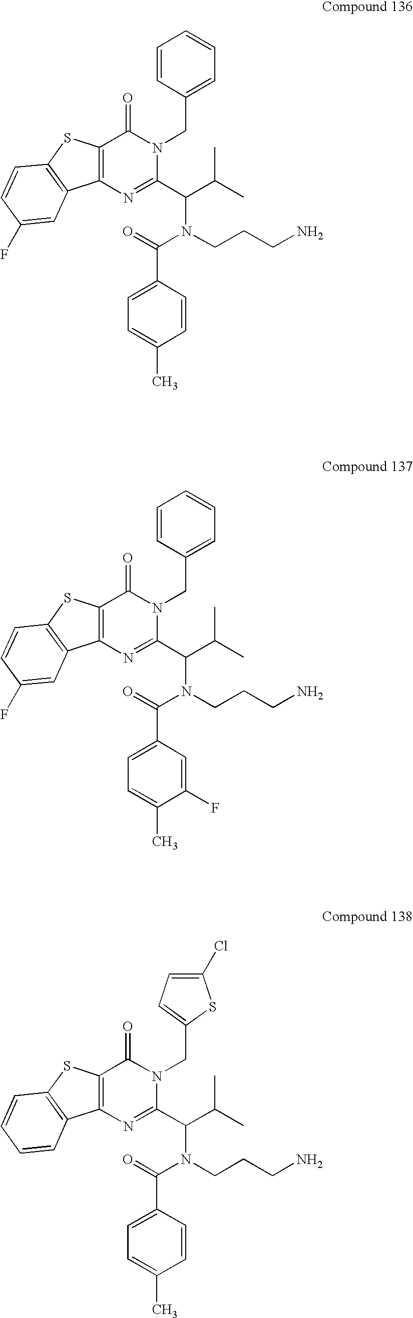

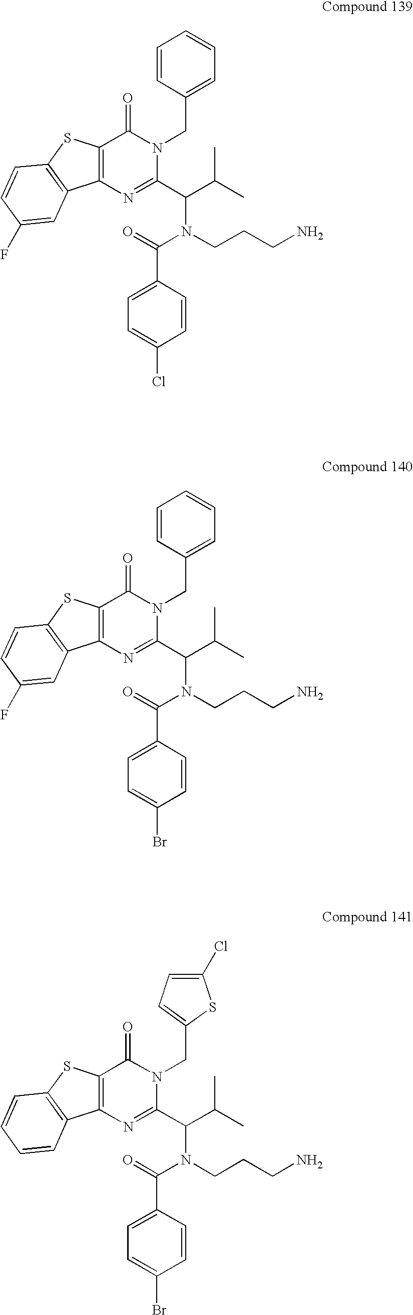

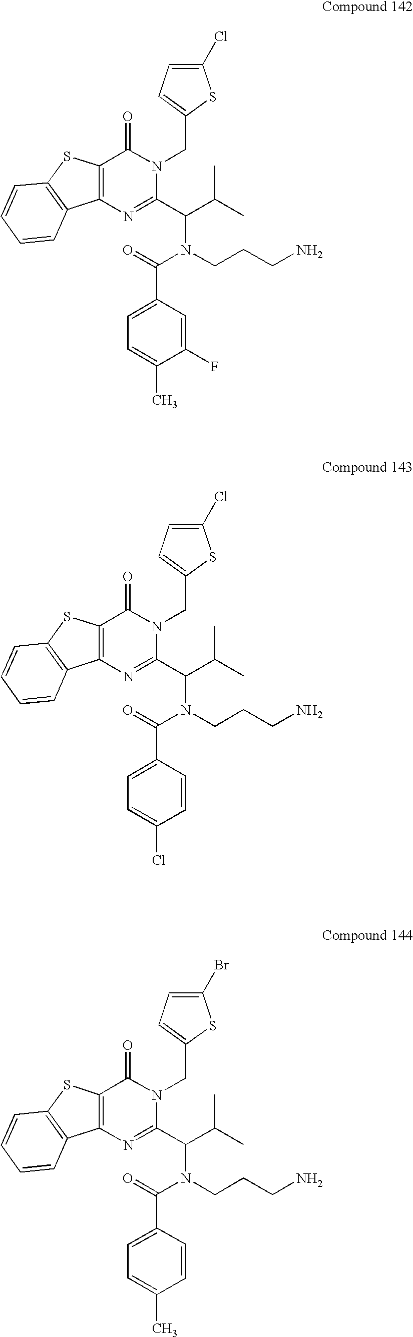

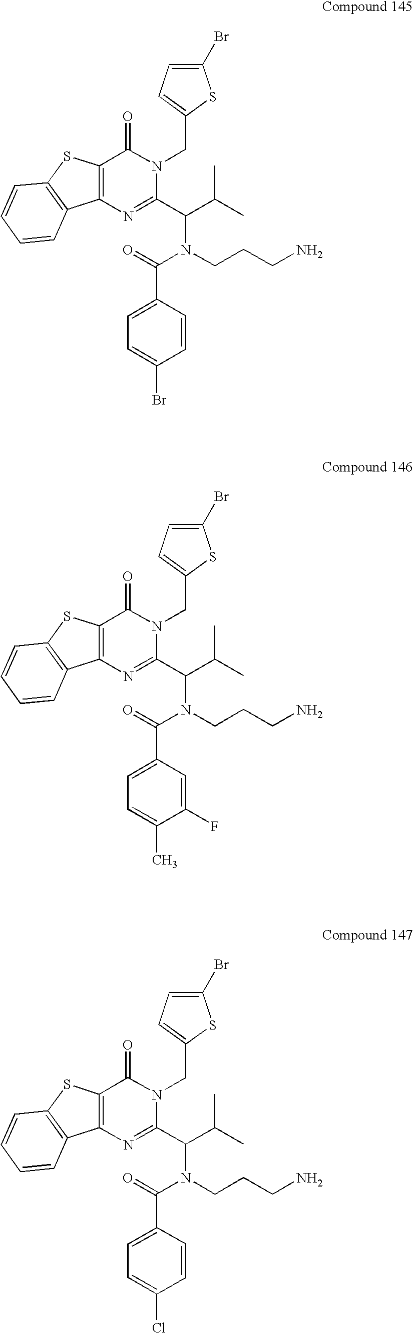

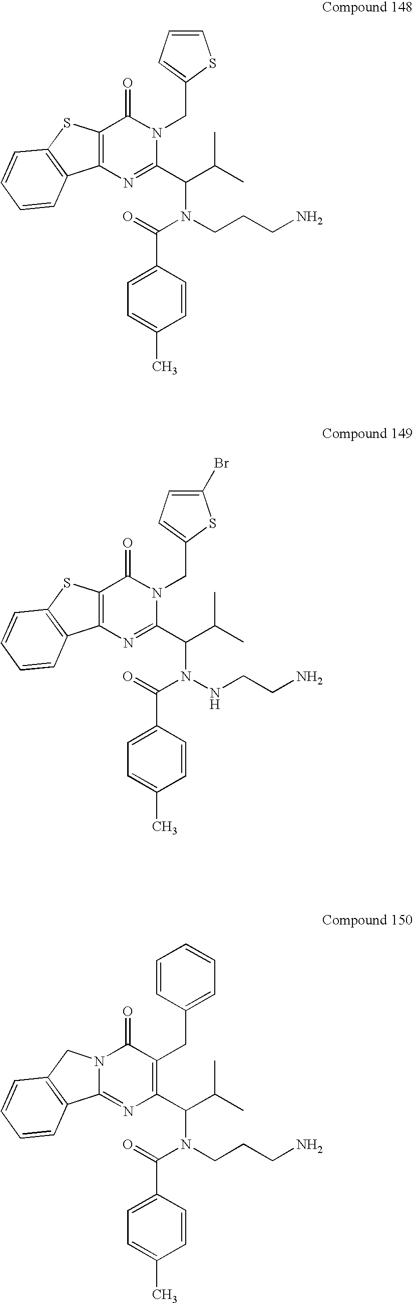

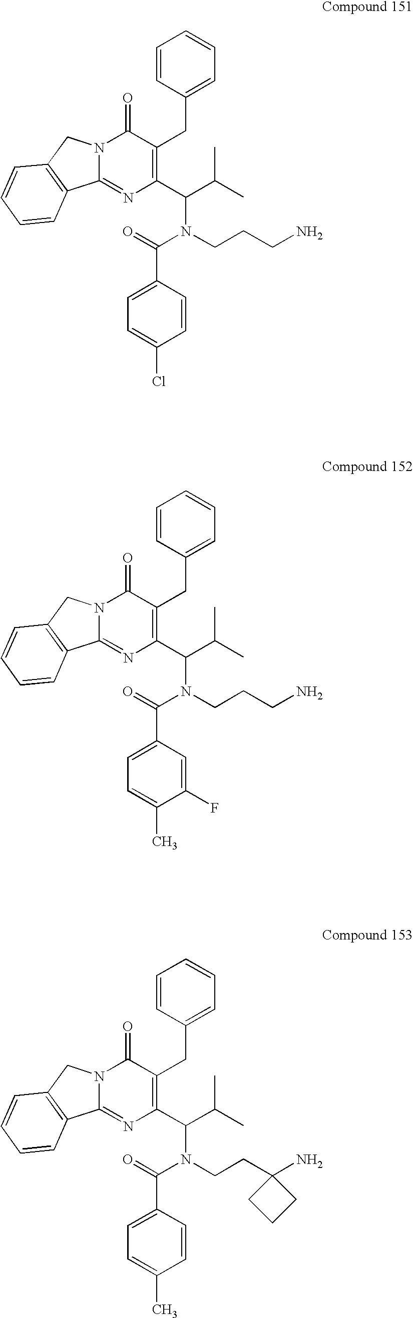

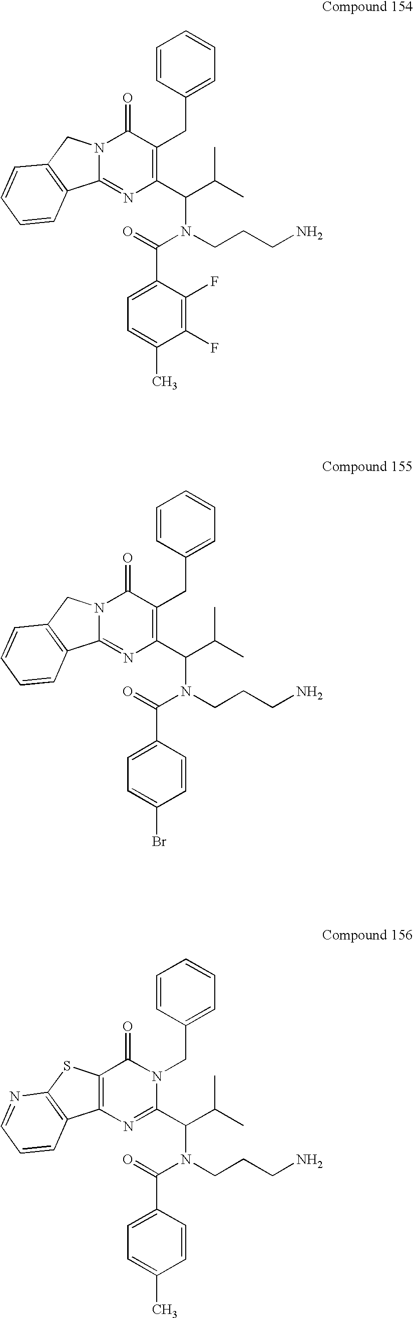

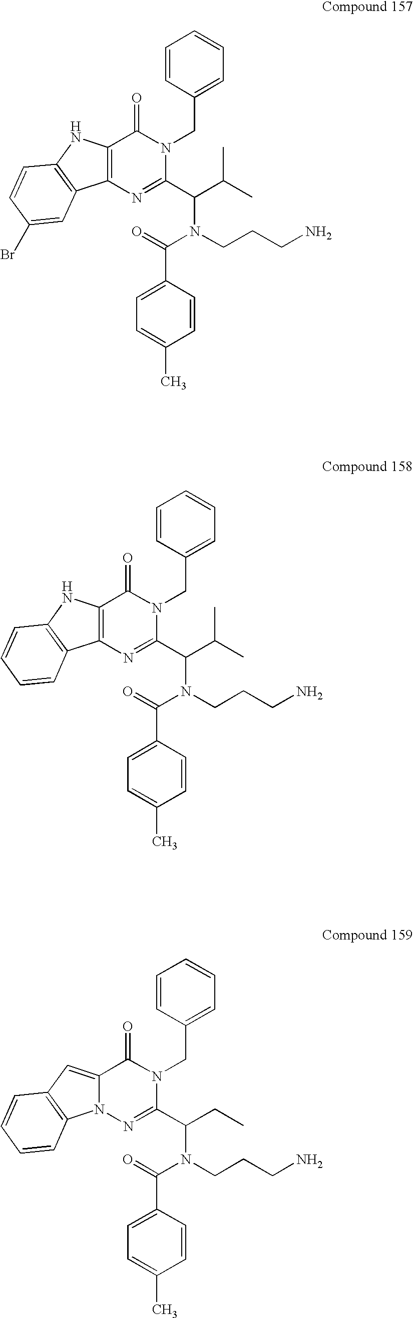

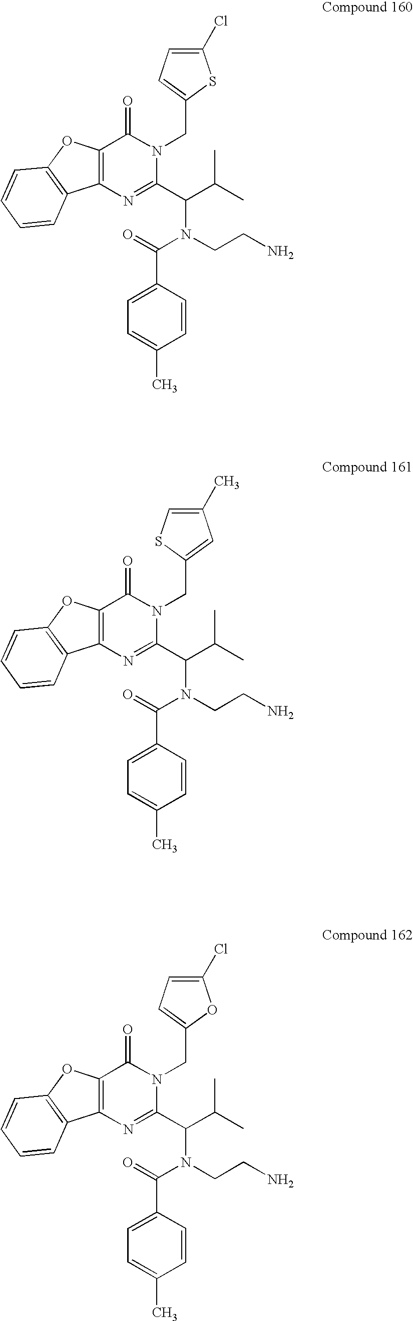

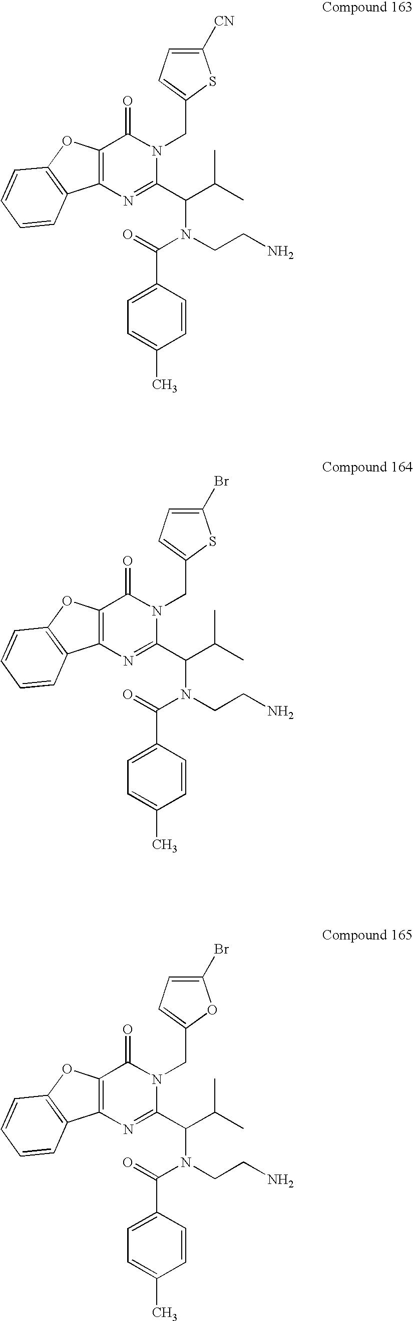

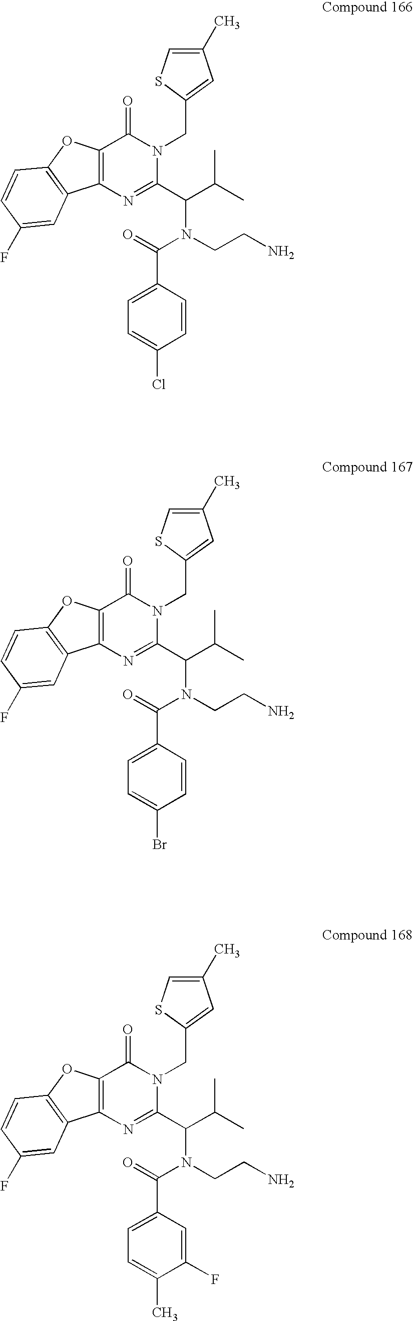

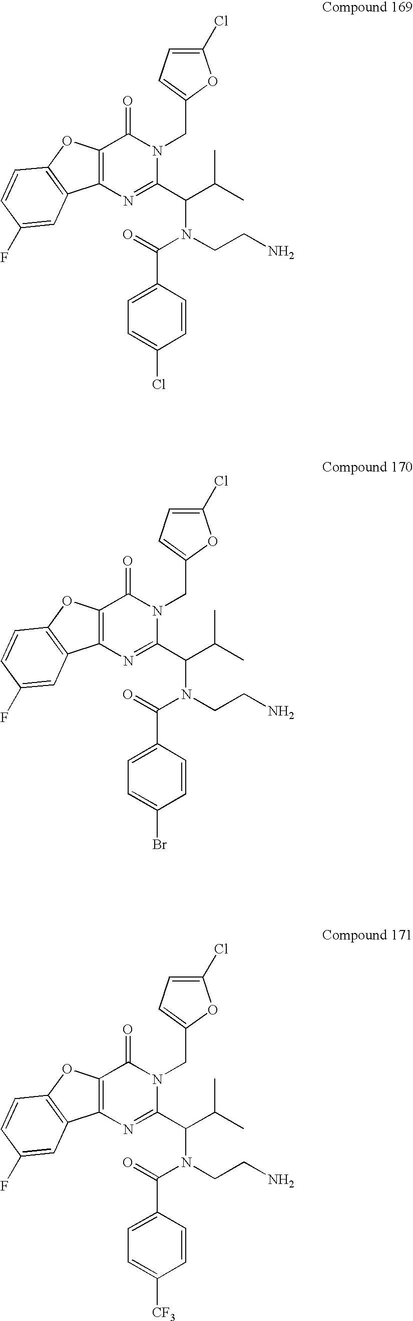

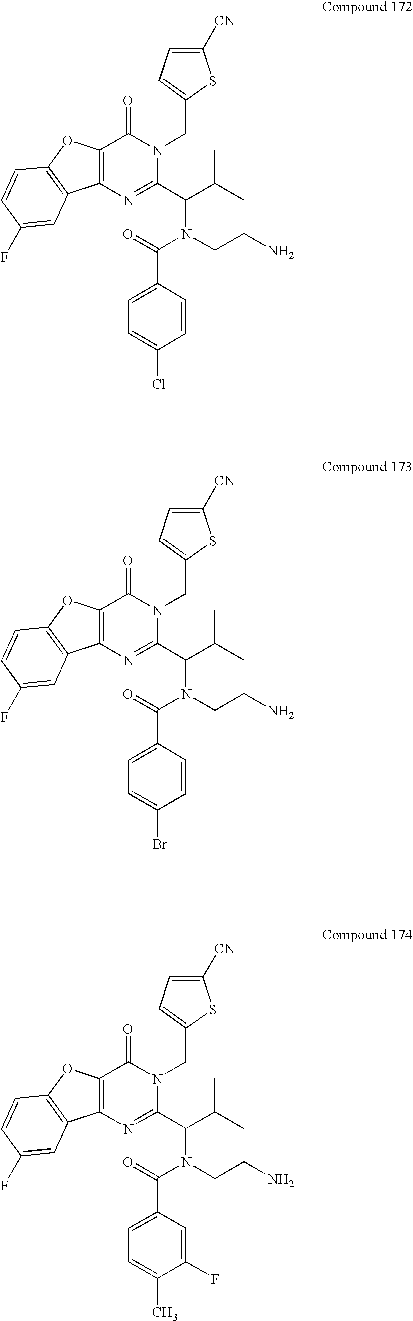

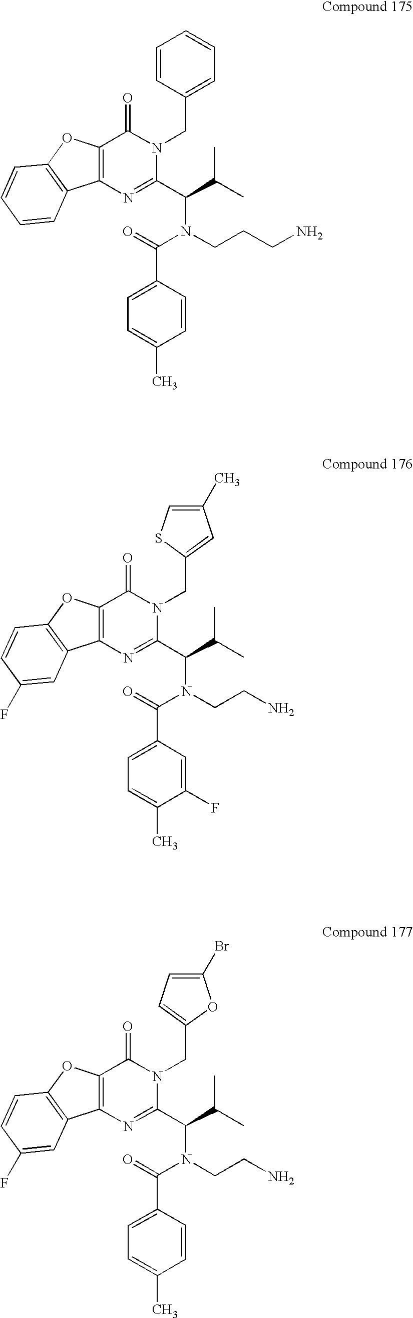

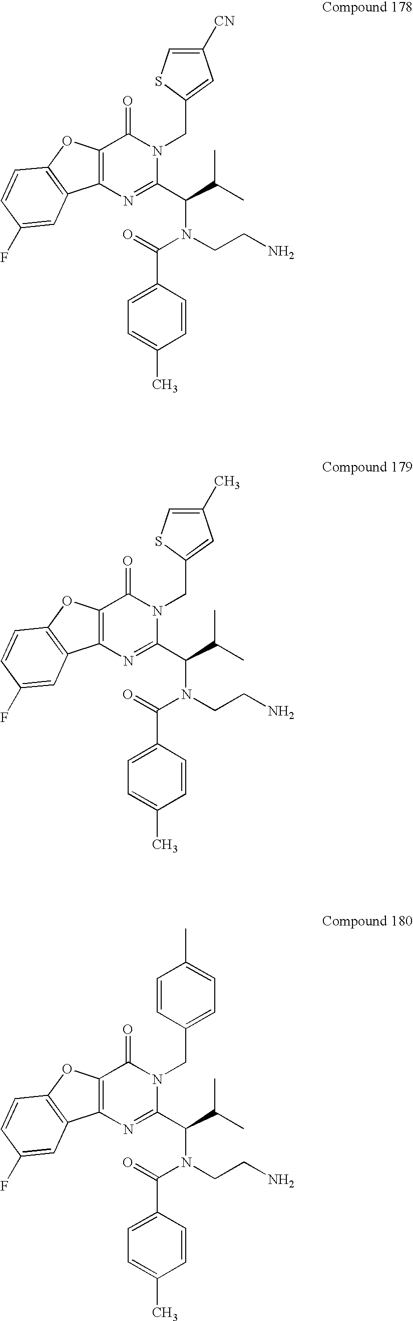

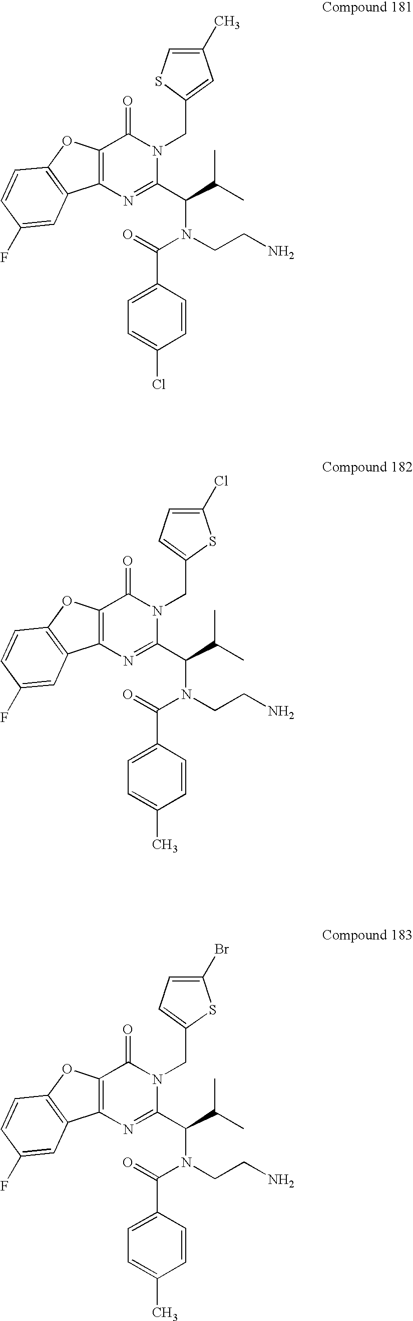

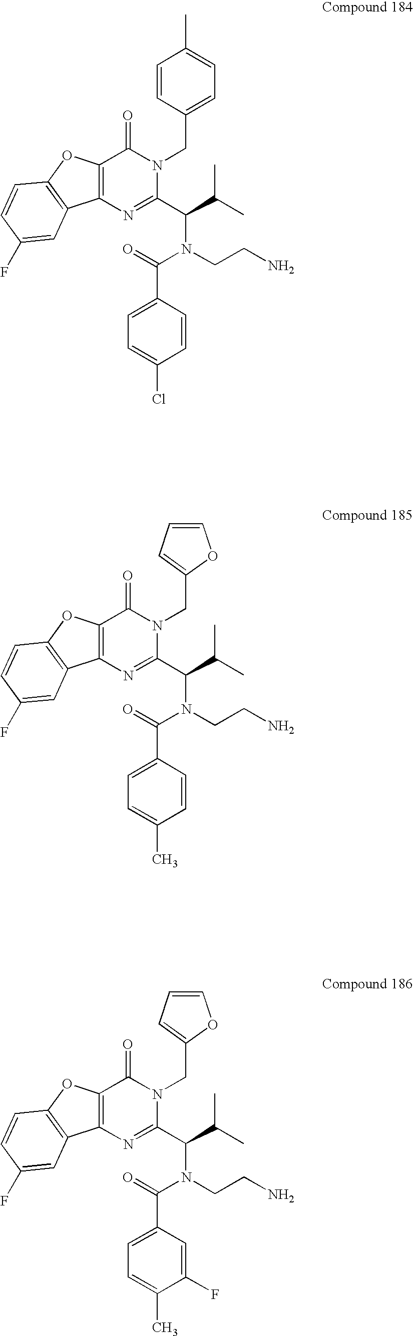

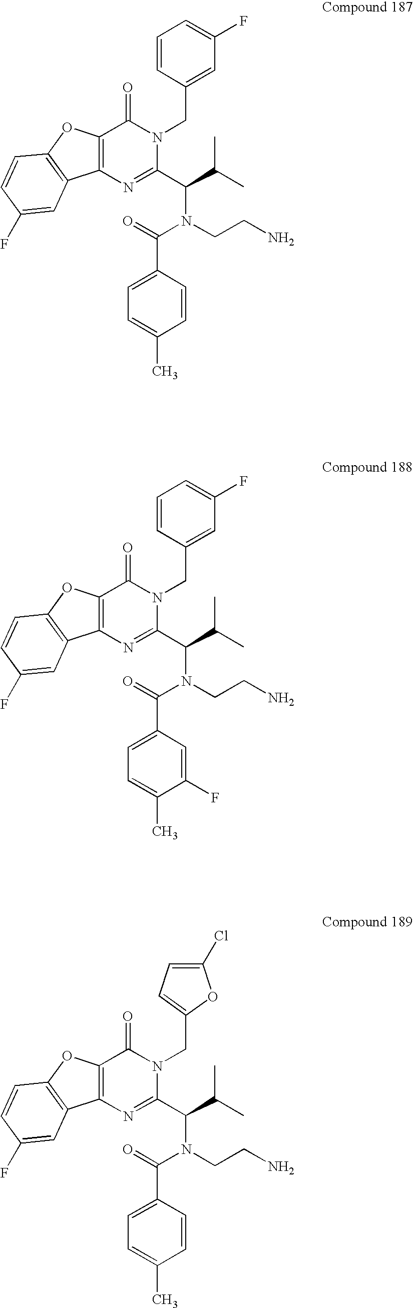

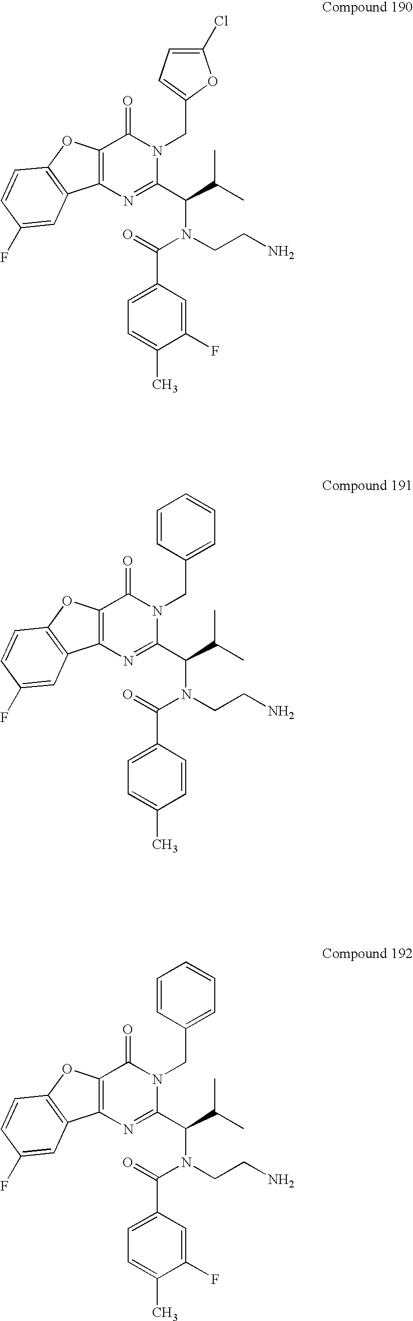

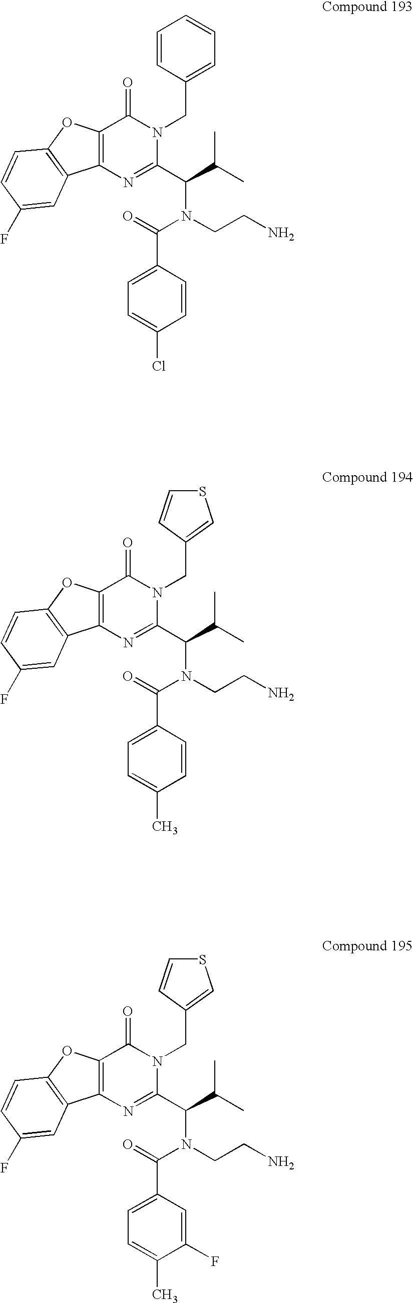

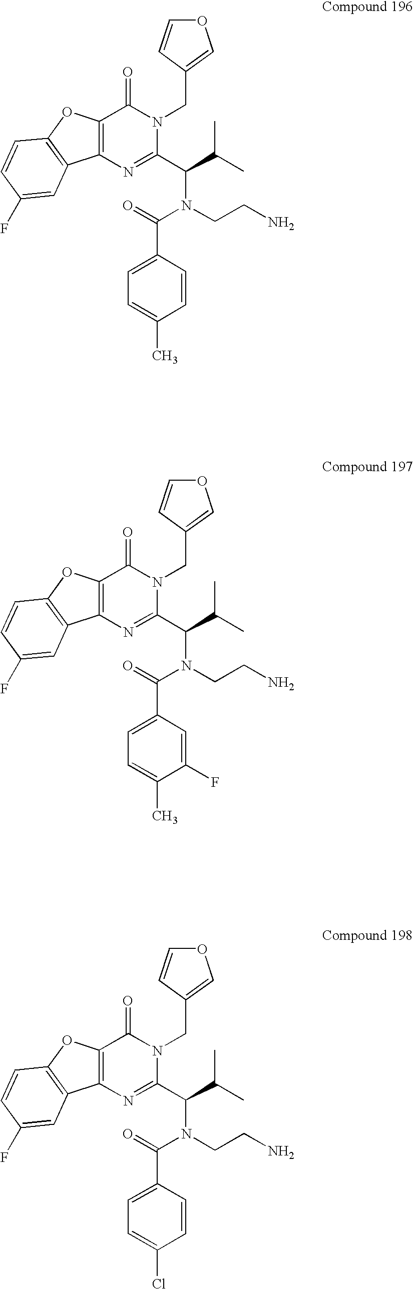

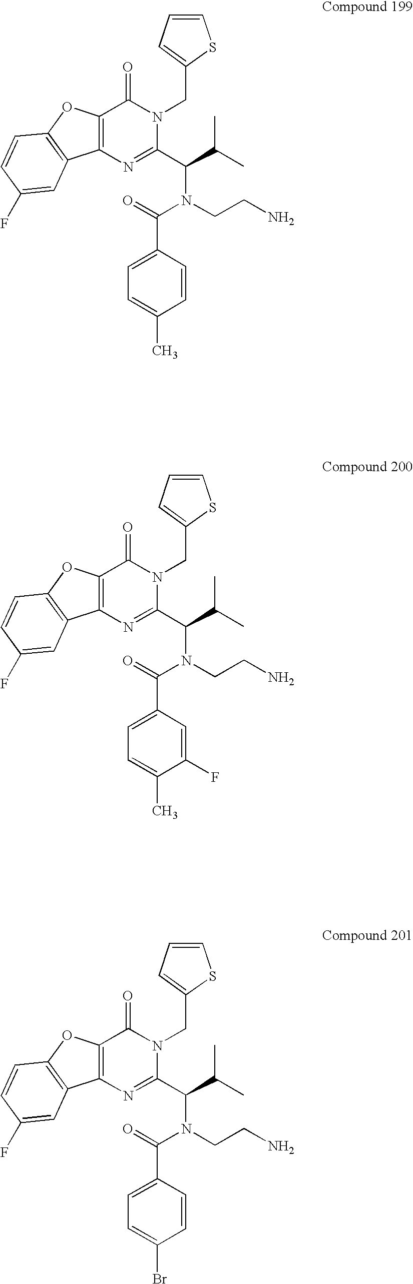

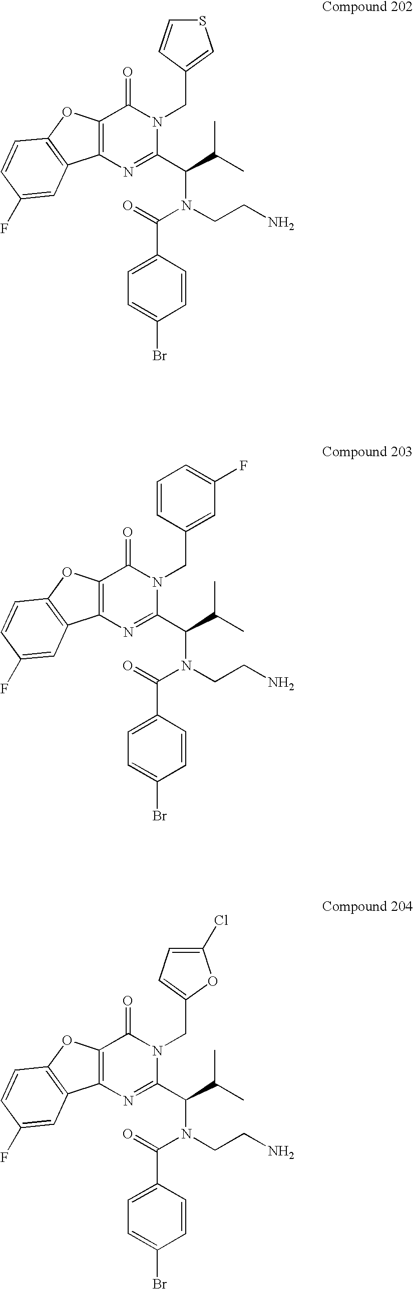

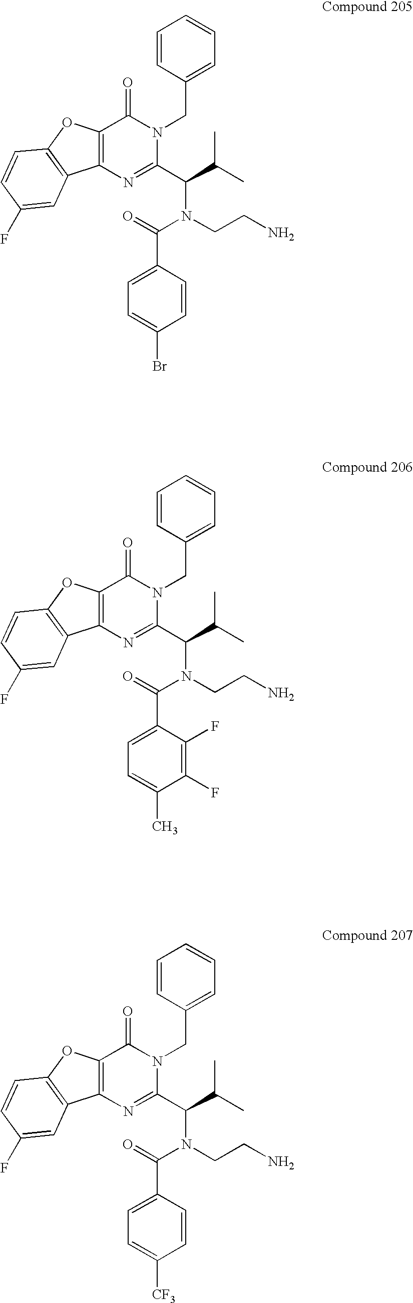

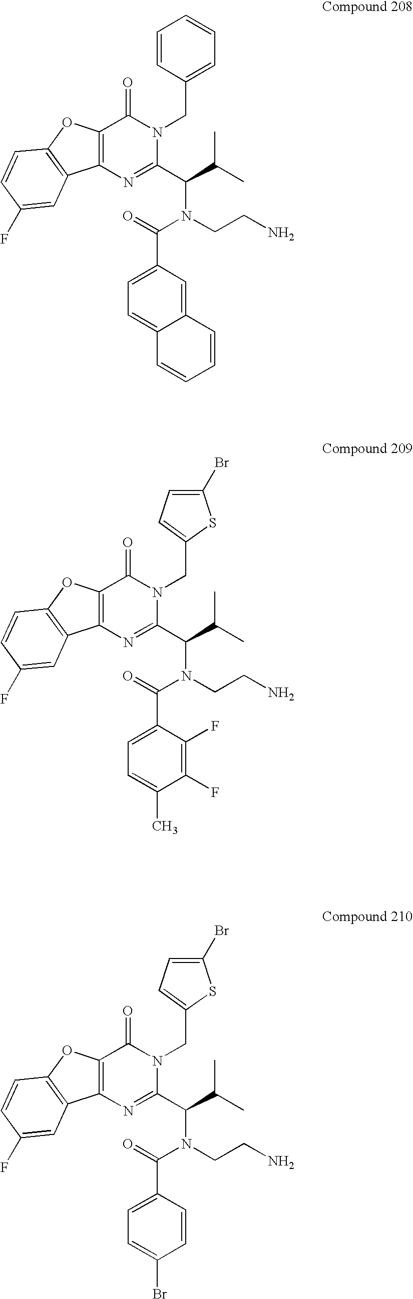

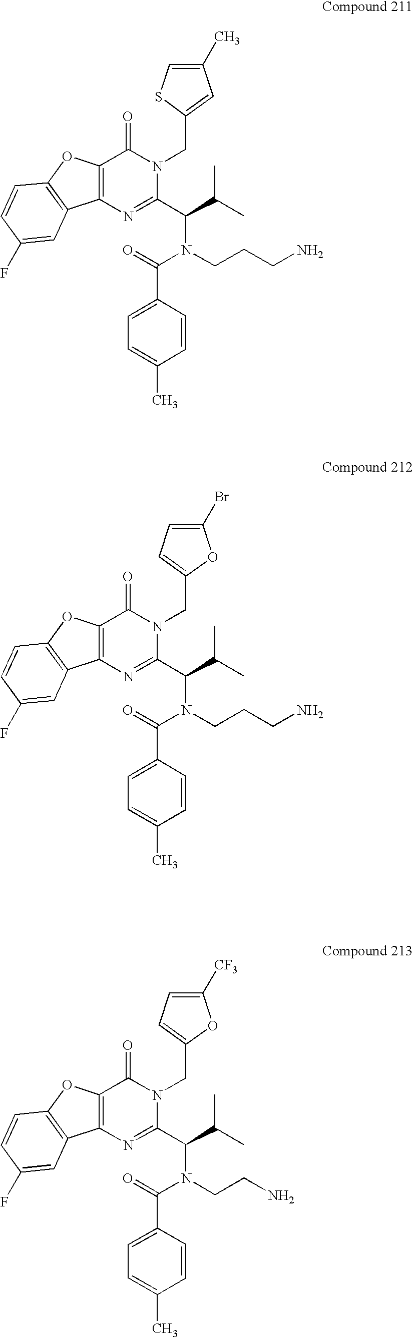

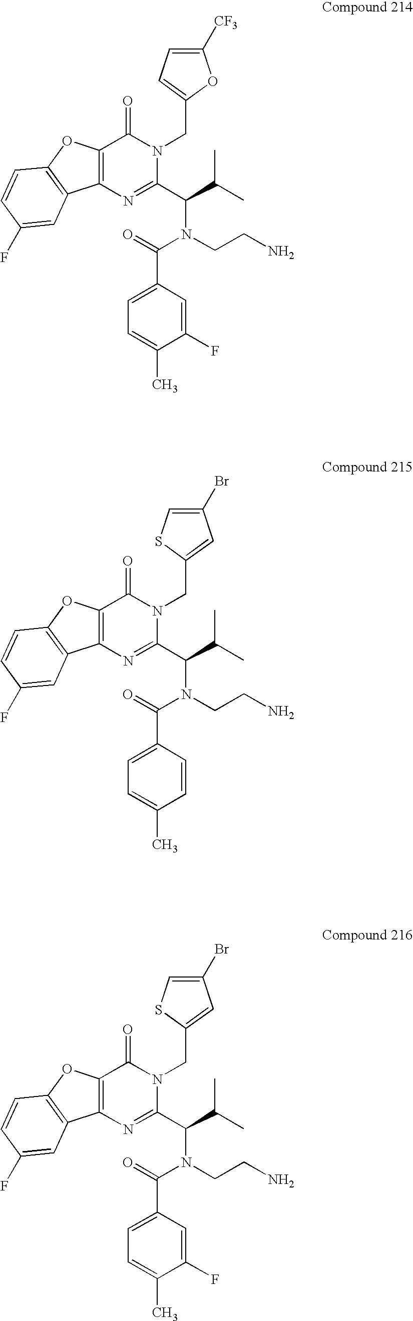

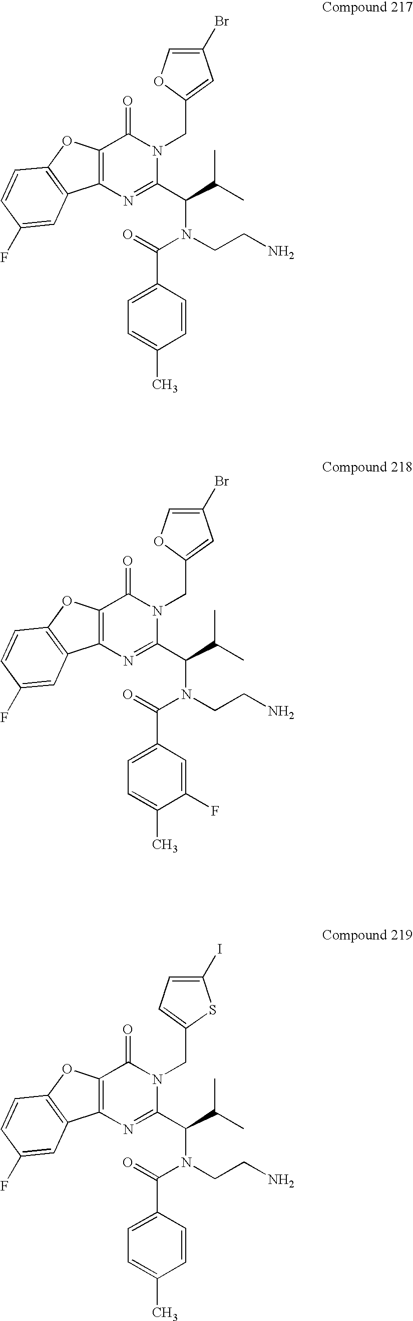

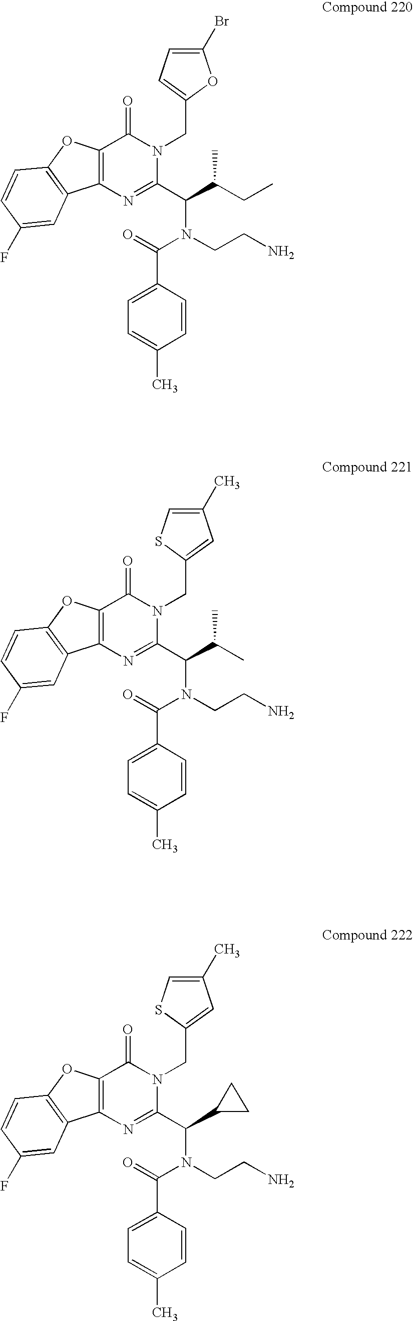

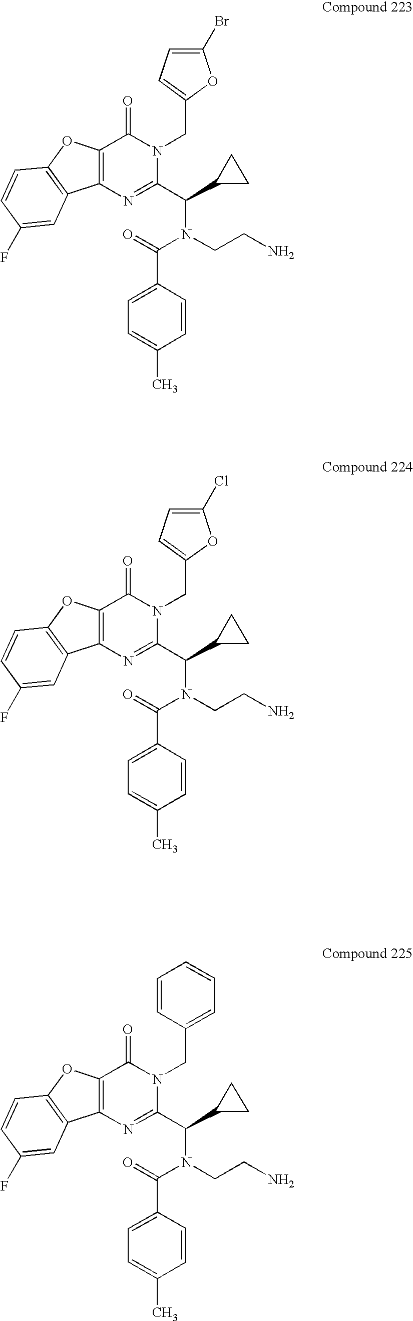

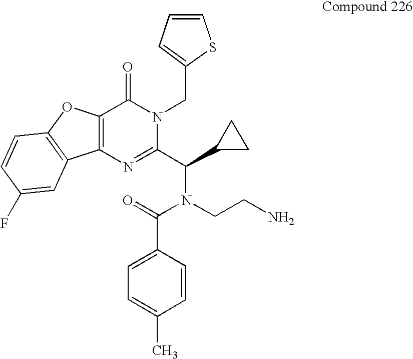

The compounds described above can be prepared by methods well known in the art, as well as the methods described herein. Examples 1-226 below provide detailed descriptions of the preparation of compounds 1-226 of this invention.

Scheme I shown below depicts a typical synthetic route for synthesizing certain exemplary compounds. In this scheme, A, P, Q, L₁, R₂, R₃, and R can be those groups defined in the Summary section above. Specifically, a bicyclic compound (e.g., compound A) containing an amino group and a carboxylate/amide group can first react with an acyl chloride to form an amide compound (e.g., compound B). When the amide compound contains a carboxylate group, this group can be protected (e.g., by forming an ester) before the amidation reaction and then deprotected after the amidation reaction. The amide compound can then undergo a ring closure reaction to form a tricyclic compound (e.g., compound C), which can then react with an amino compound or a halide compound to form a pyrimidinone compound (e.g., compound D). The pyrimidinone compound can subsequently react with bromine to form a brominated compound (e.g., compound E). The bromo group on the brominated compound can then be replaced with a secondary amino group to form an amino compound (e.g., compound F), which can react with substituted benzoyl chloride to form a compound of this invention. The bromo group can also be first replaced with an azide group (e.g., by reacting with sodium azide), which can be reduced to form a primary amino group (e.g., by reacting with triphenylphosphine). The primary amino group can then react with an aldehyde compound to form a compound having a secondary amino group (e.g., compound F) for use in the preparation of a compound of this invention.

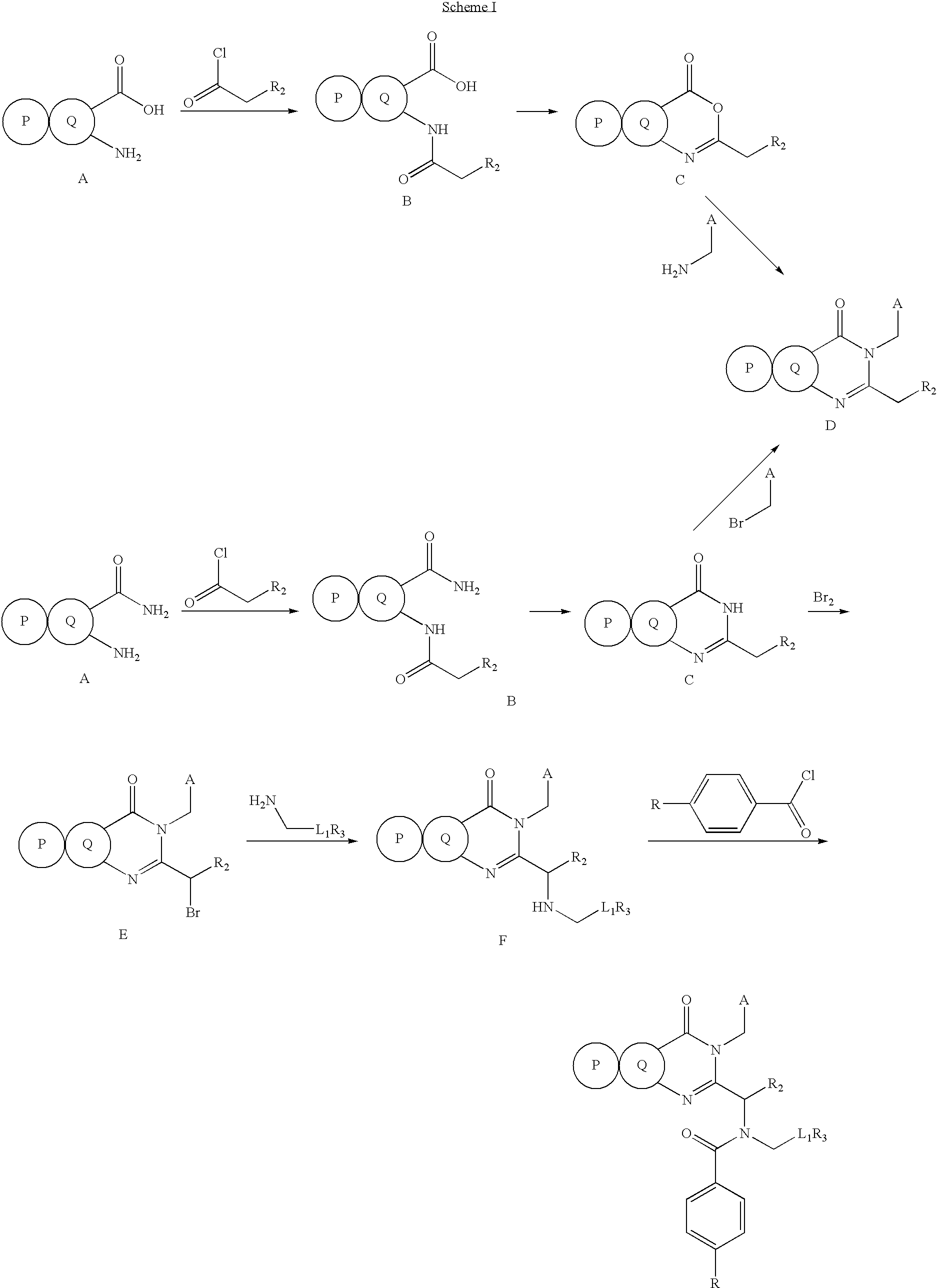

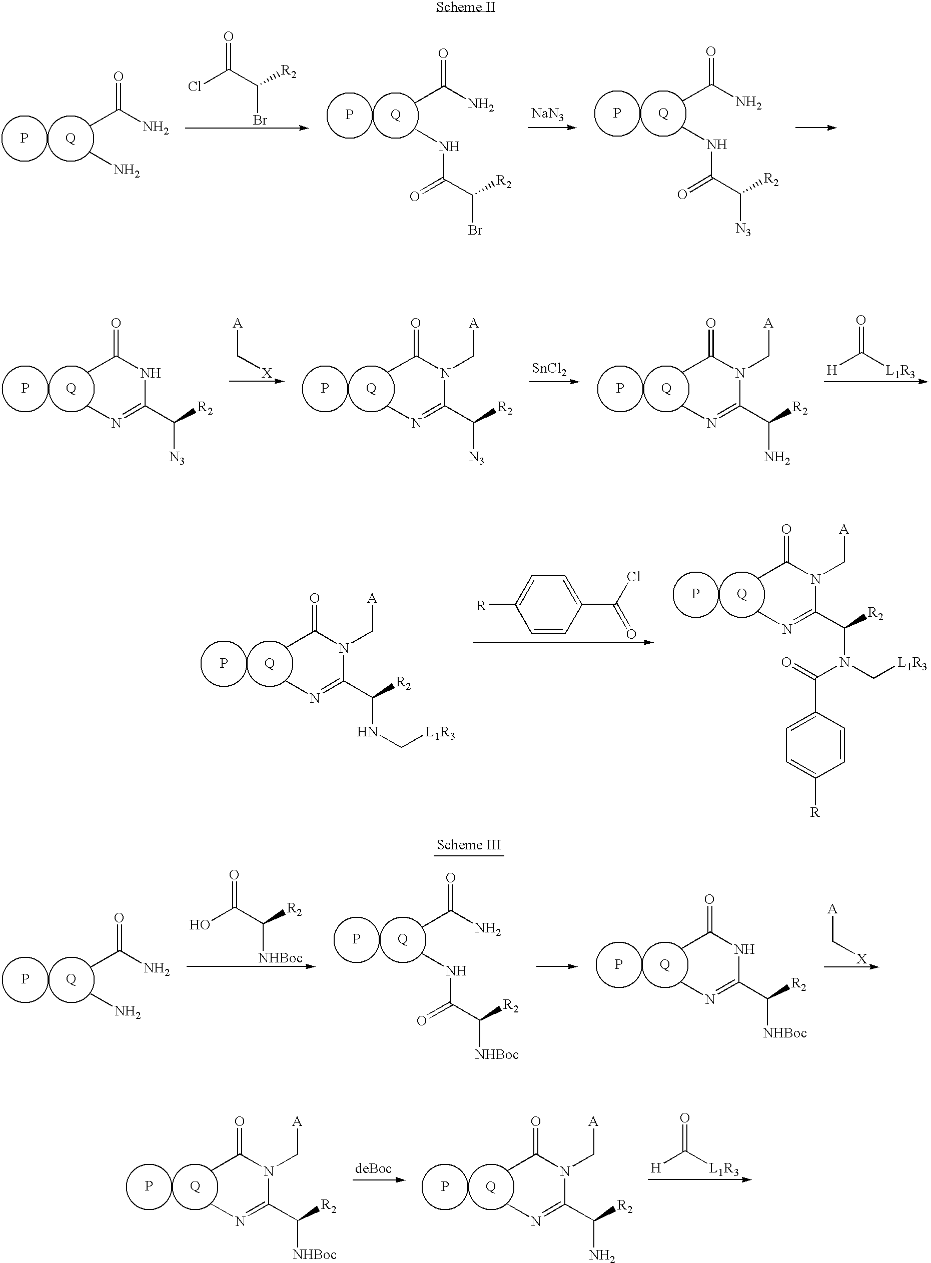

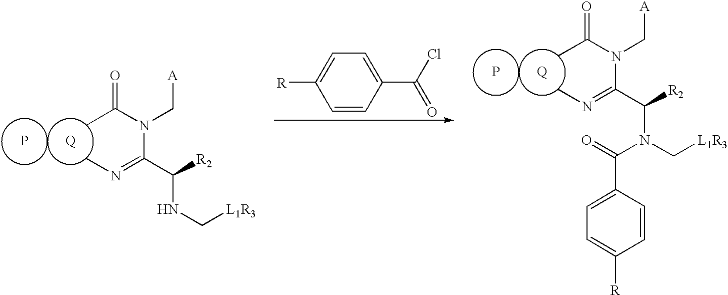

A compound thus synthesized can be purified by a method such as column chromatography, high-pressure liquid chromatography, or recrystallization.

Other above-mentioned compounds can be prepared using other suitable starting materials through the above synthetic routes and others known in the art. The methods described above may also additionally include steps, either before or after the steps described specifically herein, to add or remove suitable protecting groups in order to ultimately allow synthesis of the compounds described above. In addition, various synthetic steps may be performed in an alternate sequence or order to give the desired compounds. Synthetic chemistry transformations and protecting group methodologies (protection and deprotection) useful in synthesizing applicable compounds described above are known in the art and include, for example, those described in R. Larock, Comprehensive Organic Transformations, VCH Publishers (1989); T. W. Greene and P. G. M. Wuts, Protective Groups in Organic Synthesis, 2^{nd} Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Also within the scope of this invention is a pharmaceutical composition containing an effective amount of at least one compound of this invention and a pharmaceutical acceptable carrier. Further, this invention covers a method of administering an effective amount of one or more of the compounds of this invention to a patient having a disorder described in the summary section. “An effective amount” refers to the amount of an active compound of this invention that is required to confer a therapeutic effect on the treated subject. Effective doses will vary, as recognized by those skilled in the art, depending on the types of diseases treated, route of administration, excipient usage, and the possibility of combination with other therapeutic treatment.

To practice the method of the present invention, a composition having one or more compounds described above can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having one or more active compounds described above can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound described above. Examples of other carriers include colloidal silicon oxide, magnesium stearate, cellulose, sodium lauryl sulfate, and D&C Yellow # 10.

The compounds of this invention can be preliminarily screened for their efficacy in treating above-described disorders by in vitro and in vivo assays (See Examples 227-230 below) and then confirmed by clinic trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Preparation of Compound 1

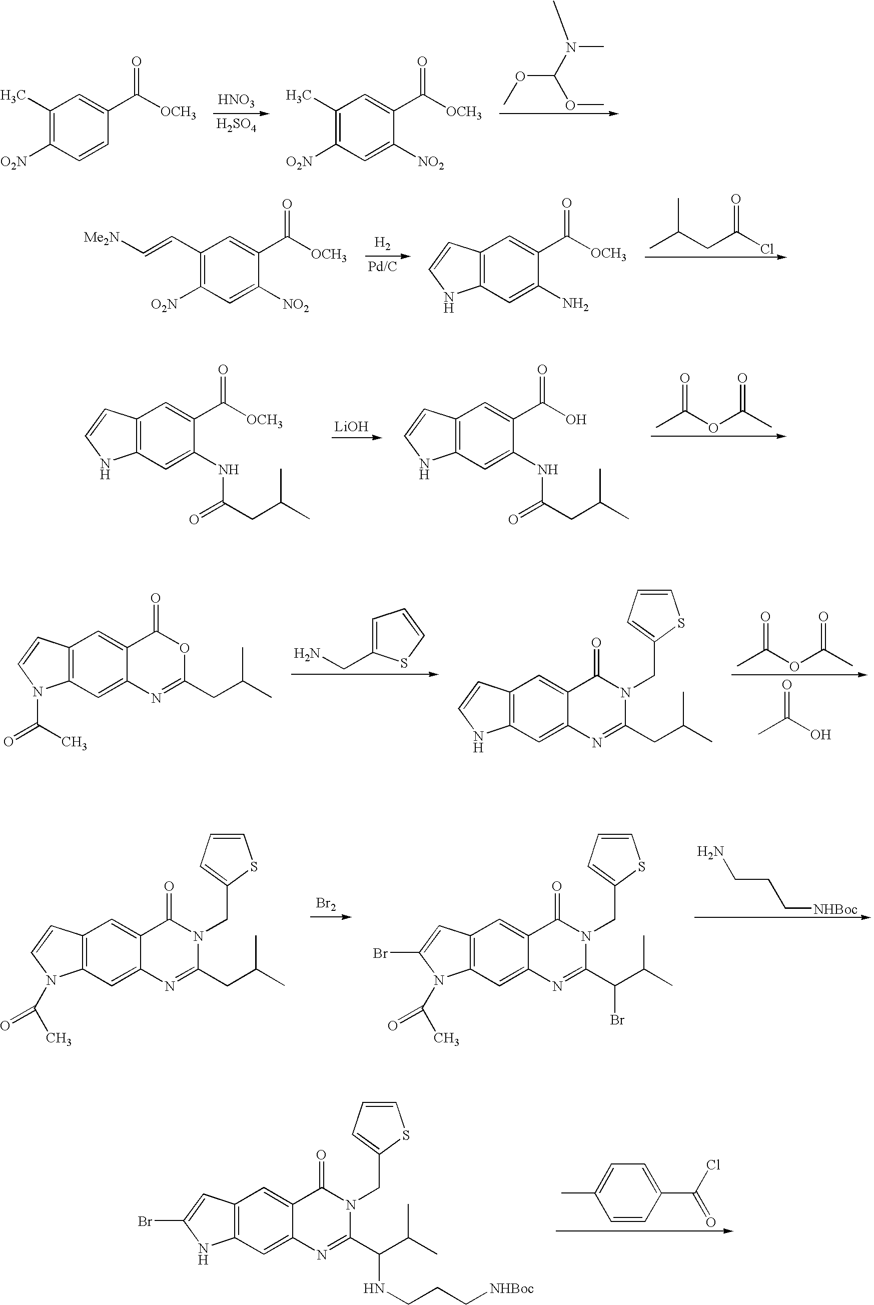

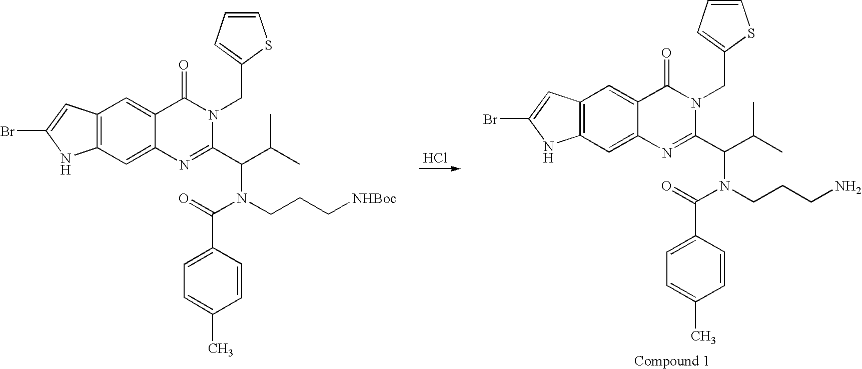

A solution of 3-methyl-4-nitro-benzoic acid methyl ester (10.12 g, 51.85 mmol) in 44 mL of concentrated H₂SO₄ was cooled to −10° C. 10.3 mL of fuming HNO₃ was added dropwise over 45 minutes to maintain the reaction temperature at −10° C. The solution was maintained at −20° C. till the reaction was complete. The mixture was poured into 600 mL of crushed ice and the suspension was extracted with ethyl acetate (3×400 mL). The combined extract was washed with a 5% NaOH aqueous solution until the aqueous phase remained neutral. The extract was then dried and concentrated to give 12.3 g of oil. The oil was dissolved in 200 mL of hot i-PrOH and the solution was allowed to slowly cool down to 25° C. The precipitate thus formed was collected by filtration, washed with i-PrOH, and dried under reduced pressure to give 9.33 g of the crude 5-methyl-2,4-dinitro-benzoic acid methyl ester, which was used in the next step without further purification. LC-MS (M+H): 262.9.

A solution of the crude 5-methyl-2,4-dinitro-benzoic acid methyl ester (9.33 g, 39.87 mmol) in 125 mL of toluene was added of dimethoxy-N,N-dimethylmethanamine (13.88 mL, 116.6 mmol). The mixture was stirred at refluxing temperature for 4 days. The reaction mixture was concentrated under vacuum and dried under reduced pressure to give 6.06 g of the crude 5-(2-dimethylamino-vinyl)-2,4-dinitro-benzoic acid methyl ester. LC-MS (M+H): 295.9.

10% Pd/C (1.09 g, 0.08 mmol) was added to a solution of the crude 5-(2-dimethylamino-vinyl)-2,4-dinitro-benzoic acid methyl ester (4.00 g, 13.6 mmol) in 180 mL of EtOH. The mixture was subject to hydrogenation at 60 psi till the reaction was complete. After the ethanol solution was allowed to pass through a Celite bed to remove Pd/C, it was concentrated under reduced pressure. The resultant crude product was purified by silica gel column chromatography using 20% ethyl acetate/hexane as an eluant to give 2.50 g of 6-amino-1H-indole-5-carboxylic acid methyl ester. LC-MS (M+H): 191.1.

Isovaleryl chloride (0.92 mL, 7.5 mmol) was added dropwise to a stirred solution of 6-amino-1H-indole-5-carboxylic acid methyl ester (1.30 g, 6.8 mmol) in 14 mL of dry DMF at room temperature. The reaction mixture was stirred at room temperature for 25 hours. The mixture was then poured into 200 mL of water and stirred for 1 hour. The precipitate thus formed was collected by filtration and dried under reduced pressure to give 1.71 g of the 6-(3-methyl-butyrylamino)-1H-indole-5-carboxylic acid methyl ester. LC-MS (M+H): 275.1.

12.5 mL of an 1 N LiOH aqueous solution was added to a mixture of 6-(3-methyl-butyrylamino)-1H-indole-5-carboxylic acid methyl ester (1.71 g, 6.24 mmol) in 32 mL of THF. The mixture was stirred at 80° C. for 16 hours and then concentrated in vacuo. The residue was dissolved in water and treated with an 1N HCl aqueous solution till its pH reached 3-4. The precipitate thus formed was collected by filtration and dried under reduced pressure to give 1.40 g of the 6-(3-methyl-butyrylamino)-1H-indole-5-carboxylic acid. LC-MS (M+H): 261.1.

A stirred solution of 6-(3-methyl-butyrylamino)-1H-indole-5-carboxylic acid (1.40 g, 5.44 mmol) in 50 mL of acetic anhydride was heated at refluxing temperature for 30 hours. Excess acetic anhydride and by-product acetic acid were removed by distillation. The resultant residue was concentrated under vacuum and dried under reduced pressure to give 1.77 g of 1-acetyl-7-isobutyl-1H-6-oxa-1,8-diaza-cyclopenta[b]naphthalene-5-one. LC-MS (M+H): 285.0.

Thiophen-2-ylmethanamine (0.7 mL, 3.05 mmol) was added to a solution of 1-acetyl-7-isobutyl-1H-6-oxa-1,8-diaza-cyclopenta[b]naphthalene-5-one (1.77 g, 6.20 mmol) in 31 mL of toluene. The mixture was stirred at refluxing temperature for 3.7 days. The mixture was concentrated under vacuum and dried under reduced pressure. The resultant crude product was suspended in 30 mL of ethylene glycol, followed by addition of sodium hydroxide (0.45 g, 11.25 mmol). The mixture was stirred at 140° C. for 6 hours. The mixture was then concentrated and treated with a 2N HCl aqueous solution till pH reached 8-9. The mixture was concentrated in vacuo and subsequently re-dissolved in ethyl acetate. The ethyl acetate layer was washed with water and brine, dried over anhydrous MgSO₄, and concentrated in vacuo to give 2.32 g of 2-isobutyl-3-(thiophen-2-ylmethyl)-3H-pyrrolo[3,2-g]quinazolin-4(8H)-one. LC-MS (M+H): 338.1.

1 mL of acetic acid was added to a stirred solution of 2-isobutyl-3-(thiophen-2-ylmethyl)-3H-pyrrolo[3,2-g]quinazolin-4(8H)-one (2.32 g, 6.88 mmol) in 34 mL of acetic anhydride. The mixture was stirred at refluxing temperature for 24 hours. Acetic acid and acetic anhydride were then removed by distillation. The resultant mixture was concentrated under vacuum and dried under reduced pressure. The crude product was then purified by silica gel column chromatography using 20% ethyl acetate/hexane as an eluent to give 1.03 g of pure 1-acetyl-7-isobutyl-6-thiophen-2-ylmethyl-1,6-dihydro-1,6,8-triaza-cyclopenta[b]naphthalen-5-one. LC-MS (M+H): 380.0.

To a stirred solution of 1-acetyl-7-isobutyl-6-thiophen-2-ylmethyl-1,6-dihydro-1,6,8-triaza-cyclopenta[b]naphthalen-5-one (0.178 g, 0.47 mmol) and sodium acetate (0.19 g, 2.35 mmol) in 3.5 mL of glacial acetic acid was added a solution of bromine (0.025 mL, 0.47 mmol) in 1.2 mL of glacial acetic acid dropwise over 10 minutes via an addition funnel. The reaction mixture was stirred at room temperature for 5 hours and concentrated in vacuo. The crude material was dissolved in dichloromethane and washed with water and brine, dried over anhydrous MgSO₄, and concentrated in vacuo. The crude product was purified by silica gel column chromatography using 15% ethyl acetate/hexane as an eluant to give 0.10 g of 1-acetyl-2-bromo-7-(1-bromo-2-methyl-propyl)-6-thiophen-2-ylmethyl-1,6-dihydro-1,6,8-triaza-cyclopenta[b]naphthalen-5-one product. ¹H NMR (CDCl₃) δ 8.65 (s, 1H), 8.45 (s, 1H), 7.58 (s, 1H), 7.16-7.19 (m, 1H), 6.98 (d, J=3.3, 1H), 6.87-6.90 (m, 1H), 6.12 (ABq, J=15.8, 1H), 5.08 (ABq, J=15.8, 1H), 4.04 (d, J=9.9, 1H), 2.81-2.88 (m, 1H), 2.61 (s, 3H), 1.19 (d, J=6.6, 3H), 0.71 (d, J 6.6, 3H). LC-MS (M+H): 538.0.

A mixture of 1-acetyl-2-bromo-7-(1-bromo-2-methyl-propyl)-6-thiophen-2-ylmethyl-1,6-dihydro-1,6,8-triaza-cyclopenta[b]naphthalen-5-one (0.08 g, 0.15 mmol) and tert-butyl 3-aminopropylcarbamate (0.13 g, 0.76 mmol) was stirred at 90° C. for 2 hours. The reaction mixture was stirred at room temperature for 5 hours and dissolved in dichloromethane. It was then washed with a saturated NaHCO₃ aqueous solution and brine, dried over anhydrous MgSO₄, and concentrated in vacuo. The crude product was purified by silica gel column chromatography using 50% ethyl acetate/hexane as an eluant to give 0.04 g of {3-[1-(2-bromo-5-oxo-6-thiophen-2-ylmethyl-5,6-dihydro-1H-1,6,8-triaza-cyclopenta[b]naphthalen-7-yl)-2-methyl-propylamino]-propyl}-carbamic acid tert-butyl ester. LC-MS (M+H): 590.2.

4-Methylbenzoyl chloride (0.01 mL, 0.08 mmol) was added to a stirred solution of {3-[1-(2-bromo-5-oxo-6-thiophen-2-ylmethyl-5,6-dihydro-1H-1,6,8-triaza-cyclopenta[b]naphthalen-7-yl)-2-methyl-propylamino]-propyl}-carbamic acid tert-butyl ester (0.04 g, 0.08 mmol) and triethylamine (0.02 mL, 0.15 mmol) in 0.75 mL of dried dichloromethane at 0° C. The mixture was stirred at room temperature for 23 hours, concentrated in vacuo, and re-dissolved in dichloromethane. The dichloromethane layer was washed with water and brine, dried over anhydrous MgSO₄, and concentrated in vacuo. The crude product was purified by silica gel chromatography with 15% ethyl acetate/hexane to give 0.01 g of {3-[[1-(2-bromo-5-oxo-6-thiophen-2-ylmethyl-5,6-dihydro-1H-1,6,8-triaza-cyclopenta[b]naphthalen-7-yl)-2-methyl-propyl]-(4-methyl-benzoyl)-amino]-propyl}-carbamic acid tert-butyl ester. ¹H NMR (CDCl₃) δ 8.62 (s, 1H), 7.92 (d, J=7.8, 1H), 7.28-7.44 (m, 3H), 7.20-7.38 (m, 3H), 6.83-6.95 (m, 2H), 6.03 (ABq, J=15.3, 1H), 5.90 (d, J=10.8, 1H), 5.46 (ABq, J=15.3, 1H), 4.05 (d, J=6.9, H), 3.48-3.59 (m, 1H), 3.32-3.48 (m, 1H), 2.72-2.89 (m, 2H), 2.31 (s, 3H), 1.38 (s, 9H), 1.00 (d, J=5.4, 3H), 0.73-0.88 (m, 2H), 0.53 (d, J=5.4, 3H). LC-MS (M+H): 706.2.

0.10 mL of 4N HCl in dioxane solution was added dropwise at 0° C. to a stirred solution of {3-[[1-(2-bromo-5-oxo-6-thiophen-2-ylmethyl-5,6-dihydro-1H-1,6,8-triaza-cyclopenta[b]naphthalen-7-yl)-2-methyl-propyl]-(4-methyl-benzoyl)-amino]-propyl}-carbamic acid tert-butyl ester (0.01 g, 0.02 mmol) in 0.10 mL of dichloromethane. The mixture was slowly warmed up to room temperature and stirred for 3 hours. It was then concentrated in vacuo, washed with ether, and dried under high vacuum to give 0.008 g of Compound 1. LC-MS (M+H): 606.2.

### EXAMPLE 2

### Preparation of Compound 2

Compound 2 was prepared in a manner similar to that described in Example 1.

LC-MS (M+H): 528.3.

### EXAMPLE 3

### Preparation of Compound 3

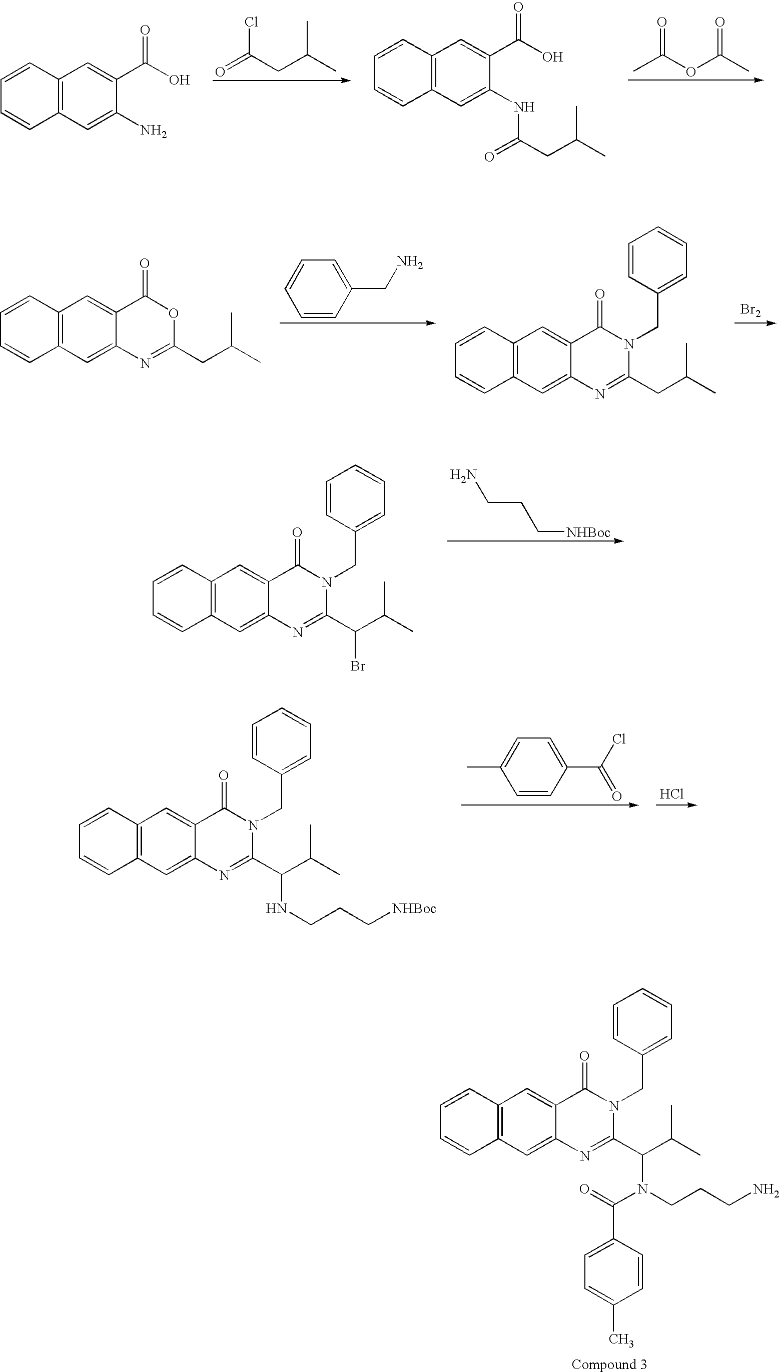

To a stirred solution of 3-amino-2-naphthoic acid (10.0 g, 53.4 mmol) in dry DMF (107 mL) was added isovaleryl chloride (7.2 mL 58.8 mmol) dropwise at room temperature. The mixture was stirred at room temperature overnight and then poured into 850 mL of water. The slurry was stirred at room temperature for another hour and the precipitate was collected by filtration. The collected solid was dried under high vacuum to afford 7.8 g of 3-(3-methyl-butyrylamino)-naphthalene-2-carboxylic acid as a brown solid. LC-MS (M+H): 271.8.

A mixture of 3-(3-methyl-butyrylamino)-naphthalene-2-carboxylic acid (10.9 g, 40.1 mmol) in acetic anhydride (80 mL) was heated to reflux in a reaction vessel fitted with a Dean-Stark trap. After the reaction was complete, the mixture was concentrated under vacuo. The resultant residue was triturated with hexane and the precipitate was collected by filtration and dried under high vacuum to form 10.2 g of 2-isobutyl-naphtho[2,3-d][1,3]oxazin-4-one. LC-MS (M+H): 254.0.

To a solution of 2-isobutyl-naphtho[2,3-d][1,3]oxazin-4-one (10.2 g, 40.1 mmol) in 80 mL of toluene was added benzylamine (4.8 mL, 44.1 mmol). The mixture was heated to reflux and stirred for 1 hour. The reaction mixture was concentrated in vacuo and the residue was suspended in ethylene glycol (160 mL). A small amount of sodium hydroxide (0.8 g, 20.1 mmol) was added and the mixture was stirred at 130° C. for 4 hours. The reaction mixture was cooled to room temperature and washed with saturated a NaHCO₃ aqueous solution and extracted with dichloromethane. The organic layer was separated, washed with brine, dried over anhydrous magnesium sulfate, and concentrated in vacuo. The crude product thus obtained was purified by silica gel column chromatography with 50% dichloromethane/hexane to afford 10.7 g of 3-benzyl-2-isobutyl-3H-benzo[g]quinazolin-4-one. LC-MS (M+H): 343. ¹H-NMR (CDCl₃, 300 Hz): δ 1.01 (d, J=2.1 Hz, 3H), 1.03 (d, J=2.4 Hz, 3H), 2.28-2.42 (m, 1H), 2.66 (d, J=7.2 Hz, 2H), 5.45 (s, 1H), 7.18-7.36 (m, 5H), 7.52 (ddd, J₁=8.1 Hz, J₂=6.9 Hz, J₃=1.2 Hz, 1H), 7.60 (ddd, J₁=8.3 Hz, J₂=6.8 Hz, J₃=1.5 Hz, 1H), 7.97 (d, J=8.1 Hz, 1H), 8.05 (d, J=7.8 Hz, 1H), 8.17 (s, 1H), 8.93 (s, 1H).

To a solution of 3-benzyl-2-isobutyl-3H-benzo[g]quinazolin-4-one (3.5 g, 10.2 mmol) and sodium acetate (1.0 g, 12.3 mmol) in acetic acid (102 mL) at 40° C. was added a solution of bromine (0.53 mL 10.2 mmol) in acetic acid (5.3 mL) dropwise via an addition funnel. The mixture was stirred at 40° C. for 2 hours and cooled to room temperature. It was then diluted with water and extracted with dichloromethane. The organic layer was washed with brine, dried over anhydrous magnesium sulfate, and concentrated in vacuo. The resultant solid was washed with hexane and ether to give 4.68 g of the crude 3-benzyl-2-(1-bromo-2-methyl-propyl)-3H-benzo[g]q-uinazolin-4-one. LC-MS (M+H): 421.1.

Tert-butyl N-(3-aminopropyl)carbamate (12.6 mL) was added to a solution of the crude 3-benzyl-2-(1-bromo-2-methyl-propyl)-3H-benzo[g]q-uinazolin-4-one (4.2 g, 8.4 mmol) in acetonitrile (8.4 mL). The mixture was stirred at 60° C. for 6 hours and cooled to room temperature. After the solvent was evaporated, the residue was dissolved in dichloromethane and washed with a saturated NaHCO₃ aqueous solution. The organic layer was separated, washed with brine, dried over anhydrous MgSO₄, and concentrated in vacuo. The crude product thus obtained was purified by silica gel column chromatography with 20% ethyl acetate/1% triethylamine/hexane to give 0.16 g of {3-[1-(3-benzyl-4-oxo-3,4-dihydro-benzo[g]quinazolin-2-yl)-2-methyl-propylamino]-propyl}-carbamic acid tert-butyl ester. LC-MS (M+H): 515.3.

Para-toluoyl chloride (0.018 mL, 0.134 mmol) was added to a solution of {3-[1-(3-benzyl-4-oxo-3,4-dihydro-benzo[g]quinazolin-2-yl)-2-methyl-propylamino]-propyl}-carbamic acid tert-butyl ester (0.053 g, 0.089 mmol) and triethylamine (0.025 mL, 0.178 mmol) in CH₂Cl₂ (0.45 mL) at 0° C. After the mixture was stirred at room temperature overnight, it was diluted with a saturated sodium bicarbonate aqueous solution and extracted with CH₂Cl₂. The organic layer was then separated, washed with brine, dried over anhydrous MgSO₄, and concentrated in vacuo. The crude product thus obtained was purified by silica gel column chromatography with 25% ethyl acetate/hexane to afford 0.047 g of Boc-protected Compound 3. LC-MS (M+H): 633.4, ¹H-NMR (CDCl₃, 300 Hz): 0.46-0.48 (m, 3H), 1.00-1.03 (m, 3H), 1.31 (s, 9H), 1.64-1.80 (m, 2H), 2.42 (s, 3H), 2.51-2.77 (m, 1H), 2.80-3.00 (m, 1H), 3.39-3.50 (m, 1H), 3.56-3.65 (m, 1H), 3.87 (s, 1H), 5.29 (ABq, J=15.5 Hz, 1H), 5.77 (d, J=10.5 Hz, 1H), 6.25 (ABq, J=15.5 Hz, 1H), 7.26-7.38 (m, 7H), 7.49 (d, J=6.9 Hz, 2H), 7.59-7.70 (m, 2H), 8.05 (d, J=7.8 Hz, 1H), 8.15 (d, J=8.1 Hz, 1H), 8.31 (s, 1H), 9.07 (s, 1H).

The Boc-protected Compound 3 (0.047 g 0.074 mmol) was dissolved in dichloromethane (0.37 mL), followed by the addition of a solution of 4 M HCl in 1,4-dioxane (0.37 mL). The mixture was stirred at room temperature for 2 hours and the organic solvent was evaporated under vacuum. The resultant solid was washed with ether and dried under high vacuum to give 0.038 g of the hydrochloride salt of Compound 3. LC-MS (M+H): 533.3.

### EXAMPLES 4-9

### Preparation of Compounds 4-9

Compounds 4-9 were prepared in a manner similar to that described in Example 3. Their analytical data are provided below.

Compound 4: LC-MS (M+H): 613.2.

Compound 5: LC-MS (M+H): 573.2.

Compound 6: LC-MS (M+H): 653.2.

Compound 7: LC-MS (M+H): 519.4.

Compound 8: LC-MS (M+H): 598.2.

Compound 9: LC-MS (M+H): 659.1.

### EXAMPLE 10

### Preparation of Compound 10

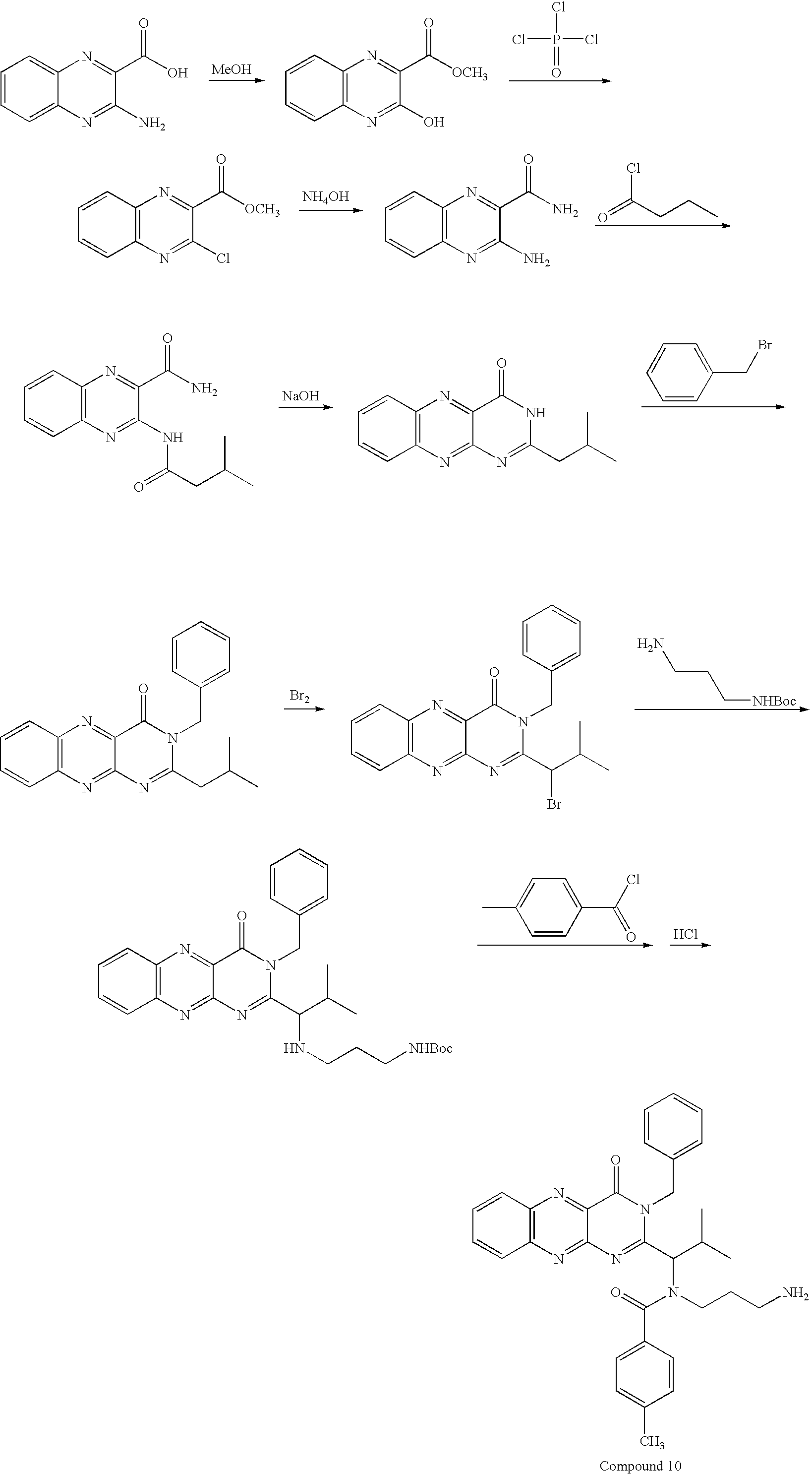

H₂SO₄ (16 mL) was added dropwise to a stirred solution of 3-hydroxy-2-quinoxalinecarboxylic acid (9.3 g, 49.0 mmol) in methanol (245 mL) at room temperature. After the mixture was stirred at room temperature overnight, the methanol was removed under vacuum. The residue thus obtained was dissolved in ethyl acetate and washed with water. The organic layer was separated, washed with brine, dried over anhydrous MgSO₄, and concentrated in vacuo to afford 8.03 g of crude methyl 3-hydroxyquinoxaline-2-carboxylate as a light orange solid. LC-MS (M+H): 205.0.

Methyl 3-hydroxyquinoxaline-2-carboxylate (8.2 g, 40.1 mmol) was stirred in phosphoryl chloride (100 mL) at refluxing temperature for 3 hours. The reaction mixture was then cooled to room temperature and poured into ice water. The slurry was stirred for 1 hour and treated with ammonia till pH was 7˜8. The precipitate thus formed was isolated by filtration to give 7.75 g of crude methyl 3-chloroquinoxaline-2-carboxylate as a white solid. LC-MS (M+H): 223.0.

A suspension solution of methyl 3-chloroquinoxaline-2-carboxylate (5.0 g, 22.47 mmol) in a 28% ammonium hydroxide aqueous solution (112 mL) was stirred at 60° C. overnight. The reaction mixture was cooled to room temperature and filtered to give 2.02 g of 3-aminoquinoxaline-2-carboxamide as a yellow solid. LC-MS (M+H): 189.0.

Isovaleryl chloride (3.2 mL, 25.7 mmol) was added to a stirred solution of 3-aminoquinoxaline-2-carboxamide (2.02 g, 10.71 mmol) and triethylamine (3 mL, 21.4 mmol) in DMF (54 mL) at 0° C. The mixture was then stirred at 60° C. overnight and cooled to room temperature. It was subsequently diluted with a saturated NaHCO₃ aqueous solution and extracted with ethyl acetate. The organic layer was separated, washed with brine, dried over anhydrous MgSO₄, and concentrated in vacuo. The resultant solid was washed with hexane and ether to give 1.78 g of 3-(3-methylbutanamido)quinoxaline-2-carboxamide. LC-MS (M+H): 273.1.

A mixture of 3-(3-methylbutanamido)quinoxaline-2-carboxamide (1.78 g, 6.53 mmol) in ethanol (2.4 mL) and 1N NaOH aqueous solution (2.4 mL) was stirred at room temperature for 1 hour. The precipitate was collected by filtration. After the solution was extracted with CH₂Cl₂, the organic layer was separated, washed with brine, dried over anhydrous MgSO₄, and concentrated in vacuo. The solid was combined with the collected precipitate to give 1.62 g of 2-isobutylbenzo[g]pteridin-4(3H)-one. LC-MS (M+H): 255.0.

Benzyl bromide (1.4 mL, 11.88 mmol) was added to a suspension solution of 2-isobutylbenzo[g]pteridin-4(3H)-one (1.51 g, 5.94 mmol) and potassium carbonate (4.1 g, 29.69 mmol) in acetonitrile (45 mL). The mixture was stirred at 60° C. for 2 hours and cooled to room temperature. The mixture was diluted with a saturated NaHCO₃ aqueous solution and extracted with CH₂Cl₂. The organic layer was collected, washed with brine, dried over anhydrous MgSO₄, and concentrated in vacuo. The residue was purified by silica gel column chromatography with 30% ethyl acetate/1% triethylamine/hexane to give 0.88 g of 3-benzyl-2-isobutylbenzo[g]pteridin-4(3H)-one. LC-MS (M+H): 345.1.

A mixture of 3-benzyl-2-isobutylbenzo[g]pteridin-4(3H)-one (0.88 g, 2.55 mmol) and sodium acetate (0.25 g, 3.06 mmol) was stirred in acetic acid (25 mL) at 40° C. A solution of bromine (0.13 mL, 2.55 mmol) in acetic acid (1.3 mL) was then added dropwise via an addition funnel. The mixture was stirred at 40° C. for 1 hour and cooled to room temperature. It was then diluted with water and extracted with ethyl acetate. The organic layer was collected, washed with brine, dried over anhydrous MgSO₄, and concentrated in vacuo to give 1.06 g of 3-benzyl-2-(1-bromo-2-methyl-propyl)-3H-benzo[g]pteridin-4-one as a yellow solid. LC-MS (M+H): 423.0. ¹H-NMR (CDCl₃, 300 Hz): δ0.61 (d, J=6.6 Hz, 3H), 1.15 (d, J=6.6 Hz, 3H), 2.94-3.07 (m, 1H), 4.52 (d, J=10.2 Hz, 1H), 4.94 (ABq, J=15.9 Hz, 1H), 6.35 (ABq, J=15.9 Hz, 1H), 7.24-7.39 (m, 5H), 7.90 (ddd, J₁=7.7 Hz, J₂=7.7 Hz, J₃=1.2 Hz, 1H), 7.98 (ddd, J₁=7.7 Hz, J₂=7.7 Hz, J₃=1.2 Hz, 1H), 8.29 (d, J=8.7 Hz, 1H), 8.46 (d, J=8.4 Hz, 1H).

A solution of 3-benzyl-2-(1-bromo-2-methyl-propyl)-3H-benzo[g]pteridin-4-one (0.42 g, 1.0 mmol) in tert-butyl N-(3-aminopropyl)carbamate (1.5 mL) was stirred at 70° C. for 0.5 hour. The mixture was cooled to room temperature, diluted with a saturated NaHCO₃ aqueous solution and extracted with CH₂Cl₂. The organic layer was collected, washed with brine, dried over anhydrous MgSO₄, and concentrated in vacuo. The residue was purified by silica gel column chromatography with 30% ethyl acetate/1% triethylamine/CH₂Cl₂ to give 0.43 g of {3-[1-(3-Benzyl-4-oxo-3,4-dihydro-benzo[g]pteridin-2-yl)-2-methyl-propylamino]-propyl}-carbamic acid tert-butyl ester. LC-MS (M+H): 279.0.

para-Toluoyl chloride (0.16 mL, 1.25 mmol) was added to a stirred solution of {3-[1-(3-Benzyl-4-oxo-3,4-dihydro-benzo[g]pteridin-2-yl)-2-methyl-propylamino]-propyl}-carbamic acid tert-butyl ester (0.83 mmol, 0.43 g) and triethylamine (0.23 mL, 1.66 mmol) in CH₂Cl₂ (4.2 mL) at 0° C. After the mixture was stirred at room temperature overnight, it was diluted with a saturated NaHCO₃ aqueous solution and extracted with CH₂Cl₂. The organic layer was washed with brine, dried over anhydrous MgSO₄, and concentrated in vacuo. The crude material was purified by silica gel column chromatography with 60% ethyl acetant/1% triethylamine/hexane to give 0.20 g of the Boc-protected form of Compound 10. LC-MS (M+H): 635.3. ¹H-NMR (CDCl₃, 300 Hz): 0.79 (d, J=6.6 Hz, 3H), 1.26 (d, J=2.1 Hz, 3H), 1.44 (s, 9H), 1.74-1.84 (m, 2H), 2.20 (s, 3H), 3.15-3.30 (m, 2H), 3.49 (s, 1H), 3.55-3.70 (m, 2H), 4.95 (br, 1H), 5.33 (br, 1H), 5.45 (br, 1H), 6.94 (d, J=7.8 Hz, 2H), 7.26-7.40 (m, 7H), 7.63-7.66 (m, 2H), 8.03 (br, 1H), 8.40-8.43 (m, 1H), 9.94 (br, 1H).

A mixture of Boc-protected Compound 10 (0.20 g, 0.32 mmol) and trifluoroacetic acid (1.6 mL) in CH₂Cl₂ (1.6 mL) was stirred at room temperature for 2 hours. After the organic volatiles were removed by evaporation under vacuum, the resultant residue was washed with ether and dried under high vacuum to give 0.20 g of the trifluoroacetic acid salt of Compound 10. LC-MS (M+H): 535.2.

### EXAMPLE 11

### Preparation of Compound 11

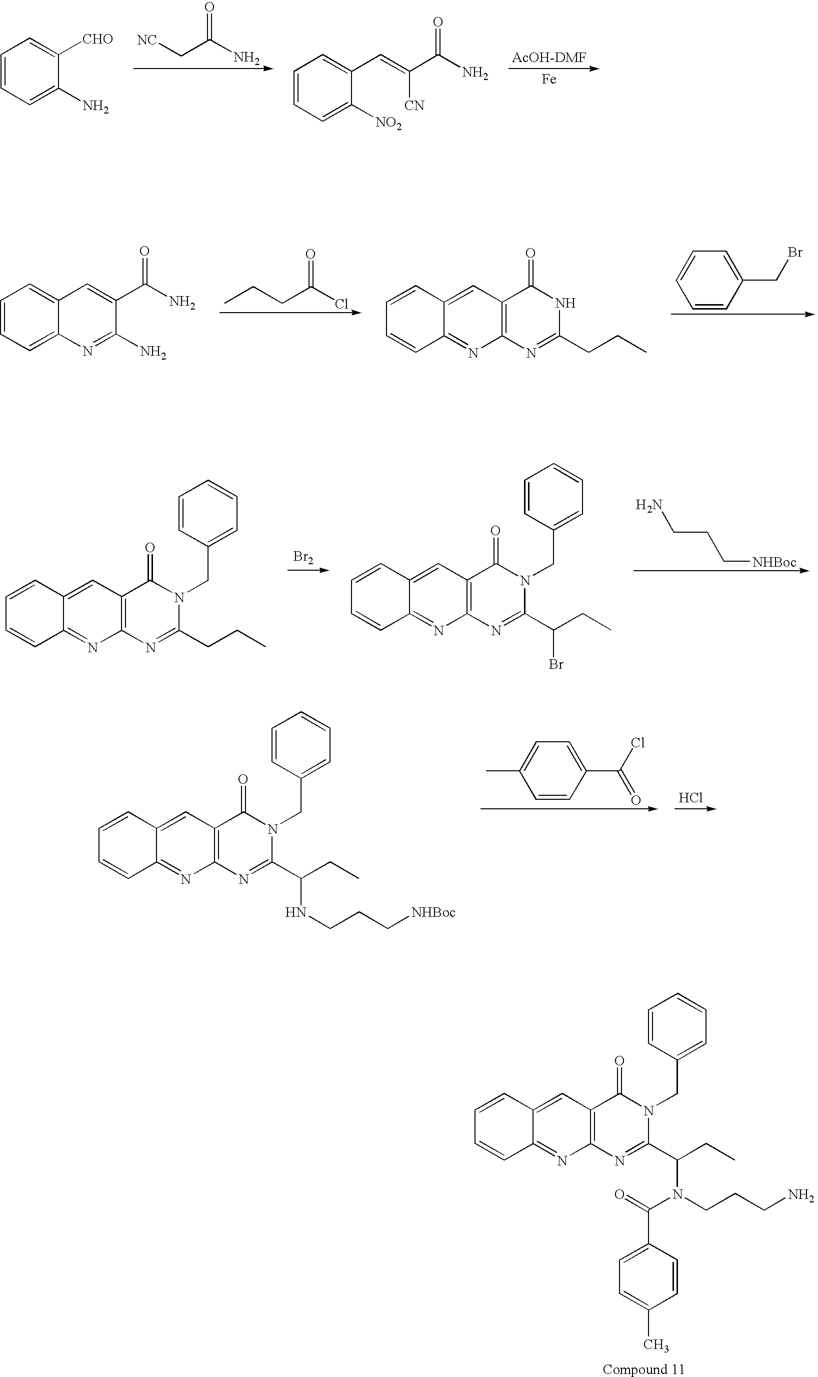

To a solution of 2-nitro-benzaldehyde (10.0 g, 66.2 mmol) in methanol (33 mL) was added piperidine (0.56 g, 6.62 mmol) followed by 2-cyanoacetamide (6.12 g, 72.8 mmol). The mixture was heated under reflux for 2 hours and placed in an ice bath. The precipitate thus formed was washed with cold i-PrOH (100 mL) and dried to give 12.9 g of 2-cyano-3-(2-nitro-phenyl)-acrylamide. LC-MS (M+H): 218.0.

2-Cyano-3-(2-nitro-phenyl)-acrylamide (6.3 g, 29 mmol) and iron powder (7.4 g, 132 mmol) were stirred in a 50% AcOH-DMF (82 mL) solution at 90° C. for 4 hours. The hot mixture was filtered and the dark red filtrate was washed with hot HOAc (17.3 mL). The filtrate was then added to an 1N HCl (173 mL) aqueous solution, which was made alkaline by adding a 10% NaOH aqueous solution. The solution was concentrated in vacuo to afford 3.1 g of crude 2-amino-quinoline-3-carboxylic acid amide, which was used in the next step without further purification. LC-MS (M+H): 188.0.

2-Amino-quinoline-3-carboxylic acid amide (3.1 g, 16.6 mmol) and triethylamine (3.36 g, 33.2 mmol) were dissolved in 1,4-diaxone (83 mL). After the mixture was stirred at 60° C., butyryl chloride (2.65 g, 24.8 mmol) was added and the mixture was stirred overnight. It was then poured an into 1 N NaOH aqueous solution (300 mL) and the resultant slurry was stirred at room temperature for 1 hour. The precipitate thus formed was collected by filtration and dried under vacuum to afford 1.6 g of 2-propyl-3H-pyrimido[4,5-b]quinolin-4-one. LC-MS (M+H): 240.0.

2-Propyl-3H-pyrimido[4,5-b]quinolin-4-one (1.6 g, 6.69 mmol) and K₂CO₃ (4.6 g, 33.4 mmol) were stirred in DMF (67 mL) at 60° C. for 30 minutes. After benzyl bromide (2.3 g, 13.4 mmol) was added, the mixture was stirred at 60° C. overnight. The mixture was then diluted with ethyl acetate (100 mL) and washed with water (3×50 mL). The organic layer was dried over anhydrous magnesium sulfate and concentrated in vacuo. The crude residue was purified by silica gel column chromatography with 20% ethyl acetate/hexane to give 0.7 g of 3-benzyl-2-propyl-3H-pyrimido[4,5-b]quinolin-4-one. LC-MS (M+H): 330.0.

3-Benzyl-2-propyl-3H-pyrimido[4,5-b]quinolin-4-one (0.42 g, 1.27 mmol) and sodium acetate (0.52 g, 6.36 mmol) were dissolved in 100 mL of glacial acetic acid. After the mixture was stirred at 40° C., a solution of bromine (0.22 g, 1.4 mmol) in glacial acetic acid (5 mL) was added dropwise via an addition funnel over 10 minutes. The reaction mixture was then stirred at 40° C. for 30 minutes and poured into 200 mL of water. After the slurry was stirred at room temperature for 1 hour, the precipitate thus formed was collected by filtration and dried under high vacuum to afford 0.4 g of 3-benzyl-2-(1-bromo-propyl)-3H-pyrimido[4,5-b]quinolin-4-one. LC-MS (M+H): 407.8.

3-Benzyl-2-(1-bromo-propyl)-3H-pyrimido[4,5-b]quinolin-4-one (0.25 g, 0.6 mmol) and tert-butyl N-(3-aminopropyl)carbamate (0.43 g, 2.45 mmol) in EtOH (30 ml) were stirred at 60° C. After 48 hours, the mixture was concentrated, diluted with dichloromethane (10 mL), and washed with a saturated NaHCO₃ aqueous solution (3×10 mL). The organic layer was collected, dried over anhydrous magnesium sulfate, and concentrated in vacuo. The crude was purified by silica gel column chromatography with 50% ethyl acetate/hexane to give 0.18 g of {3-[1-(3-benzyl-4-oxo-3,4-dihydro-pyrimido[4,5-b]quinolin-2-yl)-propylamino]-propyl}-carbamic acid tert-butyl ester. LC-MS (M+H): 502.0.

4-Methylbenzoyl chloride (0.039 g, 0.25 mmol) was added dropwise to a solution of {3-[1-(3-benzyl-4-oxo-3,4-dihydro-pyrimido[4,5-b]quinolin-2-yl)-propylamino]-propyl}-carbamic acid tert-butyl ester (0.068 g, 0.13 mmol) and triethylamine (0.038 g, 0.37 mmol) in dichloromethane (5 mL) at 0° C. The mixture was stirred at room temperature overnight and washed with a saturated NaHCO₃ aqueous solution (10 mL). The organic layer was collected, dried over anhydrous magnesium sulfate, and concentrated under vacuum. The residue was purified by silica gel column chromatography with 20% ethyl acetate/hexane to give 0.018 g of Boc-protected Compound 11. ¹H NMR: δ 9.30 (s, 1H), 8.28 (d, J=8.4 Hz, 1H), 8.04 (d, J=8.4 Hz, 1H), 7.87 (dd, J=7.8, 8.4 Hz, 1H), 7.60 (dd, J=7.8, 8.4 Hz, 1H), 7.33-7.16 (m, 9H), 6.08 (ABq, J=22.8 Hz, 1H), 5.91 (s, 1H), 5.28 (ABq, J=22.8 Hz, 1H), 4.86 (s, 1H), 3.50-3.24 (m, 2H), 2.71 (b, 1H), 2.34 (s, 3H), 2.06-1.38 (m, 4H), 1.29 (s, 9H), 0.68 (b, 3H).

A mixture of the Boc-protected Compound 11 (0.01 g, 0.03 mmol) and a 4 N HCl 1,4-dioxane solution (5 mL) was stirred at room temperature for 4 hours. The solvent was removed by evaporation under vacuum and the solid was washed with ether and dried under high vacuum to give 0.007 g of the hydrochloride salt of Compound 11. LC-MS (M+H): 520.1.

### EXAMPLE 12

### Preparation of Compound 12

Compound 12 were prepared in a manner similar to that described in Example 11.

LC-MS (M+H): 554.0.

### EXAMPLE 13

### Preparation of Compound 13

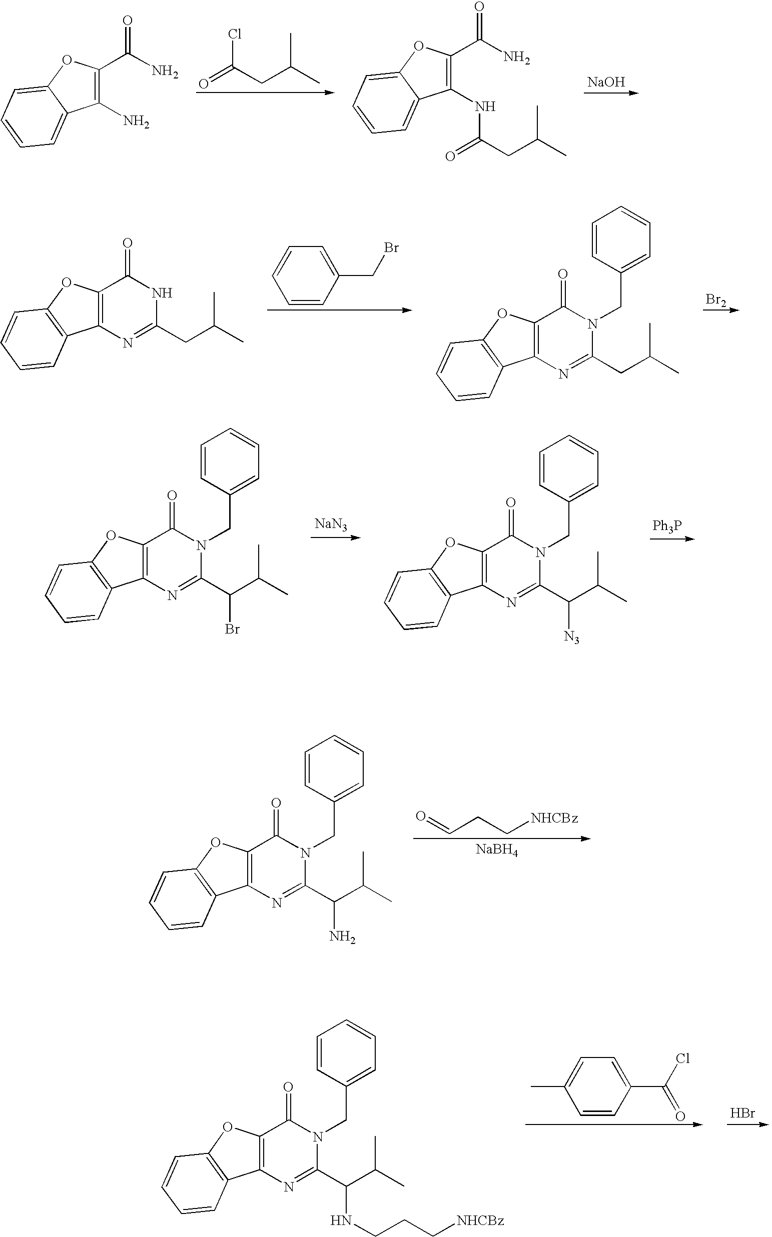

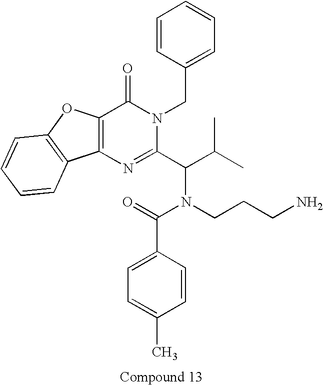

Isovaleryl chloride (4.3 mL, 34.7 mmol) was added dropwise to a stirred solution of 3-aminobenzofuran-2-carboxamide (5.09 g, 28.9 mmol) in 50 mL of dry DMF at room temperature. After the mixture was stirred at room temperature for 4 hours, it was poured into 400 mL of water and stirred for 1 hour. The precipitate formed was collected by filtration and dried under reduced pressure to give 4.90 g of 3-(3-methyl-butyrylamino)-benzofuran-2-carboxylic acid amide. LC-MS (M+H): 261.

To a solution of 3-(3-methyl-butyrylamino)-benzofuran-2-carboxylic acid amide (3.86 g, 14.8 mmol) in 30 mL of EtOH was added 30 mL of an 1 N NaOH aqueous solution. The mixture was stirred at refluxing temperature for 6 hours and treated with an 1 N HCl aqueous solution till pH reaches 7. The precipitate was collected by filtration and dried under reduced pressure to give 3.38 g of 2-isobutylbenzofuro[3,2-d]pyrimidin-4(3H)-one. LC-MS (M+H): 243.1.

To a stirred solution of 2-isobutylbenzofuro[3,2-d]pyrimidin-4(3H)-one (1.02 g, 4.12 mmol) in 30 mL of dried acetonitrile were added potassium carbonate (2.8 g, 20.3 mmol) and benzyl bromide (0.98 mL, 8.24 mmol). The mixture was stirred at 60° C. for 3 hours and concentrated in vacuo. After the resultant residue was dissolved in dichloromethane, the solution was washed with a saturated NaHCO₃ aqueous solution and brine, dried over anhydrous MgSO₄, and concentrated in vacuo. The crude product was purified by silica gel column chromatography with 10% ethyl acetate/hexane to give 0.63 g of 3-benzyl-2-isobutyl-3H-benzo[4,5]furo[3,2-d]pyramidin-4-one. LC-MS (M+H): 333.0.

To a stirred mixture of 3-benzyl-2-isobutyl-3H-benzo[4,5]furo[3,2-d]pyramidin-4-one (0.63 g, 1.88 mmol) and sodium acetate (1.54 g, 18.8 mmol) in 15 mL of glacial acetic acid was added a solution of 0.11 mL bromine (2.1 mmol) in 3.8 mL of glacial acetic acid dropwise over 10 minutes via an addition funnel. The reaction mixture was stirred at 80° C. for 24 hours and poured into 100 mL of water. The slurry was stirred for 1 hour and the precipitate was collected by filtration and dried under reduced pressure to give 0.75 g of 3-benzyl-2-(1-bromo-2-methyl-propyl)-3H-benzo[4,5]furo[3,2-d]-pyrimidin-4-one. ¹H NMR (300 MHz, CDCl₃): δ 8.09 (d, J=7.8, 1H), 7.69 (d, J=8.4, 1H), 7.61 (d, J=7.5, 1H), 7.45 (d, J=7.5, 1H), 7.25-7.37 (m, 3H), 7.17-7.19 (m, 2H), 6.37 (ABq, J=15.9, 1H), 4.90 (ABq, J=15.9, 1H), 4.52 (d, J=9.9, 1H), 2.82-2.94 (m, 1H), 1.14 (d, J=6.6, 3H), 0.56 (d, J=6.6, 3H). LC-MS (M+H): 412.3.

A mixture of 3-benzyl-2-(1-bromo-2-methyl-propyl)-3H-benzo[4,5]furo[3,2-d]-pyrimidin-4-one (0.75 g, 82 mmol) and sodium azide (0.18 g, 2.73 mmol) in 18.2 mL of dry DMF was stirred at 60° C. for 3 hours. After ethyl acetate was added, the solution was washed with water and brine, dried over anhydrous MgSO₄, and concentrated in vacuo to give 0.64 g of 2-(1-azido-2-methyl-propyl)-3-benzyl-3H-benzo[4,5]-furo[3,2-d]pyrimidin-4-one. LC-MS (M+H): 374.0.

0.05 mL of water was added to a stirred solution of 2-(1-azido-2-methyl-propyl)-3-benzyl-3H-benzo[4,5]-furo[3,2-d]pyrimidin-4-one (0.64 g, 1.72 mmol) and triphenylphosphine (0.45 g, 1.72 mmol) in 8.6 mL of THF. After the mixture was stirred at room temperature overnight, the mixture was concentrated in vacuo and the resultant crude product was purified by silica gel column chromatography with 30% ethyl acetate/hexane to give 0.44 g of 2-(1-amino-2-methyl-propyl)-3-benzyl-3H-benzo[4,5]furo[3,2-d]-pyrimidin-4-one. LC-MS (M+H): 348.5.

0.06 mL of HOAc (1.04 mmol) was added to a mixture of 2-(1-amino-2-methyl-propyl)-3-benzyl-3H-benzo[4,5]furo[3,2-d]-pyrimidin-4-one (0.18 g, 0.52 mmol) and 3-[(benzyloxycarbonyl)amino]-1-propanal (0.21 g, 1.04 mmol) in 3.0 mL of MeOH. The mixture was stirred at room temperature for 1 hour and cooled to 0° C. in an ice batch. After sodium borohydride (0.025 g, 0.66 mmol) was added, the reaction mixture was slowly warmed up and stirred at room temperature for 18 hours. The mixture was concentrated in vacuo and the residue was dissolved in dichloromethane. The dichloromethane solution was washed with a NaHCO₃ aqueous solution and brine, dried over anhydrous MgSO₄, and concentrated in vacuo. The crude product was purified by silica gel chromatography with 25% ethyl acetate/hexane to give 0.10 g of {3-[1-(3-benzyl-4-oxo-3,4-dihydro-benzo[4,5]furo[3,2-d]pyrimidin-2-yl)-2-methyl-propylamino]-propyl}-carbamic acid benzyl ester. LC-MS (M+H): 539.2.

4-Methylbenzoyl chloride (0.04 mL, 0.27 mmol) was added dropwise at 0° C. to a stirred solution of {3-[1-(3-benzyl-4-oxo-3,4-dihydro-benzo[4,5]furo[3,2-d]pyrimidin-2-yl)-2-methyl-propylamino]-propyl}-carbamic acid benzyl ester (0.10 g, 0.18 mmol) and triethylamine (0.05 mL, 0.36 mmol) in 2.0 mL of dry dichloromethane. After the mixture was stirred at room temperature for 19 hours, it was concentrated in vacuo and the residue was dissolved in dichloromethane. The organic layer was washed with brine, dried over anhydrous MgSO₄, and concentrated in vacuo. The crude product was purified by silica gel chromatography with 15% ethyl acetate/hexane to give 0.06 g of CBz-protected Compound 13. ¹H NMR: δ 7.97 (d, J=7.8, 1H), 7.93 (d, J=7.8, 1H), 7.62 (d, J=8.4, 1H), 7.52 (t, J=8.4, 1H), 7.40 (d, J=6.9, 2H), 7.13-7.28 (m, 12H), 6.23 (ABq, J=15.6, 1H), 5.75 (d, J=10.8, 1H), 5.27 (ABq, J=15.6, 1H), 4.86-4.95 (m, 2H), 3.97 (s, 1H), 3.42-3.59 (m, 1H), 3.28-3.42 (m, 1H), 2.76-2.79 (m, 1H), 2.62-2.66 (m, 2H), 2.21 (s, 3H), 0.92 (d, J=6.0, 3H), 0.62-0.78 (m, 2H), 0.27 (d, J=6.0, 3H). LC-MS (M+H): 657.3.

To the stirred solution of CBz-protected Compound 13 (0.03 g, 0.05 mmol) in 0.3 mL of dichloromethane was added 0.3 mL of a 33% HBr/HOAc solution dropwise at 0° C. After the mixture was stirred at room temperature for 18 hours, it was concentrated in vacuo and the solid was washed with ether and dried under high vacuum to give 0.023 g of the hydrobromide salt of Compound 13. LC-MS (M+H): 523.3.

The enantiomers of_Compound 13 was isolated as follows: The (−)-enantiomer of Compound 13 was obtained by chiral separation at a retention time of 6.963 minutes on a ChiralPak ODH (Daicel) column using hexane/2-propanol/diethylamine (85/15/0.1 by volume) as an eluant. The (+)-enantiomer of Compound 13 was obtained by chiral separation at a retention time of 40.978 minutes on the same column using the same eluant.

### EXAMPLES 14-149

### Preparation of Compounds 14-149

Compounds 14-149 were prepared in a manner similar to that described in Example 13. Their analytical data are provided below.

Compound 14: LC-MS (M+H): 607.2.

Compound 15: LC-MS (M+H): 529.2.

Compound 16: LC-MS (M+H): 562.7.

Compound 17: LC-MS (M+H): 508.8.

Compound 18: LC-MS (M+H): 574.6.

Compound 19: LC-MS (M+H): 536.8.

Compound 20: LC-MS (M+H): 602.6.

Compound 21: LC-MS (M+H): 494.8.

Compound 22: LC-MS (M+H): 522.8.

Compound 23: LC-MS (M+H): 586.6.

Compound 24: LC-MS (M+H): 616.6.

Compound 25: LC-MS (M+H): 510.3.

Compound 26: LC-MS (M+H): 524.3.

Compound 27: LC-MS (M+H): 541.8.

Compound 28: LC-MS (M+H): 606.6.

Compound 29: LC-MS (M+H): 539.2.

Compound 30: LC-MS (M+H): 590.7.

Compound 31: LC-MS (M+H): 543.2.

Compound 32: LC-MS (M+H): 529.2.

Compound 33: LC-MS (M+H): 579.2.

Compound 34: LC-MS (M+H): 565.2.

Compound 35: LC-MS (M+H): 557.2.

Compound 36: LC-MS (M+H): 543.2.

Compound 37: LC-MS (M+H): 587.2.

Compound 38: LC-MS (M+H): 601.2.

Compound 39: LC-MS (M+H): 523.3.

Compound 40: LC-MS (M+H): 537.3.

Compound 41: LC-MS (M+H): 643.1.

Compound 42: LC-MS (M+H): 628.7.

Compound 43: LC-MS (M+H): 563.0.

Compound 44: LC-MS (M+H): 548.8.

Compound 45: LC-MS (M+H): 596.9.

Compound 46: LC-MS (M+H): 583.1.

Compound 47: LC-MS (M+H): 588.0.

Compound 48: LC-MS (M+H): 574.2.

Compound 49: LC-MS (M+H): 627.1.

Compound 50: LC-MS (M+H): 523.3.

Compound 51: LC-MS (M+H): 509.2.

Compound 52: LC-MS (M+H): 537.3.

Compound 53: LC-MS (M+H): 523.3.

Compound 54: LC-MS (M+H): 552.3.

Compound 55: LC-MS (M+H): 538.2.

Compound 56: LC-MS (M+H): 544.2.

Compound 57: LC-MS (M+H): 538.3.

Compound 58: LC-MS (M+H): 539.3.

Compound 59: LC-MS (M+H): 587.8.

Compound 60: LC-MS (M+H): 527.2.

Compound 61: LC-MS (M+H): 541.2.

Compound 62: LC-MS (M+H): 527.1.

Compound 63: LC-MS (M+H): 541.1.

Compound 64: LC-MS (M+H): 547.2.

Compound 65: LC-MS (M+H): 591.1.

Compound 66: LC-MS (M+H): 613.2.

Compound 67: LC-MS (M+H): 517.2.

Compound 68: LC-MS (M+H): 587.1.

Compound 69: LC-MS (M+H): 612.1.

Compound 70: LC-MS (M+H): 627.2.

Compound 71: LC-MS (M+H): 581.2.

Compound 72: LC-MS (M+H): 572.2.

Compound 73: LC-MS (M+H): 625.1.

Compound 74: LC-MS (M+H): 547.2.

Compound 75: LC-MS (M+H): 533.2.

Compound 76: LC-MS (M+H): 547.2.

Compound 77: LC-MS (M+H): 549.2.

Compound 78: LC-MS (M+H): 563.2.

Compound 79: LC-MS (M+H): 545.3.

Compound 80: LC-MS (M+H): 559.3.

Compound 81: LC-MS (M+H): 595.1.

Compound 82: LC-MS (M+H): 610.8.

Compound 83: LC-MS (M+H): 558.2.

Compound 84: LC-MS (M+H): 547.2.

Compound 85: LC-MS (M+H): 561.2.

Compound 86: LC-MS (M+H): 585.2.

Compound 87: LC-MS (M+H): 599.2.

Compound 88: LC-MS (M+H): 590.8.

Compound 89: LC-MS (M+H): 604.8.

Compound 90: LC-MS (M+H): 550.8.

Compound 91: LC-MS (M+H): 564.8.

Compound 92: LC-MS (M+H): 596.7.

Compound 93: LC-MS (M+H): 610.8.

Compound 94: LC-MS (M+H): 563.2.

Compound 95: LC-MS (M+H): 576.8.

Compound 96: LC-MS (M+H): 560.8.

Compound 97: LC-MS (M+H): 574.9.

Compound 98: LC-MS (M+H): 575.9.

Compound 99: LC-MS (M+H): 612.7.

Compound 100: LC-MS (M+H): 567.2.

Compound 101: LC-MS (M+H): 601.2.

Compound 102: LC-MS (M+H): 575.2.

Compound 103: LC-MS (M+H): 612.8.

Compound 104: LC-MS (M+H): 541.2.

Compound 105: LC-MS (M+H): 527.0.

Compound 106: LC-MS (M+H): 541.0.

Compound 107: LC-MS (M+H): 543.0.

Compound 108: LC-MS (M+H): 557.0.

Compound 109: LC-MS (M+H): 547.2.

Compound 110: LC-MS (M+H): 567.1.

Compound 111: LC-MS (M+H): 542.1.

Compound 112: LC-MS (M+H): 557.9.

Compound 113: LC-MS (M+H): 560.0.

Compound 114: LC-MS (M+H): 590.0.

Compound 115: LC-MS (M+H): 604.0.

Compound 116: LC-MS (M+H): 604.0.

Compound 117: LC-MS (M+H): 532.2.

Compound 118: LC-MS (M+H): 574.0.

Compound 119: LC-MS (M+H): 602.0.

Compound 120: LC-MS (M+H): 639.9.

Compound 121: LC-MS (M+H): 614.2.

Compound 122: LC-MS (M+H): 560.9.

Compound 123: LC-MS (M+H): 541.0.

Compound 124: LC-MS (M+H): 606.1.

Compound 125: LC-MS (M+H): 533.1.

Compound 126: LC-MS (M+H): 533.1.

Compound 127: LC-MS (M+H): 517.2.

Compound 128: LC-MS (M+H): 517.2.

Compound 129: LC-MS (M+H): 574.1.

Compound 130: LC-MS (M+H): 557.2.

Compound 131: LC-MS (M+H): 548.1.

Compound 132: LC-MS (M+H): 545.2.

Compound 133: LC-MS (M+H): 551.2.

Compound 134: LC-MS (M+H): 591.2.

Compound 135: LC-MS (M+H): 539.2.

Compound 136: LC-MS (M+H): 557.3.

Compound 137: LC-MS (M+H): 575.2.

Compound 138: LC-MS (M+H): 579.1.

Compound 139: LC-MS (M+H): 577.2.

Compound 140: LC-MS (M+H): 621.1.

Compound 141: LC-MS (M+H): 644.7.

Compound 142: LC-MS (M+H): 597.2.

Compound 143: LC-MS (M+H): 599.1.

Compound 144: LC-MS (M+H): 625.1.

Compound 145: LC-MS (M+H): 688.7

Compound 146: LC-MS (M+H): 643.1

Compound 147: LC-MS (M+H): 645.1

Compound 148: LC-MS (M+H): 544.9

Compound 149: LC-MS (M+H): 625.5

### EXAMPLE 150

### Preparation of Compound 150

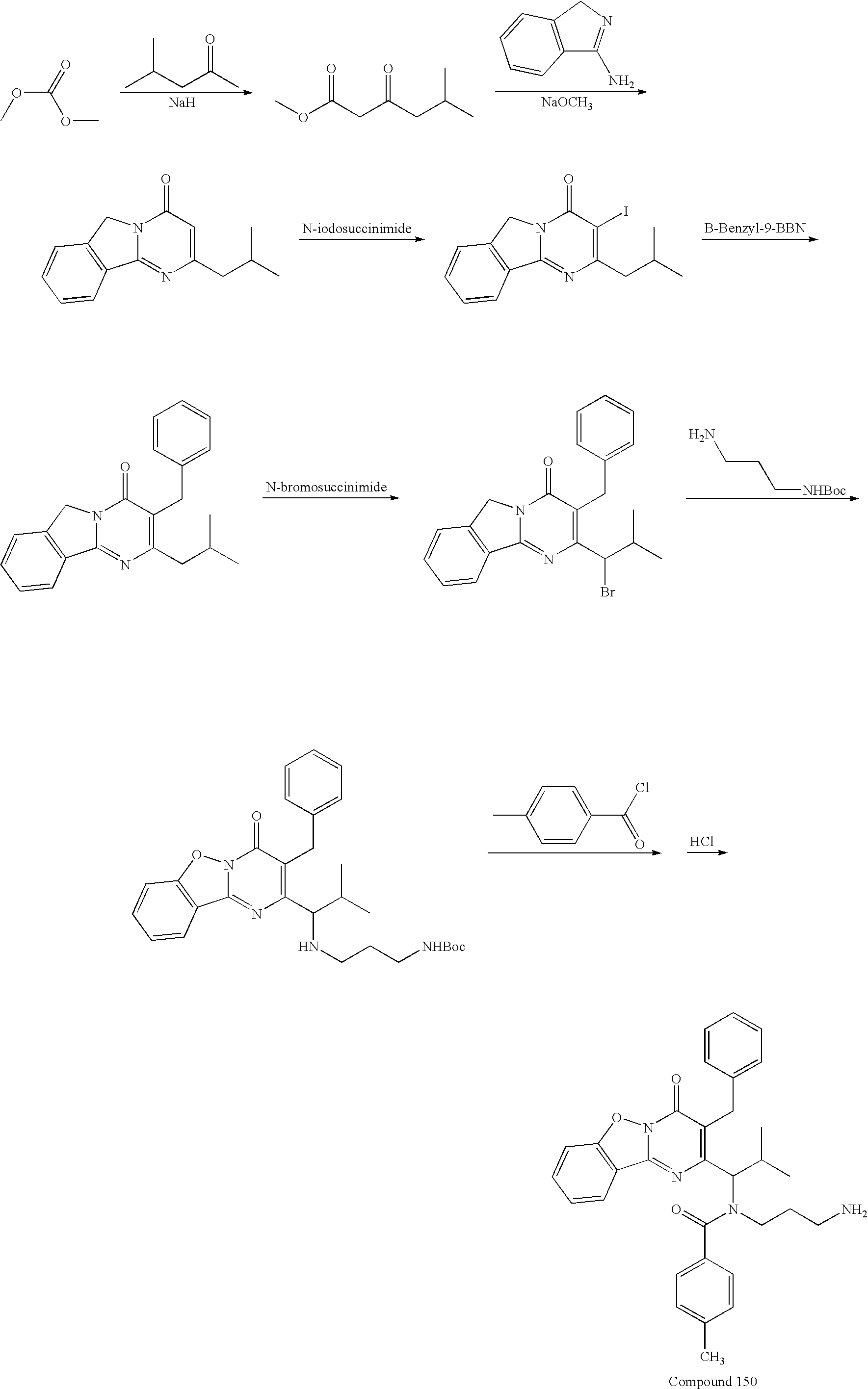

Sodium hydride (4.8 g, 120 mmol) was added in one portion in dimethyl carbonate (25.3 mL, 300 mmol) at 0° C., followed by dropwise addition of 4-methyl-2-pentanone (12.5 mL, 100 mmol) at room temperature over 30 minutes. The mixture was stirred at room temperature overnight. After ethanol (6 mL) was added, the mixture was poured into water (300 mL). The resultant solution was treated with a 3 N HCl aqueous solution to pH 2-3 and extracted with ether. The organic layer was washed with water, a saturated NaHCO₃ aqueous solution, and brine, dried over anhydrous MgSO₄, and concentrated in vacuo to give 13.4 g of 5-methyl-3-oxo-hexanoic acid methyl ester as a light brown oil.

To a solution of 3-amino-1H-isoindole hydrochloride (3.25 g, 19.3 mmol) and 5-methyl-3-oxo-hexanoic acid methyl ester (3.35 g, 21.2 mmol) in MeOH (96 mL) was added a 30 wt % sodium methoxide solution in methanol (7.2 mL, 38.5 mmol). The mixture was stirred at room temperature overnight. The volatiles were evaporated and the solid material was washed with hexane/ether to give 3.4 g of 2-isobutylpyrimido[2,1-a]isoindol-4(6H)-one. LC-MS (M+H): 241.1.

A mixture of 2-isobutylpyrimido[2,1-a]isoindol-4(6H)-one (3.4 g, 14.1 mmol) and N-iodosuccinimide (3.0 g 13.4 mmol) in CH₃CN (46 mL)/CH₂Cl₂ (24 mL) was stirred at 80° C. overnight. After the volatiles were evaporated, the residue was dissolved in dichloromethane and extracted with water. The organic layer was washed with a saturated Na₂S₂O₃ aqueous solution, a saturated NaHCO₃ aqueous solution, and brine, dried over anhydrous MgSO₄, and concentrated in vacuo. The residue was purified by silica gel column chromatography with 30% ethyl acetate/hexane to give 0.49 g of 3-iodo-2-iso-butylpy-rimido[2,1-a]isoindol-4(6H)-one. LC-MS (M+H): 367.0.

A mixture of [1,1′-bis(diphenylphosphino)ferocene]dichloropalladium(II) complex with dichlorocomethane (0.17 g, 0.21 mmol), K₃PO₄ (1.35 g, 6.3 mmol.), 3-iodo-2-iso-butylpy-rimido[2,1-a]isoindol-4(6H)-one (0.56 g, 2.1 mmol), and B-Benzyl-9-BBN (8.5 mL, 4.2 mmol) in DMF (5.3 mL) was stirred at 60° C. overnight. The mixture was then cooled to room temperature, diluted with a 2N NaOH aqueous solution, and extracted with ethyl acetate. The organic layer was collected, washed with brine, dried over anhydrous MgSO₄, and concentrated in vacuo. The crude material was purified silica gel column chromatography with 20% ethyl acetate/hexane to give 0.35 g of 3-benzyl-2-isobutyl-pyrimido[2,1-a]isoindol-4(6H)-one. LC-MS (M+H): 331.1.

A mixture of 3-benzyl-2-isobutyl-pyrimido[2,1-a]isoindol-4(6H)-one (0.35 g 1.05 mmol), N-bromosuccinimide (0.18 g 1.0 mmol), and trifluoroacetic acid (0.008 mL, 0.11 mmol) in CH₃CN (3.6 mL) and CH₂Cl₂ (1.8 mL) was stirred at room temperature for 3 hours. The organic volatiles were removed by evaporation under vacuum. The resultant residue was dissolved in CH₂Cl₂ and treated with a saturated NaHCO₃ aqueous solution and brine, dried over anhydrous MgSO₄, and concentrated in vacuo. The residue was purified by silica gel column chromatography with 20% ethyl acetate/hexane to give 0.13 g of 3-benzyl-2-(1-bromo-2-methylpropyl)pyrimido[2,1-a]isoindol-4-(6-H)-one. LC-MS (M+H): 409.0. ¹H-NMR (CDCl₃, 300 Hz): δ0.58 (d, J=6.9 Hz, 3H), 1.20 (d, J=6.3 Hz, 3H), 2.68-2.80 (m, 1H), 3.84 (ABq, J=15.1 Hz, 1H), 4.32 (ABq, J=15.1 Hz, 1H), 4.79 (d, J=10.2 Hz, 1H), 5.11 (s, 2H), 7.16-7.20 (m, 1H), 7.26-7.28 (m, 5H), 7.54-7.61 (m, 1H), 7.63 (s, 1H), 7.64 (d, J=4.2 Hz, 1H), 8.13 (d, J=7.5 Hz, 1H).

A mixture of 3-benzyl-2-(1-bromo-2-methylpropyl)pyrimido[2,1-a]isoindol-4-(6-H)-one (0.12 g, 0.29 mmol) in tert-butyl N-(3-aminopropyl)carbamate (0.6 mL) was stirred at 70° C. for 1 hour. The mixture was cooled to room temperature, diluted with a saturated NaHCO₃ aqueous solution, and extracted with CH₂Cl₂. The organic layer was collected, washed with brine, dried over anhydrous MgSO₄, and concentrated in vacuo. The residue was purified by silica gel column chromatography with 40% ethyl acetate/1% triethylamine/hexane to give 0.092 g of tert-butyl 3-(1-(3-benzyl-4-oxo-4,6-dihydropyrimido[2,1-a]isoindol-2-yl)-2-methylpropylamino)propylcarbamate. LC-MS (M+H): 503.3.

To a solution of tert-butyl 3-(1-(3-benzyl-4-oxo-4,6-dihydropyrimido[2,1-a]isoindol-2-yl)-2-methylpropylamino)propylcarbamate (0.092 g, 0.18 mmol) and triethylamine (0.05 mL, 0.37 mmol) in CH₂Cl₂ (0.9 mL) was added para-toluoyl chloride (0.04 mL, 0.28 mmol) at 0° C. The mixture was stirred at room temperature overnight, treated with a saturated NaHCO₃ aqueous solution, and extracted with CH₂Cl₂. The organic layer was washed with brine, dried over anhydrous MgSO₄, and concentrated in vacuo. The residue was purified by silica gel column chromatography with 40% ethyl acetate/1% triethylamine/hexane to give 0.033 g of the Boc-protected Compound 145. LC-MS (M+H): 621.4. ¹H-NMR (CDCl₃, 300 Hz): δ0.43 (d, J=6.3 Hz, 2H), 1.05 (d, J=6.3 Hz, 2H), 1.34 (s, 9H), 2.36 (s, 3H), 2.60-2.70 (m, 2H), 2.72-2.82 (m, 1H), 3.36-3.49 (m, 1H), 3.49-3.56 (m, 1H), 3.73-3.83 (m, 1H), 4.20 (ABq, J=14.4 Hz, 1H), 4.40 (d, J=14.4 Hz, 1H), 5.12 (s, 1H), 5.15 (s, 2H), 5.87 (d, J=10.8 Hz, 2H), 7.08-7.29 (m, 8H), 7.45 (d, J=7.2 Hz, 1H), 7.53-7.62 (m, 1H), 7.65 (d, J=4.8 Hz, 2H), 8.07 (d, J=7.8 Hz, 1H).

A mixture of the Boc-protected Compound 150 (0.033 g, 0.053 mmol) and 4M HCl in 1,4-dioxane solution (0.27 mL) in CH₂Cl₂ (0.27 mL) was stirred at room temperature for 2 hours. After the organic solvent was evaporated under vacuum, the resultant residue was washed with ether and dried under high vacuum to give 0.028 g of the hydrochloride salt of Compound 150. LC-MS (M+H): 521.3.

### EXAMPLES 151-155

### Preparation of Compounds 151-155

Compounds 151-155 were prepared in a manner similar to that described in Example 150. Their analytical data are provided below.

Compound 151: LC-MS (M+H): 541.2.

Compound 152: LC-MS (M+H): 539.3.

Compound 153: LC-MS (M+H): 561.3.

Compound 154: LC-MS (M+H): 557.3.

Compound 155: LC-MS (M+H): 585.1.

### EXAMPLE 156

### Preparation of Compound 156

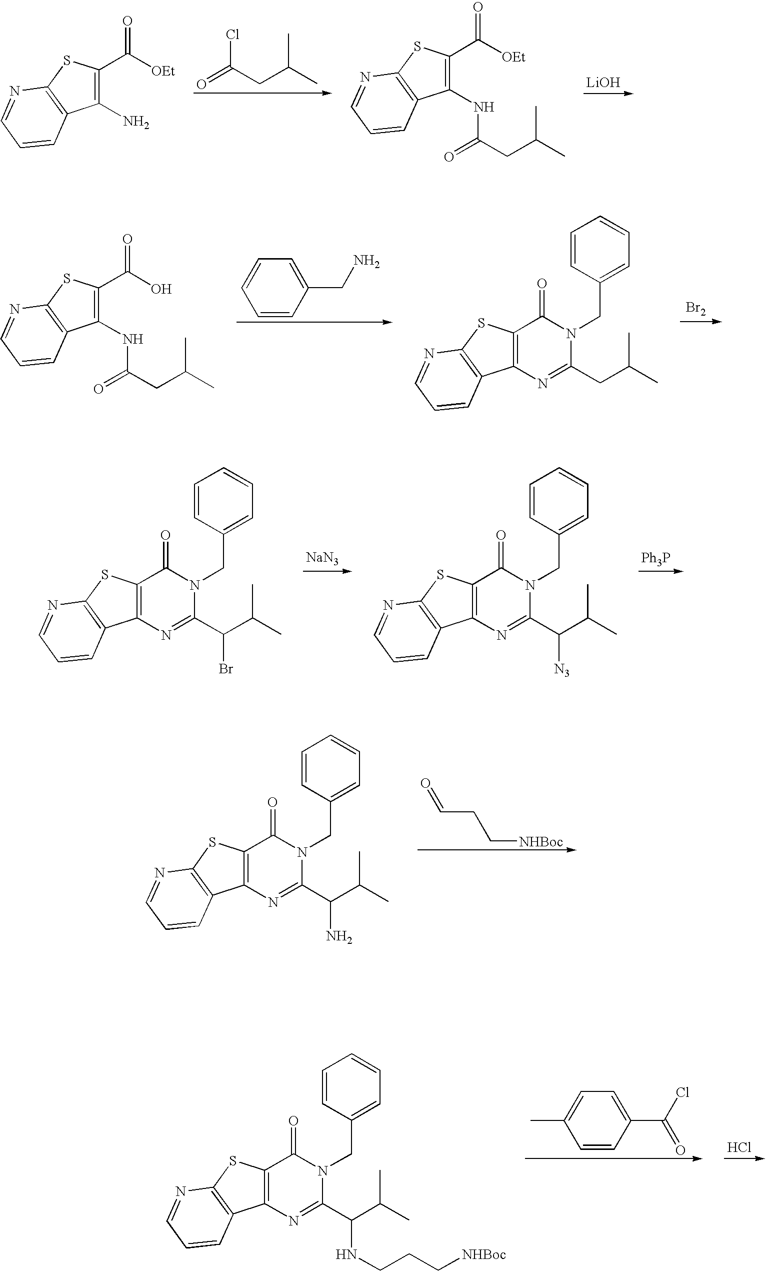

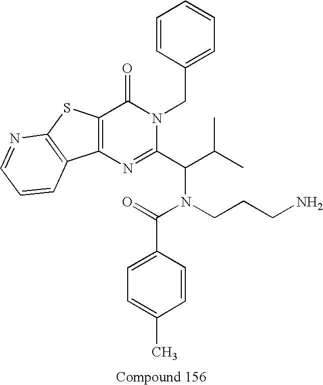

Isovaleryl chloride (3.3 mL, 27.03 mmol) was added dropwise to a stirred solution of ethyl 3-aminothieno[2,3-b]pyridine-2-carboxylate (5.01 g, 22.5 mmol) in 23.0 mL of dry DMF at room temperature. The mixture was stirred at room temperature for 2 hours and poured into 400 mL of water. After the slurry was stirred for 1 hour, the precipitate thus obtained was collected by filtration and dried under reduced pressure to give 6.10 g of ethyl 3-(3-methylbutanamido)thieno[2,3-b]pyridine-2-carboxylate. LC-MS (M+H): 307.1.

24 mL of an 1 N LiOH aqueous solution was added to the solution of ethyl 3-(3-methylbutanamido)thieno[2,3-b]pyridine-2-carboxylate (6.10 g, 19.93 mmol) in 50 mL of THF. The mixture was stirred at 60° C. for 2 hour and concentrated in vacuo. The residue was dissolved in water and treated with a 1 N HCl aqueous solution until pH reached 3-4. The precipitate thus formed was collected by filtration and dried under reduced pressure to give 4.40 g of 3-(3-methylbutanamido)thieno[2,3-b]pyridine-2-carboxylic acid. LC-MS (M+H): 279.1.

To a stirred solution of 3-(3-methylbutanamido)thieno[2,3-b]pyridine-2-carboxylic acid (3.0 g, 3.38 mmol) in 54 mL of dry dichloromethane were added 1,1-ethyl-3-(3-dimethylaminopropyl)carbodiimide hydrochloride salt (EDC, 4.14 g, 21.6 mmol), N-hydroxybenzotriazole (HOBt, 2.91 g, 21.6 mmol), benzyl amine (1.17 g, 0.05 mmol), and N-Methylmorpholine (NMM, 5.8 mL, 50.4 mmol). The mixture was stirred at room temperature for 2 hours and concentrated in vacuo. After the resultant residue was dissolved in dichloromethane, the solution was washed with a saturated NaHCO₃ aqueous solution and brine, dried over anhydrous MgSO₄, and concentrated in vacuo. The residue was purified by silica gel column chromatography with 15% ethyl acetate/hexane to give 0.63 g of 7-benzyl-6-isobutyl-7H-9-thia-1,5,7-triaza-fluoren-8-one. LC-MS (M+H): 350.1.

A mixture of 7-benzyl-6-isobutyl-7H-9-thia-1,5,7-triaza-fluoren-8-one (0.58 g, 1.66 mmol) and sodium acetate (1.36 g, 16.6 mmol) in 13 mL of glycial acetic acid was added a solution of bromine (0.101 mL, 1.99 mmol) in 3.0 mL glacial acetic acid dropwise over 10 minutes via an addition funnel. The reaction mixture was stirred at 80° C. for 46 hours and poured into 50 mL of water. After the slurry was stirred for 1 hour, the precipitate was collected by filtration and dried under reduced pressure to give 0.61 g of 7-benzyl-6-(1-bromo-2-methyl-propyl)-7H-9-thia-1,5,7-triaza-fluoren-8-one. ¹H NMR (300 MHz, CDCl₃): δ 8.80 (d, J=3.3, 1H), 8.58 (d, J=7.5, 1H), 7.49 (dd, J=7.8, 4.8, 1H), 7.30-7.38 (m, 3H), 7.20 (d, J=6.9, 2H), 6.34 (ABq, J=16.2, 1H), 4.91 (ABq, J=16.2, 1H), 4.54 (d, J=9.9, 1H), 2.80-2.92 (m, 1H), 1.16 (d, J=6.6, 3H), 0.59 (d, J=6.6, 3H). LC-MS (M+H): 428.0.

A mixture of 7-benzyl-6-(1-bromo-2-methyl-propyl)-7H-9-thia-1,5,7-triaza-fluoren-8-one (0.61 g, 1.43 mmol) and sodium azide (0.14 g, 2.14 mmol) in 14.3 mL of DMF was stirred at 60° C. for 1 hour. The mixture was diluted with ethyl acetate and washed with brine, dried over anhydrous MgSO₄, and concentrated in vacuo to give 0.54 g of 6-(1-azido-2-methyl-propyl)-7-benzyl-7H-9-thia-1,5,7-triaza-fluoren-8-one. LC-MS (M+H): 390.9.

Water (0.01 mL) was added to a mixture of 6-(1-azido-2-methyl-propyl)-7-benzyl-7H-9-thia-1,5,7-triaza-fluoren-8-one (0.18 g, 0.467 mmol) and triphenylphosphine (0.12 g, 0.46 mmol) in 2.3 mL of THF. The reaction mixture was stirred at room temperature for 16 hours and concentrated in vacuo. The residue was purified by silica gel column chromatography with 30% ethyl acetate/hexane to give 0.14 g of 6-(1-amino-2-methyl-propyl)-7-benzyl-7H-9-thia-1,5,7-triaza-fluoren-8-one. LC-MS (M+H): 364.9.

HOAc (0.05 mL) was added to a stirred solution of 6-(1-amino-2-methyl-propyl)-7-benzyl-7H-9-thia-1,5,7-triaza-fluoren-8-one (0.14 g, 0.38 mmol) and tert-butyl 3-oxopropylcarbamate (0.27 g, 1.54 mmol) in 2.0 mL of MeOH. The mixture was stirred at room temperature for 1 hour and cooled to 0° C. After sodium borohydride (0.02 g, 0.46 mmol) was added in portions, the mixture was stirred at room temperature for 24 hours. The mixture was concentrated in vacuo and the crude material was dissolved in dichloromethane. The solution was washed with 1 mL of a NH₄OH aqueous solution and brine, dried over anhydrous MgSO₄, and concentrated in vacuo. The residue was by silica gel column chromatography with 25% ethyl acetate/hexane to give 0.05 g of {3-[1-(7-benzyl-8-oxo-7,8-dihydro-9-thia-1,5,7-triaza-fluoren-6-yl)-2-methyl-propylamino]-propyl}-carbamic acid tert-butyl ester. LC-MS (M+H): 521.9.

4-Methylbenzoyl chloride (0.02 mL, 0.14 mmol) was added dropwise to a mixture of {3-[1-(7-benzyl-8-oxo-7,8-dihydro-9-thia-1,5,7-triaza-fluoren-6-yl)-2-methyl-propylamino]-propyl}-carbamic acid tert-butyl ester (0.05 g, 0.10 mmol) and triethylamine (0.03 mL, 0.20 mmol) in 0.50 mL of dry dichloromethane at 0° C. The mixture was stirred at room temperature for 18 hours and concentrated in vacuo. After the residue was dissolved in dichloromethane, the solution was washed with brine, dried over anhydrous MgSO₄, and concentrated in vacuo. The residue was purified by silica gel column chromatography with 15% ethyl acetate/hexane to give 0.03 g of Boc-protected Compounds 151. 1HNMR: δ 8.81 (s, 1H), 8.57 (d, J=7.8, 1H), 7.46-7.48 (m, 3H), 7.23-7.35 (m, 7H), 6.26 (ABq, J=14.9, 1H), 5.84 (d, J=10.2, 1H), 5.32 (ABq, J=14.9, 1H), 3.38-3.52 (m, 2H), 2.82-2.92 (m, 1H), 2.57-2.59 (m, 2H), 2.39 (s, 3H), 1.40-1.47 (m, 2H), 1.25 (s, 9H), 0.99 (d, J=6.3, 3H), 0.37 (d, J=6.3, 3H). LC-MS (M+H): 639.9.

To a stirred solution of Boc-protected Compound 156 (0.03 g, 0.05 mmol) in 0.3 mL of dichloromethane was added 0.3 mL of a 4 N HCl dioxane solution dropwise at 0° C. The mixture was slowly warmed up and stirred at room temperature for 3 hours. After it was concentrated in vacuo, the resulting solid was washed with ether and dried to give 0.022 g of Compound 156. LC-MS (M+H): 539.9.

### EXAMPLES 157 AND 158

### Preparation of Compounds 157 and 158

Compounds 157 and 158 were prepared in a manner similar to that described in Example 156. Their analytical data are provided below.

Compound 157: LC-MS (M+H): 601.6.

Compound 158: LC-MS (M+H): 521.8.

### EXAMPLE 159

### Preparation of Compound 159

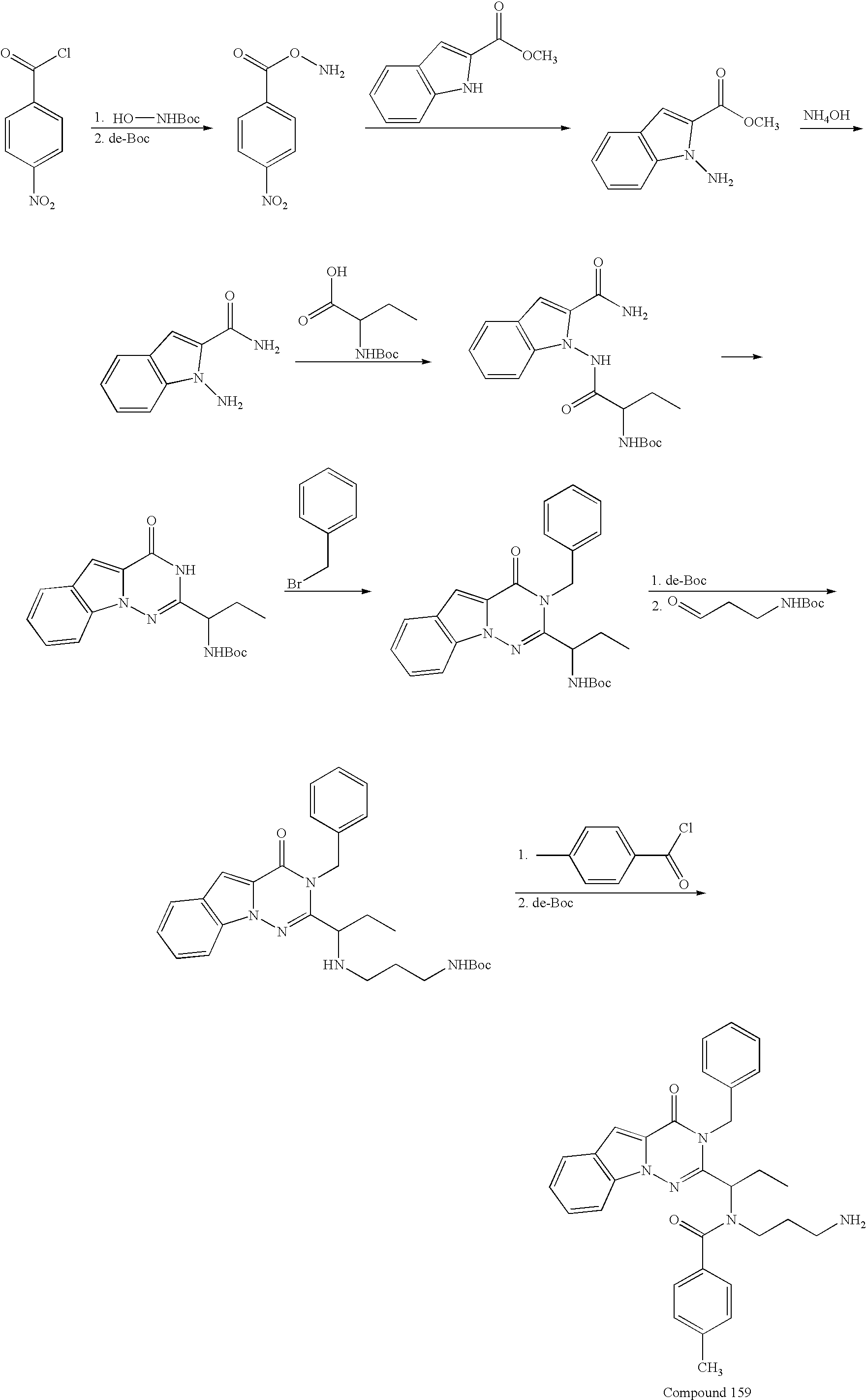

A mixture of 10.00 g (62.1 mmol) of indole-2-carboxylic acid in 300 mL of methanol containing a catalytic amount of sulfuric acid was refluxed until the reaction was complete. After the mixture was concentrated in vacuo, the resultant solid was dissolved in methylene chloride and washed with brine. The organic layer was separated, dried over anhydrous MgSO₄, and concentrated in vacuo to give 10.0 g of one of the starting material indole-2-carboxylic acid methyl ester as an off-white solid. LC-MS (M+H): 175.9.

To a mixture of tert-butyl hydroxycarbamate (9.44 g, 70.9 mmol) and triethylamine (10.8 mL, 78.0 mmol) in CH₂Cl₂ (43 mL) was added 4-nitro-benzoyl chloride (13.16 g, 70.9 mmol) in CH₂Cl₂ (55 mL) dropwise at 0° C. for 40 minutes. The mixture was stirred at 0-5° C. for 5 minutes and warmed up to room temperature in 1.5 hours. After water (62 mL) was added to quench the reaction, the mixture was stirred for another 20 minutes. The organic layer was separated, washed with an 1% NaHCO₃ aqueous solution (48 mL), and treated with methanesulfonic acid (6.9 mL, 106.4 mmol) at room temperature overnight. Hexane (27 mL) was then added and the precipitate was collected by filtration to give O-(4-nitrobenzoyl)hydroxylamine methanesulfonic acid salt. The salt was subsequently dissolved in CH₂Cl₂ (164 mL) and treated with a 6% NaHCO₃ aqueous solution (82 mL). The organic layer was washed with brine, dried over anhydrous MgSO₄, and concentrated. The solid thus obtained was washed with hexane and filtered to give 11.27 g of O-(4-nitrobenzoyl)hydroxylamine as a light yellow solid. LC-MS (M+H): 183.0.

A mixture of methyl 1H-indole-2-carboxylate (9.03 g, 51.6 mmol) and 1.0 M potassium tert-butoxide in THF (62 mL, 61.9 mmol) in 1-methyl-2-pyrrolidinone (67 mL) was stirred at room temperature for 30 minutes. After O-(4-nitrobenzoyl)-hydroxylamine (11.27 g, 61.9 mmol) in 1-methyl-2-pyrrolidinone (45 mL) was added, the mixture was stirred at room temperature for 2 hours, followed by addition of brine and toluene. The organic layer was separated and the aqueous layer was extracted with toluene. The organic layers were then combined, washed with a saturated NaHCO₃ aqueous solution, and concentrated under reduced pressure to give 9.61 g of 1-amino-1H-indole-2-carboxylic acid methyl ester. LC-MS (M+H): 191.1.

A suspension of 1-amino-1H-indole-2-carboxylic acid methyl ester (9.61 g, 50.5 mmol) in a 28% ammonium hydroxide aqueous solution (126 mL) was stirred at 45° C. for 6 hours. The solution was cooled to room temperature and filtered to give 4.94 g of 1-amino-1H-indole-2-carboxamide as a light brown powder. LC-MS (M+H): 176.1.

(2-Boc-amino)butyric acid dicyclohexylamine salt (12.4 g, 32.3 mmol) was dissolved in CH₂Cl₂ (135 mL), followed by the addition of EDC (10.33 g, 53.9 mmol), HOBt (1.82 g, 13.5 mmol), 1-amino-1H-indole-2-carboxamide (4.72 g, 26.9 mmol) and N-methylmorpholine (11.8 mL, 107.8 mmol) at 0° C. The mixture was stirred at room temperature overnight. The mixture was extracted with a saturated NaHCO₃ aqueous solution and diluted with CH₂Cl₂. The organic layer was washed with brine, dried over anhydrous MgSO₄, and concentrated in vacuo. The resultant solid was washed with hexane/ether to give 6.15 g of [1-(2-carbamoyl-indol-1-ylcarbamoyl)-propyl]-carbamic acid tert-butyl ester. LC-MS (M+Na): 383.1.

A mixture of [1-(2-carbamoyl-indol-1-ylcarbamoyl)-propyl]-carbamic acid tert-butyl ester (3.71 g, 10.3 mmol) and potassium hydroxide (2.89 g, 51.5 mmol) in ethylene glcycol (52 mL) was stirred at 85° C. overnight. The mixture was then diluted with CH₂Cl₂ and extracted with water. The organic layer was washed with brine, dried over anhydrous MgSO₄, and concentrated in vacuo to give 0.9 g of the crude [1-(1-oxo-1,2-dihydro-2,4,4a-triaza-fluoren-3-yl)-propyl]-carbamic acid tert-butyl ester. LC-MS (M+H): 343.1. ¹H-NMR (CDCl₃, 300 Hz): δ 1.10 (t, J=7.2 Hz, 3H), 1.46 (s, 9H), 1.96 (m, 1H), 2.17 (m, 1H), 4.50 (m, 1H), 5.09 (m, 1H), 7.30 (dd, J₁=7.2 Hz, J₂=7.2 Hz, 1H), 7.33 (s, 1H), 7.44 (dd, J₁=7.65 Hz, J₂=7.65 Hz, 1H), 7.79 (d, J=8.4 Hz, 1H), 7.92 (d, J=8.4 Hz, 1H), 9.86 (s, 1H).

Benzyl bromide (0.64 mL, 5.41 mmol) was added to a suspension of [1-(1-oxo-1,2-dihydro-2,4,4a-triaza-fluoren-3-yl)-propyl]-carbamic acid tert-butyl ester (1.23 g, 3.60 mmol) and potassium carbonate (2.49 g, 18.02 mmol) in acetonitrile (18 mL). The mixture was stirred at 60° C. for 1 hour and cooled to room temperature. The mixture was diluted with CH₂Cl₂ and extracted with a saturated NaHCO₃ aqueous solution. The organic layer was washed with brine, dried over anhydrous MgSO₄, and concentrated in vacuo. The resultant crude solid was purified on silica gel with 50% CH₂Cl₂/hexane to give 0.43 g of [1-(2-benzyl-1-oxo-1,2-dihydro-2,4,4a-triaza-fluoren-3-yl)-propyl]-carbamic acid tert-butyl ester. LC-MS (M+Na): 455.2.

To [1-(2-benzyl-1-oxo-1,2-dihydro-2,4,4a-triaza-fluoren-3-yl)-propyl]-carbamic acid tert-butyl ester (0.43 g, 0.98 mmol) in 4.9 mL of dichloromethane was added 4.9 mL of 4M HCl in dioxane at 0° C. After the mixture was stirred at room temperature for 4 hours, the organic volatiles were removed by evaporation. The resultant residue was washed with ether and dried under high vacuum to give 0.42 g of the hydrochloride salt of 3-(1-amino-propyl)-2-benzyl-2H-2,4,4a-triaza-fluoren-1-one. LC-MS (M+H):333.1.

A mixture of 3-(1-amino-propyl)-2-benzyl-2H-2,4,4a-triaza-fluoren-1-one hydrochloride salt (0.11 g, 0.27 mmol), triethylamine (0.05 mL, 0.33 mmol), MgSO₄ (0.54 g), and (3-oxo-propyl)-carbamic acid tert-butyl ester (0.06 g, 0.35 mmol) in 1.4 mL of dried dichloromethane was stirred at room temperature. Sodium triacetoxyborohydride (0.069 g, 0.33 mmol) was added into the mixture at 0° C. and the mixture was stirred at room temperature for 2 hours. The mixture was diluted with dichloromethane and washed with ammonium hydroxide solution. The organic layer was washed with brine, dried over anhydrous MgSO₄, and concentrated in vacuo. The crude material was purified by silica gel chromatography with 50% ethyl acetate/hexane to give 0.09 g of {3-[1-(2-benzyl-1-oxo-1,2-dihydro-2,4,4a-triaza-fluoren-3-yl)-propylamino]-propyl}-carbamic acid tert-butyl ester. LC-MS (M+H): 490.3

To a solution of {3-[1-(2-benzyl-1-oxo-1,2-dihydro-2,4,4a-triaza-fluoren-3-yl)-propylamino]-propyl}-carbamic acid tert-butyl ester (0.09 g, 0.18 mmol) and triethylamine (0.05 mL, 0.35 mmol) in 0.9 mL of dried dichloromethane was added p-toluoyl chloride (0.04 mL, 0.26 mmol) at 0° C. The mixture was stirred at room temperature overnight and treated with a saturated NaHCO₃ aqueous solution. The dichloromethane layer was washed with brine, dried over anhydrous MgSO₄, and concentrated in vacuo. The crude material was purified by silica gel chromatography with 25% ethyl acetate/hexane to give 0.09 g of the Boc-protected Compound 159. LC-MS (M+H): 608.0. ¹H-NMR (CDCl₃, 300 Hz): δ 0.88 (t, J=6.6 Hz, 3H), 1.34 (s, 9H), 1.93 (m, 1H), 2.17 (m, 1H), 2.38 (s, 3H), 2.68 (m, 2H), 3.37 (m, 2H), 3.84 (m, 2H), 4.97 (ABq, J=15.5 Hz, 1H), 6.02 (ABq, J=15.5 Hz, 1H), 7.20 (m, 4H), 7.33 (m, 6H), 7.40 (s, 1H), 7.47 (dd, J, =7.5 Hz, J₂=7.5 Hz, 1H), 7.84 (d, J=8.1 Hz, 1H), 7.97 (d, J=8.4 Hz, 1H).

The Boc-protected Compound 159 (0.09 g, 0.15 mmol) in 0.75 mL of dichloromethane was added 0.75 mL of 4M HCl in dioxane at 0° C. After the mixture was stirred at room temperature for 2 hours, the organic volatiles were removed under vacuum. The resultant residue was washed with ether and dried under high vacuum to give 0.07 g of the hydrochloride salt of Compound 159. LC-MS (M+H): 507.9.

### EXAMPLES 160-174

### Preparation of Compounds 160-174

Compounds 160-174 were prepared in a manner similar to that described in Example 13. Their analytical data are provided below.

Compound 160: LC-MS (M+H): 549.2.

Compound 161: LC-MS (M+H): 529.2.

Compound 162: LC-MS (M+H): 533.2.

Compound 163: LC-MS (M+H): 540.2.

Compound 164: LC-MS (M+H): 595.1.

Compound 165: LC-MS (M+H): 577.1.

Compound 166: LC-MS (M+H): 567.2.

Compound 167: LC-MS (M+H): 611.1.

Compound 168: LC-MS (M+H): 565.2.

Compound 169: LC-MS (M+H): 571.1.

Compound 170: LC-MS (M+H): 617.1.

Compound 171: LC-MS (M+H): 605.1.

Compound 172: LC-MS (M+H): 578.1.

Compound 173: LC-MS (M+H): 624.1.

Compound 174: LC-MS (M+H): 576.2.

### EXAMPLE 175

### Preparation of Compound 175

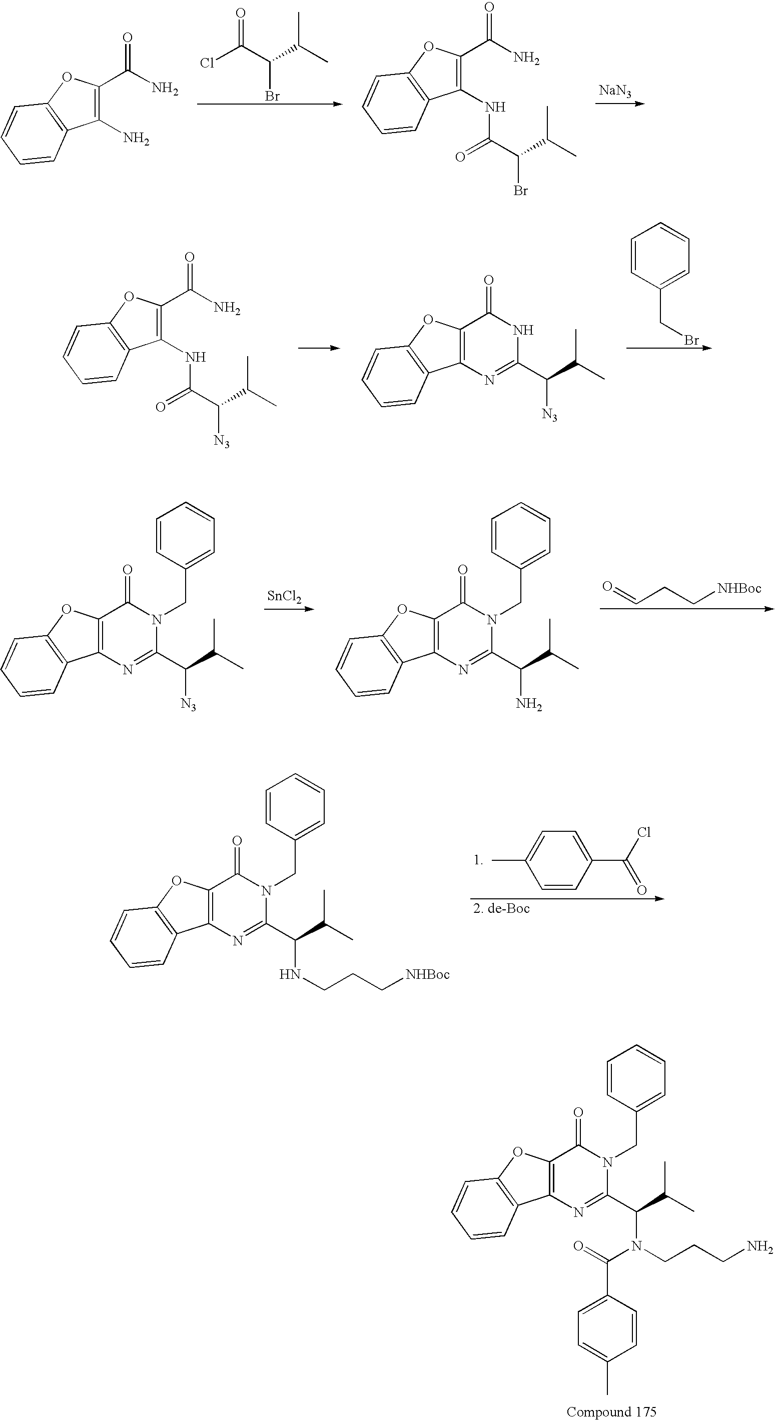

To a solution of 3-aminobenzofuran-2-carboxamide (2.0 g, 11.35 mmol) in dry DMF (16 mL) was added (S)-2-bromo-3-methylbutanoyl chloride (2.72 g, 13.64 mmol) dropwise at room temperature. After the addition, the reaction mixture was allowed to stir for 2 hr and then poured into 200 mL of water. The suspension was stirred at room temperature for 1 hr and the solid formed was collected by filtration. The collected solid was dried under high vacuum. The solid was dissolved in DMF (16 mL) followed by addition of NaN₃ (0.89 g, 13.64 mmol). The mixture was stirred at room temperature for 17 hr. The reaction mixture was poured into 200 mL water and stirred at room temperature for 1 hr. The precipitate was collected by filtration and dried under vacuum. The collected solid was dissolved in the solution of 2N NaOH (20 mL) and EtOH (10 mL). The resulting solution was heated to reflux for 4 hr. The reaction mixture was allowed to cool to room temperature and neutralized with 6N HCl solution to pH 7. The organic solvents were removed under vacuum. The resultant solid was collected by filtration and dried under high vacuum to afford 0.8 g of (R)-2-(1-azido-2-methylpropyl)benzofuro[3,2-d]pyrimidin-4(3H)-one. LC-MS (M+H): 284.1.

A mixture of (R)-2-(1-azido-2-methylpropyl)benzofuro[3,2-d]pyrimidin-4(3H)-one (0.3 g, 1.06 mmol) and cesium carbonate (0.7 g, 2.15 mmol) in 1,4-dioxane (10 mL) was stirred at room temperature for 30 min. Benzyl bromide (0.27 g, 1.59 mmol) was added. The mixture was heated at 100° C. for 2 h. It was then diluted with CH₂Cl₂ (50 mL) and washed with H₂O (20 mL). The organic layer was collected, dried over sodium sulfate, filtrated, and concentrated in vacuum. The crude material was purified by silica gel chromatography with 5% ethyl acetate/hexane to give 0.24 g of (R)-2-(1-azido-2-methylpropyl)-3-benzylbenzofuro[3,2-d]pyrimidin-4(3H)-one. LC-MS (M+H): 374.1.

The mixture of (R)-2-(1-azido-2-methylpropyl)-3 benzylbenzofuro[3,2-d]-pyrimidin-4(3H)-one (0.24 g, 0.64 mmol) and tin chloride (0.48 g, 2.55 mmol) in MeOH (10 mL) was stirred at room temperature for 1 hr. The solvent was removed by evaporation and the resulting mixture was diluted with dichloromethane (100 mL) and washed with 2N NaOH (30 mL). The organic layer was collected, dried over sodium sulfate, filtrated, and concentrated in vacuo. The crude solid was purified by silica gel chromatography with 50% ethyl acetate/hexane to give 0.21 g of (R)-2-(1-amino-2-methylpropyl)-3-benzyl-benzofuro[3,2-d]pyrimidin-4(3H)-one. LC-MS (M+H): 348.1.

To a solution of (R)-2-(1-amino-2-methylpropyl)-3-benzylbenzofuro[3,2-d]-pyrimidin-4(3H)-one (0.21 g, 0.603 mmol) in 10 mL of dichloromethane was added tert-butyl 3-oxopropylcarbamate (0.12 g, 0.69 mmol) followed by sodium triacetoxyborohydride (0.19 g, 0.90 mmol). The reaction mixture was stirred at room temperature overnight and diluted with dichloromethane (100 mL). The resulting solution was washed with 1.0 M ammonium hydroxide (20 mL). The organic layer was collected, dried over magnesium sulfate, filtrated, and concentrated in vacuo. The crude product was purified by silica gel chromatography with 20% ethyl acetate/hexane to give 0.21 g of (R)-tert-butyl 3-(1-(3-benzyl-4-oxo-3,4-dihydrobenzofuro[3,2-d]pyrimidin-2-yl)-2-methylpropylamino)propy-lcarbamate. LC-MS (M+H):505.0.

To a solution of (R)-tert-butyl 3-(1-(3-benzyl-4-oxo-3,4-dihydrobenzofuro[3,2-d]pyrimidin-2-yl)-2-methylpropylamino)propylcarbamate (0.21 g, 0.42 mmol) and triethylamine (0.18 mL, 1.26 mmol) in dichloromethane (5 mL) at 0° C. was added p-toluoyl chloride (0.083 mL, 0.63 mmol). The mixture was stirred at room temperature overnight and washed consecutively with saturated NaHCO₃ (10 mL) solution and water (20 mL). The organic layer was dried with magnesium sulfate, filtered, and evaporated under vacuum. The crude solid was purified by silica gel chromatography with 15% ethyl acetate/hexane to give 0.17 g of the Boc-protected compound 175. ¹H NMR (300 MHz, CDCl₃): δ 8.09 (d, J=7.8 Hz, 1H), 7.68 (dd, J=8.4, 2.1 Hz, 1H), 7.59 (td, J=6.9, 1.2 Hz, 1H), 7.45-7.40 (m, 3H), 7.32-7.17 (m, 7H), 6.27 (ABq, J=15.0 Hz, 1H), 5.79 (d, J=11.4 Hz, 1H), 5.32 (ABq, J=15.0 Hz, 1H), 3.87 (bs, 1H), 3.57-3.33 (m, 2H), 2.89-2.77 (m, 1H), 2.61-2.59 (m, 2H), 2.35 (s, 3H), 1.33 (m, 9H), 1.27-1.20 (m, 1H), 0.96 (d, J=6.6 Hz, 3H), 0.76-0.66 (m, 1H), 0.30 (d, J=6.6 Hz, 3H).

To the solution of the Boc-protected product (0.17 g, 0.27 mmol) in CH₂Cl₂ (3 mL) was added 4 N HCl in 1,4-dioxane (3 mL). The mixture was stirred at room temperature for 4 hr. The solvent was evaporated and the resulting solid was washed with ether and dried under high vacuum to give 0.1 g of the hydrochloride salt of compound 175. LC-MS (M+H): 522.7.

Alternatively, compound 175 was synthesized by the following procedure:

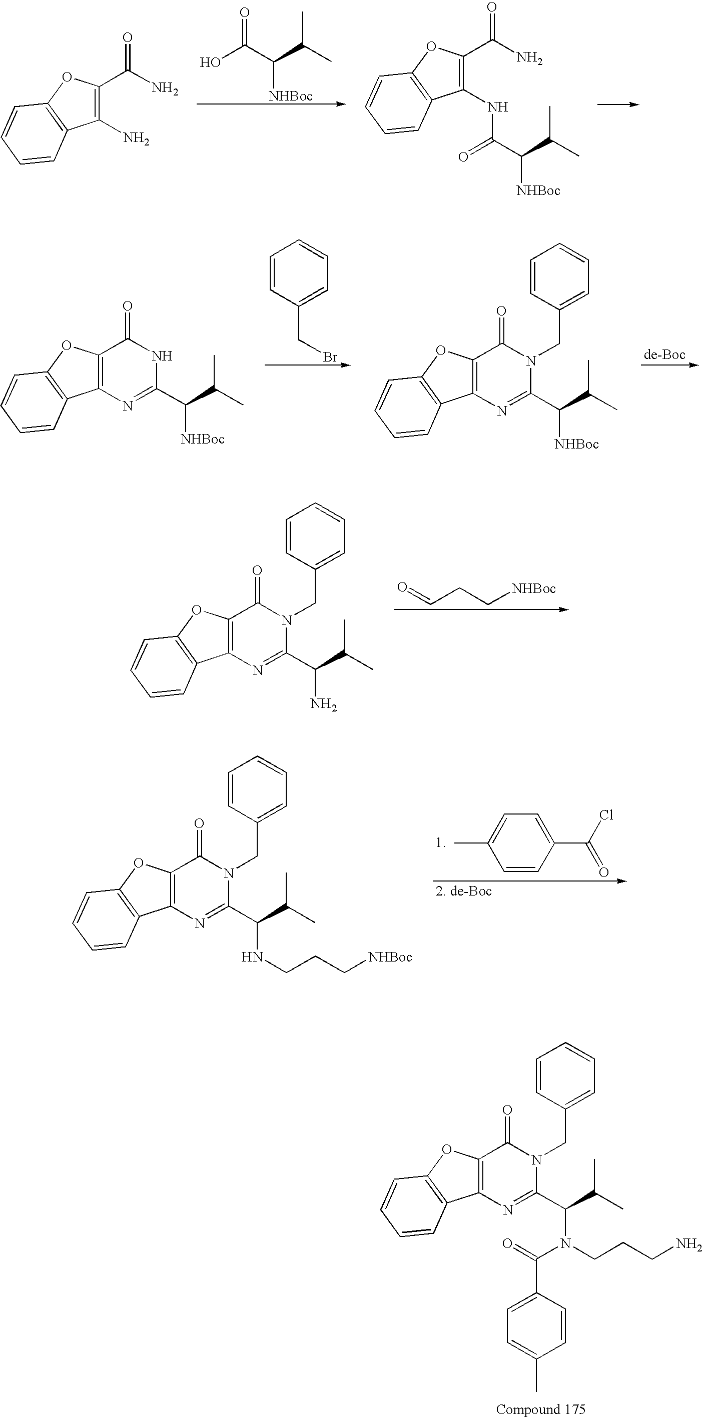

To a stirred solution of 3-aminobenzofuran-2-carboxamide (1.2 g, 6.81 mmol) in 41 mL of CH₂Cl₂ was added HATU (7.77 g, 20.43 mmol), N-methylmorpholine (4.6 mL, 34.1 mmol) and Boc-D-Valine (1.78 g, 8.17 mmol) at 0° C. and the mixture was stirred at room temperature for 5 days. The mixture was concentrated in vacuo and the crude solid was dissolved in dichloromethane. The dichloromethane layer was washed with NaHCO₃ aqueous solution, brine, dried over anhydrous MgSO₄, and concentrated in vacuo. The crude product was purified by silica gel chromatography with 30% ethyl acetate/hexane to afford 1.71 g of (R)-tert-butyl-1-(2-carbamoylbenzofuran-3-ylamino)-3-methyl-1-oxo-1-butan-2-ylcarbamate. ¹H NMR (300 MHz, CDCl₃): δ 0.97 (d, J=6.6 Hz, 3H), 1.04 (d, J=6.6 Hz, 3H), 1.45 (s, 9H), 4.37 (m, 1H), 5.26 (d, J=9.0 Hz, 1H), 7.23-7.28 (m, 1H), 7.37-3.45 (m, 2H), 8.41 (d, J=8.1 Hz, 1H), 10.27 (s, 1H). LC-MS (M+1): 376.2.

To a solution of (R)-tert-butyl-1-(2-carbamoylbenzofuran-3-ylamino)-3-methyl-1-oxobutan-2-ylcarbamate (1.71 g, 4.55 mmol) in 24 mL of EtOH was added 24 mL of 1N NaOH aqueous solution. The mixture was stirred at 50° C. for 24 hr. The solvent was removed by evaporation and the resulting residue was treated with 2N HCl aqueous solution at 0° C. till pH ˜2. The precipitate was collected by filtration and dried under reduced pressure to afford 0.67 g of (R)-tert-butyl 2-methyl-1-(4-oxo-3,4-dihydrobenzo-1-furo[3,2-d]pyrimidin-2-yl)propylcarbamate. LC-MS (M+H): 358.1.

A mixture of (R)-tert-butyl-2-methyl-1-(4-oxo-3,4-dihydrobenzofuro[3,2-d]-pyrimidin-2-yl)propylcarbamate (0.67 g, 1.87 mmol) and cesium carbonate (1.22 g, 3.74 mmol) in 9.4 mL of 1,4-dioxane was stirred at room temperature for 30 min. Benzyl bromide (0.33 g, 2.81 mmol) was added and the mixture was stirred at 110° C. for 30 min. 1,4-dioxane was removed under reduced pressure and the crude residue was dissolved in dichloromethane and washed with water. The dichloromethane layer was washed with brine, dried over MgSO₄, and concentrated in vacuo. The crude material was chromatographed on silica gel with 10% ethyl acetate/hexane to give 0.39 g of (R)-tert-butyl 1-(3-benzyl-4-oxo-3,4-dihydrobenzofuro[3,2-d]pyrimidin-2-yl)-2-methylpropyl-1-carbamate. LC-MS (M+H): 448.2.

To a stirred solution of (R)-tert-butyl 1-(3-benzyl-4-oxo-3,4-dihydrobenzofuro-1-[3,2-d]pyrimidin-2-yl)-2-methylpropylcarbamate (0.39 g, 0.86 mmol) in 8.6 mL of dichloromethane was added 4.3 mL of 4N HCl in dioxane solution at 0° C. The mixture was stirred at room temperature for 16 hr and the organic solvent was evaporated under reduced pressure. The precipitate was collected by filtration and dried under reduced pressure to give 0.28 g of (R)-2-(1-amino-2-methylpropyl)-3-benzylbenzofuro[3,2-d]-pyrimidin-4(3H)-one. ¹H NMR (300 MHz, CDCl₃): δ 0.96 (d, J=6.6 Hz, 3H), 1.02 (d, J=6.6 Hz, 3H), 2.06 (m, 1H), 4.53 (s, 1H), 5.20 (ABq, J=16.2 Hz, 1H), 5.44 (ABq, J=16.2 Hz, 1H), 6.59 (t, J=6.6 Hz, 1H), 7.01-7.25 (m, 2H), 7.29-7.47 (m, 5H), 7.92 (d, J=8.1 Hz, 1H), 9.47 (s, 1H), LC-MS (M+1): 348.1.

A mixture of (R)-2-(1-amino-2-methylpropyl)-3-benzylbenzofuro[3,2-d-pyrimi-1-din-4(3H)-one (0.16 g, 0.46 mmol) and tert-butyl 3-oxopropylcarbamate (0.55 g, 0.095 mmol) in 2.3 mL of dry dichloromethane was stirred at room temperature. Sodium triacetoxyborohydride (0.15 g, 0.69 mmol) was added into the mixture at 0° C. and the mixture was stirred at room temperature for 4 hr. The mixture was diluted with dichloromethane and washed with ammonium hydroxide solution. The organic layer was washed with brine, dried over MgSO₄, and concentrated in vacuo. The crude material was purified by silica gel chromatography with 25% ethyl acetate/hexane to give 0.07 g of (R)-tert-butyl 3-(1-(3-benzyl-4-oxo-3,4-dihydrobenzofuro[3,2-d]pyrimidin-2-yl)-2-methylpropylamino)propylcarbamate. LC-MS (M+H): 505.2.

To a solution of (R)-tert-butyl 3-(1-(3-benzyl-4-oxo-3,4-dihydrobenzofuro[3,2-d]-pyrimidin-2-yl)-2-methylpropylamino)propylcarbamate (0.15 g, 0.30 mmol) and triethylamine (0.07 mL, 0.90 mmol) in 1.5 mL of dry dichloromethane was added 4-methylbenzoyl chloride (0.08 mL, 0.60 mmol) at 0° C. The mixture was stirred at room temperature overnight and treated with saturated NaHCO₃ aqueous solution. The dichloromethane layer was washed with brine, dried over MgSO₄, and concentrated in vacuo. The crude material was chromatographed on silica gel with 20% ethyl acetate/hexane to afford 0.16 g of the Boc-protected compound 175. ¹H-NMR (CDCl₃, 300 Hz): δ0.36 (d, J=6.3 Hz, 3H), 0.71-0.77 (m, 1H), 1.01 (d, J=6.3 Hz, 3H), 1.39 (s, 9H), 2.42 (s, 3H), 2.65 (d, J=6.9 Hz, 2H), 2.84-2.92 (m, 1H), 3.40-3.62 (m, 2H), 3.84 (m, 1H), 5.62 (ABq, J=15.6 Hz, 1H), 5.84 (d, J=10.5 Hz, 1H), 6.33 (ABq, J=15.6 Hz, 1H), 7.38-7.39 (m, 6H), 7.46-7.51 (m, 3H), 7.66 (dd, J=8.1, 7,2 Hz, 1H), 7.75 (d, J=8.4, 1H), 8.15 (d, J=7.8 Hz, 1H), LC-MS (M+H): 623.0.

To a solution of Boc-protected compound 175 (0.16 g, 0.26 mmol) in 2.6 mL of methylene chloride was added 1.3 mL of 4N HCl in dioxane at 0° C. The mixture was stirred at room temperature for 5 hr and the organic solvent was removed by evaporation. The resulting residue washed with ether and dried under high vacuum to give 0.11 g of the hydrochloride salt of compound 175. LC-MS (M+H): 522.7.

### EXAMPLE 176

### Preparation of Compound 176

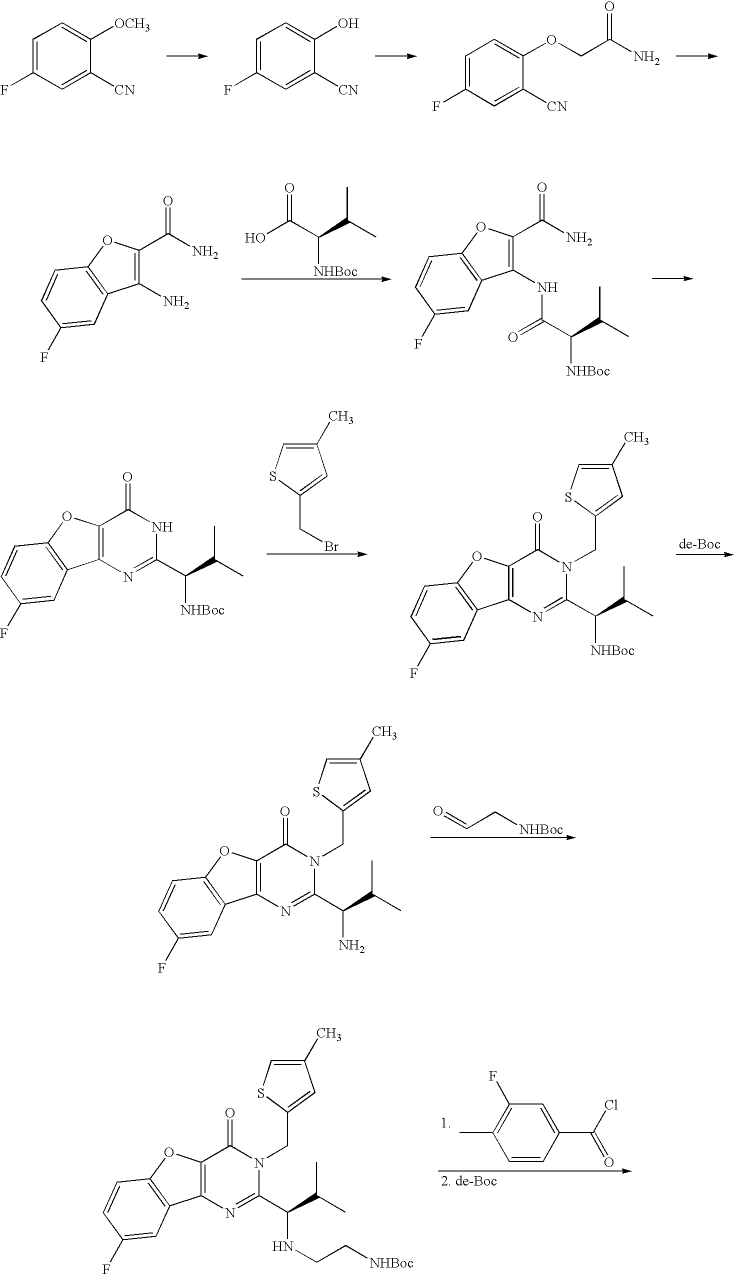

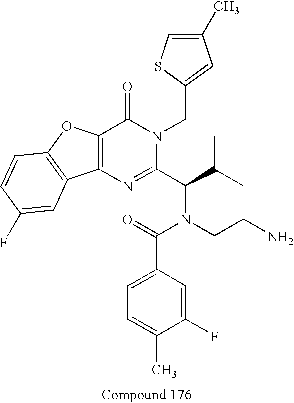

A mixture of 2-aminoethanethiol hydrochloride (8.1 g, 71.46 mmol) and sodium tert-butoxide (14.9 g, 154.83 mmol) in 120 mL of dry DMF was stirred at room temperature for 1 hr. 5-Fluoro-2-methoxybenzonitrile (9.0 g, 59.55 mmol) was added. After heated at 140° C. for 1 hr, the mixture was poured into 500 mL of ice water and acidified by 6N HCl aqueous solution to pH 2. The solution was extracted with ethyl acetate (2×1000 mL) and the combined organic layers were washed with water (5×500 mL) and dried over anhydrous MgSO₄ to give 7.3 g of the crude 5-fluoro-2-hydroxybenzonitrile. LC-MS (M+23): 160.0.

A mixture of 5-fluoro-2-hydroxybenzonitrile (7.3 g, 53.24 mmol) and potassium carbonate (22.1 g, 159.72 mmol) in 106 mL of dry DMF was stirred at 60° C. for 1 h. 2-Chloroacetamide (7.47 g, 79.86 mmol) was added. After heated at 80° C. for 3 h, the mixture was poured into 800 mL of water and stirred for 30 min. The precipitate was collected by filtration and dried under reduced pressure to give 8.8 g of 2-(2-cyano-4-fluorophenoxy)acetamide. LC-MS (M+1): 195.1.

To a stirred solution of 2-(2-cyano-4-fluorophenoxy)acetamide (8.8 g, 45.32 mmol) in 90 mL of EtOH was added NaOH (1.8 g, 45.32 mmol). After stirred at refluxing temperature for 1 hr, the mixture was evaporated and the residue was poured into 300 mL of water and acidified by 6N HCl aqueous solution to pH 2. The precipitate was collected by filtration and dried under reduced pressure to give 7.48 g of 3-amino-5-fluorobenzofuran-2-carboxamide. LC-MS (M+1): 195.0.

HATU (6.46 g, 17.0 mmol), N-methylmorpholine (3.52 g, 28.33 mmol) and Boc-D-valine (1.48 g, 6.80 mmol) were added to the solution of 3-amino-5-fluorobenzo-furan-carboxamide (1.1 g, 5.67 mmol) in 23 mL of CH₂Cl₂ at 0° C. After stirred at 50° C. for 5 days, the mixture was concentrated in vacuo and dissolved in dichloromethane. The dichloromethane layer was washed with NaHCO₃ aqueous solution, brine, dried over anhydrous MgSO₄, and concentrated in vacuo. The crude product was purified by silica gel chromatography with 50% ethyl acetate/hexane to give 0.45 g of (R)-tert-butyl 1-(2-carbamoyl-5-fluorobenzofuran-3-ylamino)-3-methyl-1-oxobutan-2-ylcarbamate. ¹H NMR (300 MHz, CDCl₃): δ 10.28 (s, 1H), 8.16 (dd, J=9.3 Hz, 2.7 Hz, 1H), 7.35 (dd, J=9.0 Hz, 4.2 Hz, 1H), 7.18 (ddd, J=8.9 Hz, 8.9 Hz, 2.7 Hz, 1H), 6.41 (s, 1H), 6.07 (s, 1H), 5.26 (d, J=8.1 Hz, 1H), 4.41 (m, 1H), 2.35-2.24 (m, 1H), 1.49 (s, 9H), 1.07 (d, J=6.6 Hz, 3H), 1.00 (d, J=6.9 Hz, 3H). LC-MS (M+1): 394.1.

To a solution of (R)-tert-butyl-1-(2-carbamoyl-5-fluorobenzofuran-3-ylamino)-3-methyl-1-oxobutan-2-ylcarbamate (0.71 g, 1.81 mmol) in 9.1 mL of EtOH was added 9.1 mL of 4 N LiOH aqueous solution. The mixture was stirred at 50° C. overnight. The solvent was removed by evaporation and the resulting residue was treated with 2 N HCl aqueous solution at 0° C. till pH ˜2. The precipitate was collected by filtration and dried under reduced pressure to give 0.41 g of (R)-tert-butyl-1-(8-fluoro-4-oxo-3,4-dihydrobenzo-furo[3,2-d]pyrimidin-2-yl)-2-methylpropylcarbamate. LC-MS (M+H): 376.1.

To a solution of (4-methylthiophen-2-yl)methanol (1.35 g, 10.51 mmol) in 53 mL of dichloromethane was added the solution of PBr₃ (1.48 mL, 15.77 mmol) in 7.4 mL of dichloromethane at 0° C. The mixture was stirred at room temperature for 2 hr and poured into ice water. The aqueous solution was treated with 1N NaOH to pH 7-8. The dichloromethane layer was washed with brine, dried over MgSO₄, and concentrated in vacuo to give 1.75 g of crude 2-(bromomethyl)-4-methylthiophene. ¹H-NMR (CDCl₃, 300 Hz): δ 2.21 (s, 3H), 4.69 (s, 2H), 6.88 (s, 1H), 6.92 (s, 1H).

A mixture of (R)-tert-butyl-1-(8-fluoro-4-oxo-3,4-dihydrobenzofuro[3,2-d]-pyrimidin-2-yl)-2-methylpropylcarbamate (0.41 g, 1.1 mmol) and cesium carbonate (0.71 g, 2.2 mmol) in 11 mL of 1,4-dioxane was stirred at room temperature for 30 min. 2-(Bromomethyl)-4-methylthiophene (0.31 g, 1.64 mmol) was added and the mixture was stirred at 100° C. for 1 hr. 1,4-Dioxane was removed under reduced pressure and the residue was dissolved in dichloromethane and washed with water. The dichloromethane layer was washed with brine, dried over MgSO₄, and concentrated in vacuo. The crude material was purified by silica gel chromatography with 10% ethyl acetate/hexane to give 0.30 g of (R)-tert-butyl-1-(8-fluoro-3-((4-methylthiophen-2-yl)methyl)-4-oxo-3,4-dihydrobenzofuro[3,2-d]pyrimidin-2-yl)-2-methylpropylcarbamate. LC-MS (M+H): 486.2.

(R)-tert-butyl 1-(8-fluoro-3-((4-methylthiophen-2-yl)methyl)-4-oxo-3,4-dihydro-benzofuro[3,2-d]pyrimidin-2-yl)-2-methylpropylcarbamate (0.30 g, 0.63 mmol) was dissolved in 6.3 mL of dichloromethane. 3.2 mL of 4 N HCl in dioxane solution was added at 0° C. The mixture was stirred at room temperature for 7 hr and the organic solvent was removed by evaporation. The residue was washed with saturated NaHCO₃ aqueous solution and extracted with dichloromethane. The dichloromethane layer was washed with brine, dried over MgSO₄, and concentrated in vacuo. The crude material was purified by silica gel chromatography with 50% ethyl acetate/hexane to give 0.17 g of (R)-2-(1-amino-2-methylpropyl)-8-fluoro-3-((4-methylthiophen-2-yl)methyl)benzofuro[3,2-d]pyrimidin-4(3H)-one. LC-MS (M+H): 386.0. ¹H-NMR (CDCl₃, 300 Hz): δ 0.94 (d, J=6.6 Hz, 3H), 1.05 (d, J=6.6 Hz, 3H), 2.17 (m, 1H), 2.20 (s, 3H), 3.98 (d, J=6.3 Hz, 1H), 5.33 (ABq, J=15.6 Hz, 1H), 5.88 (ABq, J=15.6 Hz, 1H), 6.81 (s, 1H), 6.88 (s, 1H), 7.32 (ddd, J₁=9.0 Hz, J₂=9.0 Hz, J₃=2.7 Hz, 1H), 7.62 (dd, J₁=9.2 Hz, J₂=4.1 Hz, 1H), 7.71 (dd, J₁=7.7 Hz, J₂=2.6 Hz, 1H).

A solution of (R)-2-(1-amino-2-methylpropyl)-8-fluoro-3-((4-methylthiophen-2-yl)-methyl)benzofuro[3,2-d]pyrimidin-4(3H)-one (0.17 g, 0.45 mmol) and tert-butyl N-(2-oxoethyl)carbamate (0.09 g, 0.54 mmol) in 2.2 mL of dry dichloromethane was stirred at room temperature. Sodium triacetoxyborohydride (0.14 g, 0.67 mmol) was added at 0° C. After stirred at room temperature for 1 hr, the mixture was diluted with dichloromethane and washed with ammonium hydroxide solution. The organic layer was washed with brine, dried over MgSO₄, and concentrated in vacuo. The crude material was purified by silica gel chromatography with 25% ethyl acetate/hexane to give 0.07 g of (R)-tert-butyl-2-(1-(8-fluoro-3-((4-methylthiophen-2-yl)methyl)-4-oxo-3,4-dihydrobenzofuro[3,2-d]pyrimidin-2-yl)-2-methylpropylamino)ethylcarbamate. LC-MS (M+H): 528.9.

To a solution of (R)-tert-butyl-2-(1-(8-fluoro-3-((4-methylthiophen-2-yl)methyl)-4-oxo-3,4-dihydrobenzofuro[3,2-d]pyrimidin-2-yl)-2-methylpropylamino)ethylcarbamate (0.07 g, 0.13 mmol) and triethylamine (0.05 mL, 0.40 mmol) in 1.3 mL of dry dichloromethane was added 3-fluoro-4-methylbenzoyl chloride (0.04 mL, 0.26 mmol) at 0° C. The mixture was stirred at room temperature overnight and washed with saturated NaHCO₃ aqueous solution. The organic layer was washed with brine, dried over MgSO₄, and concentrated in vacuo. The crude material was purified by silica gel chromatography with 25% ethyl acetate/hexane to give 68 mg of the Boc-protected compound 176. LC-MS (M+H): 665.0. ¹H-NMR (CDCl₃, 300 Hz): δ0.53 (d, J=6.0 Hz, 3H), 1.09 (d, J=6.6 Hz, 3H), 1.27 (s, 9H), 2.21 (s, 3H), 2.30 (s, 3H), 2.71 (m, 2H), 2.87 (m, 1H), 3.61 (m, 2H), 3.94 (m, 1H), 5.58 (ABq, J=15.4 Hz, 1H), 6.04 (d, J=10.8 Hz, 1H), 6.10 (ABq, J=15.4 Hz, 1H), 6.81 (s, 1H), 7.12 (m, 2H), 7.25 (m, 2H), 7.32 (ddd, J₁=10.7 Hz, J₂=10.7 Hz, J₃=2.6 Hz, 1H), 7.64 (dd, J₁=9.2 Hz, J₂=3.8 Hz, 1H), 7.75 (dd, J₁=9.8 Hz, J₂=4.1 Hz, 1H).

To a solution of the Boc-protected compound 176 (68 mg, 0.10 mmol) in 1 mL of dichloromethane was added 0.5 mL of 4 N HCl in dioxane at 0° C. The mixture was stirred at room temperature for 5 hr and the organic solvent was removed by evaporation. The resulting residue was washed with ether and dried under high vacuum to give 63 mg of the hydrochloride salt of compound 176. LC-MS (M+H): 565.0.

### EXAMPLES 177-221

### Preparation of Compounds 177-221

Compounds 177-208 were prepared in a manner similar to that described in Example 176. Their analytical data are provided below.

Compound 177: LC-MS (M+H): 595.1.

Compound 178: LC-MS (M+H): 558.2.

Compound 179: LC-MS (M+H): 547.2.

Compound 180: LC-MS (M+H): 541.2.

Compound 181: LC-MS (M+H): 567.2.

Compound 182: LC-MS (M+H): 567.1.

Compound 183: LC-MS (M+H): 611.1.

Compound 184: LC-MS (M+H): 561.2.

Compound 185: LC-MS (M+H): 517.2.

Compound 186: LC-MS (M+H): 535.1.

Compound 187: LC-MS (M+H): 545.2.

Compound 188: LC-MS (M+H): 563.2.

Compound 189: LC-MS (M+H): 551.1.

Compound 190: LC-MS (M+H): 569.1.

Compound 191: LC-MS (M+H): 527.2.

Compound 192: LC-MS (M+H): 545.2.

Compound 193: LC-MS (M+H): 547.1.

Compound 194: LC-MS (M+H): 533.1.

Compound 195: LC-MS (M+H): 551.1.

Compound 196: LC-MS (M+H): 517.2.

Compound 197: LC-MS (M+H): 535.2.

Compound 198: LC-MS (M+H): 537.1.

Compound 199: LC-MS (M+H): 533.1.

Compound 200: LC-MS (M+H): 551.1.

Compound 201: LC-MS (M+H): 599.0.

Compound 202: LC-MS (M+H): 599.0.

Compound 203: LC-MS (M+H): 611.1.

Compound 204: LC-MS (M+H): 617.0.

Compound 205: LC-MS (M+H): 591.1.

Compound 206: LC-MS (M+H): 563.2.

Compound 207: LC-MS (M+H): 581.2.

Compound 208: LC-MS (M+H): 536.2

Compound 209: LC-MS (M+H): 649.0

Compound 210: LC-MS (M+H): 677.0

Compound 211: LC-MS (M+H): 561.2

Compound 212: LC-MS (M+H): 595.1

Compound 213: LC-MS (M+H): 585.2

Compound 214: LC-MS (M+H): 603.1

Compound 215: LC-MS (M+H): 611.1

Compound 216: LC-MS (M+H): 663.0

Compound 217: LC-MS (M+H): 595.1

Compound 218: LC-MS (M+H): 615.1

Compound 219: LC-MS (M+H): 659.1

Compound 220: LC-MS (M+H): 609.1

Compound 221: LC-MS (M+H): 561.2

Compound 222: LC-MS (M+H): 545.1

Compound 223: LC-MS (M+H): 595.1

Compound 224: LC-MS (M+H): 548.8

Compound 225: LC-MS (M+H): 524.7

Compound 226: LC-MS (M+H): 530.6

### EXAMPLE 227

### KSP Enzyme Activity Assay

The efficacy of compounds 1-226 in inhibiting KSP enzymatic activity in the presence of microtubules was measured by an assay kit (PHOSFREE Phosphate Assay, Cytoskeleton, Denver, Colo.). This kit measures phosphate liberated from ATP by using a malachite green complex which specifically binds to phosphate ion. See Hackney et al, Methods Mol. Biol. 2001:164:65-71.

Recombinant HsEg5 motor domain (amino acids 1-368-6H is tag) was added to 50 μl of a reaction solution to reach a final concentration of 0.1 ng/μl. A buffer (9 mM PIPES, pH 7.5, 3 mM MgCl₂, 0.5 μM taxol) was added to the above solution. 5 μl of a test compound diluted in 0.1% DMSO was added to the wells of a 384-well plate, followed by addition of a substrate solution containing 2 mM ATP (final concentration 200 μM), BSA (final concentration 0.02%), and polymerized tubulin (final concentration 500 nM) in 10 μl of the buffer. 5 μl of the enzyme-containing buffer solution was then added to each well. The solution thus obtained was mixed and incubated with the test compound at 37° C. for 60 minutes. It was then mixed with an activator and incubated for an additional 15 minutes. The absorbance was read at 690 nm using a Multiskan (Thermo Electron Corporation, Waltham, Mass.). The absorbance data were graphed and the IC₅₀ values were calculated using Excel Fit.

All of compounds 1-226 exhibited IC₅₀ values less than 30 μM.

### EXAMPLE 228

### Cell Cycle Arrest Assay

A compound that specifically inhibits the activity of KSP can stop centrosome separation and result in the arrest of cancer cell cycle at the mitotic phase. The efficacy of a test compound to arrest cells as a KSP inhibitor was determined by flowcytometry analysis. Human Colo205 cells were grown in 6-well plates and treated the next day with the test compound for various time lengths. They were then scraped from plates using a rubber policeman, washed with PBS, and centrifuged at 1,000 rpm for 5 minutes. 1 ML of a buffer containing 10 μM of propidium iodide and 50 ug/ml of RNase A was used to re-suspend the cells. The cells were subsequently incubated in this buffer for 10 minutes at room temperature in the dark, passed through a filter to remove cell clumps, and observed under a Coulter Epics XL-MCL Training Modules flowcytometer.

Compounds 62, 66, 67, 81, 83, 84, 86, 168, and 177 were tested in this assay. All of them were found to cause a shift in the population of cells from a G0/G1 stage (2N DNA content) to a G2/M stage (4N DNA content).

### EXAMPLE 229

### Cytotoxicity Assay

The efficacy of a test compound in inhibiting tumor cell growth was analyzed using a MTS assay. The MTS assay is a calorimetric method for the determination of viable cells in proliferation by using a tetrazolium compound (3-(4,5-dimethylthiazol-2-yl)-5-(3-carboxymethylphenyl)-2-(4-sulfophenyl)-2H-tetrazolium, inner salt; MTS) and an electron-coupling agent (phenazine methosulfate; PMS). The MTS reagent is converted by dehydrogenase enzymes in metabolically active cells into a formazan that is soluble in a tissue culture medium.

In this assay, tumor cell suspensions (30 μl/well) were seeded into 384-well flat-bottomed tissue culture plates at an appropriate density overnight. A fresh medium (3 μl/well) containing varying concentrations of a test compound or vehicle was added to the culture followed by incubation for three days. At the end of the incubation, all plates were processed simultaneously as follows: The culture medium was aspirated, a MTS cell proliferation assay (Promega) solution (30 μl/well) was added, and the plates were incubated at 37° C. for about 2 hours. Absorbance (a parameter proportional to the cell mass) was measured at 490 nm using a Multiskan (Thermo Electron Corporation, Waltham, Mass.). Inhibition effects were expressed as corrected T/C values for each test compound according to the following equation:

T/C=(T−Blank)/(C−Blank)×100(%),

where T is the mean absorbance of the treated cells, C is the mean absorbance of the controls, and Blank is the mean absorbance of the cell free well.

Compounds 1-226 were tested in this assay. All of them exhibited IC₅₀ values less than 30 μM.

### EXAMPLE 230

### In Vivo Tumor Growth Inhibition Assay

The anti-cancer effect of compounds 36, 67, 81, 83, 84, 90, 94, 168, 176, 177, 179, 182, 183, and 189 was assessed in nude mice s.c. implanted tumor model.

The human colon cancer cell line (Colo205) obtained from the American Type Culture Collection (Rockville, Md.) was used for the xenograft model. The cell line was maintained in growth medium supplemented with L-glutamine, ribonucleosides, deoxyribonucleosides, 10% FCS, and the following antimicrobial agents: 100 IU/ml penicillin, 100 mg/ml streptomycin, and 0.25 mg/ml amphotericin B. Cultures were established in 75-cm² flasks (Costar, Cambridge, Mass.), maintained at 37° C. in a humidified atmosphere with 5% CO₂ in air, and subcultured every 4˜7 days with 0.25% trypsin in HBSS (Invitrogen Life Technologies, Carlsbad, Calif.). For in vivo tumor growth, cells (1×10⁷) suspended in 100 μl of PBS were inoculated in the flanks of 5-week-old female athymic BALB/cAnNCij-nu/nu mice (Charles River, Kanagawa, Japan). Seven days after inoculation (day 7), mice with tumors measuring 6-7 mm in diameter were randomly separated into 3 groups with five mice in each group. Compound 36 was dissolved in a vehicle (10% 40 mM sodium citrate, 5% Tween-80, 5% ethanol in saline) and intraperitoneally administered respectively to the groups of mice at the selected dose levels with the selected dosing regimen. A vehicle was administered to the third group of mice. Tumor size was measured twice weekly until day 30. Tumor volume (TV) was calculated as follows: volume=[length (mm)×width (mm)²]×0.52. The tumor growth for each group was expressed by the median values of the ratio between the tumor volume measured at a specific day to the initial tumor volume measured at the day of first dosing.

The results show significant tumor growth inhibition in the groups of mice treated with 40 mg/Kg and 30 mg/Kg of compound 36 once every 2 days for 5 times (q2dx5). Specifically, mice treated with 40 mg/Kg and 30 mg/Kg of compound 36 exhibited more than 83% tumor growth inhibition at day 10, more than 90% tumor growth inhibition at day 13, and more than 92% tumor growth inhibition at day 16. The results also indicate that a higher dose (40 mg/Kg) of compound 36 resulted in a stronger tumor inhibition than a lower dose (30 mg/Kg) of this compound.

Similar tumor growth inhibition results were obtained in the groups of mice treated with compounds 67, 84, 90, and 94. The mice treated with 10 mg/Kg of compound 67 once every 4 days for 3 times (q4dx3) exhibited more than 38% tumor growth inhibition at day 13. The mice treated with 60 mg/Kg of compound 84 once every 3 days for 3 times (q3dx3) exhibited more than 90% tumor growth inhibition at day 20. The mice treated with 40 mg/Kg of compound 90 once every 3 days for 3 times (q3dx3) exhibited more than 80% tumor growth inhibition at day 17. The mice treated with 40 mg/Kg of compound 94 once every 3 days for 3 times (q3dx3) exhibited more than 80% tumor growth inhibition at day 20.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of formula (I): 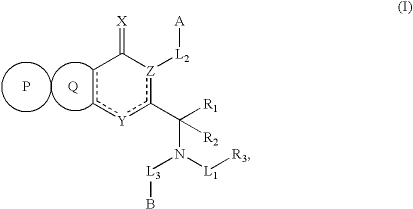
wherein 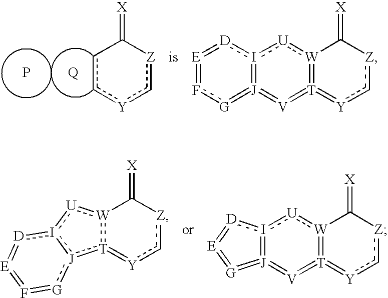
X is O or S;
each of D, E, F, G, I, J, T, U, V, W, Y, and Z, independently, is C, C(Rₐ₁), C(Rₐ₁Rₐ₂); N,N(Rₐ₁), O, or S; each of Rₐ₁ and Rₐ₂, independently, being H, halo, CN, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, heteroaryl, COOR, OCOR, N(RR′), C(O)—N(RR′), or N(R)—C(O)R′; in which each of R and R′, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, or heteroaryl;
each [symbol] independently, is a single bond or a double bond;
each of A and B, independently, is aryl or heteroaryl; in which aryl or heteroaryl is optionally substituted by 1, 2, or 3 substituents selected from the group consisting of halo, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, heteroaryl, CN, NO₂, OR_{b1}, SR_{b1}, C(O)R_{b1}, COOR_{b1}, O(C)OR_{b1}, C(O)—N(R_{b1}R_{b2}), N(R_{b1})—C(O)R_{b2}, NR_{b1}R_{b2}, S(O)R_{b1}, S(O)_{b2}R_{b1}, S(O)₂—NR_{b1}R_{b2}, NR_{b1}—S(O)₂R_{b2}, and C(NR_{b1})—NR_{b2}R_{b3}; each of R_{b1}, R_{b2}, and R_{b3}, independently, being H, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, or heteroaryl;
each of R₁, R₂, and R₃, independently, is H, halo, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, heteroaryl, CN, NO₂, OR_{c1}, SR_{c1}, C(O)R_{c1}, COOR_{c1}, O(C)OR_{c1}, C(O)—N(R_{c1}R_{c2}), N(R_{c1})—C(O)R_{c2}, NR_{c1}R_{c2}, S(O)R_{c1}, S(O)₂R_{c1}, S(O)₂—NR_{c1}R_{c2}, NR_{c1}—S(O)₂R_{c2}, or C(NR_{c1})—NR_{c2}R_{c3}; or R₁ and R₂, together with the carbon atom to which they are attached, are C₃-C₂₀ cycloalkyl, C₃-C₂₀ cycloalkenyl, C₃-C₂₀ heterocycloalkyl, or C₃-C₂₀ heterocycloalkenyl; each of R_{c1}, R_{c2} and R_{c3}, independently, being H, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, or heteroaryl;
each of L₁ and L₂, independently, is O, N(R_{d1}), C₁-C₁₀ alkylene, C₁-C₁₀ alkylcycloalkylene, C₂-C₁₀ alkenylene, C₂-C₁₀ alkynylene, or deleted; R_{d1} being H or C₁-C₁₀ alkyl; and
L₃ is CH₂, C(O), C(O)O, OC(O), SO, or SO₂.

2. The compound of claim 1, wherein R₁ is H; R₂ is H, halo, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, heteroaryl, CN, NO₂, OR_{c1}, SR_{c1}. C(O)R_{c1}, COOR_{c1}, O(C)OR_{c1}, C(O)—N(R_{c1}R_{c2}), N(R_{c1})—C(O)R_{c2}, NR_{c1}R_{c2}, S(O)R_{c1}, S(O)₂R_{c1}, S(O)₂—NR_{c1}R_{c2}, NR_{c1}—S(O)₂R_{c2} or C(NR_{c1})—NR_{c2}R_{c3}; and the compound has a configuration as shown in the following formula

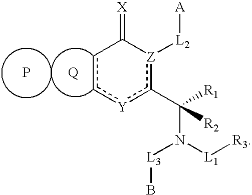

3. The compound of claim 2, wherein

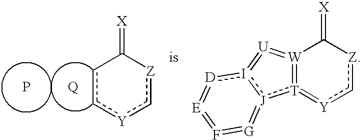

4. The compound of claim 3, wherein 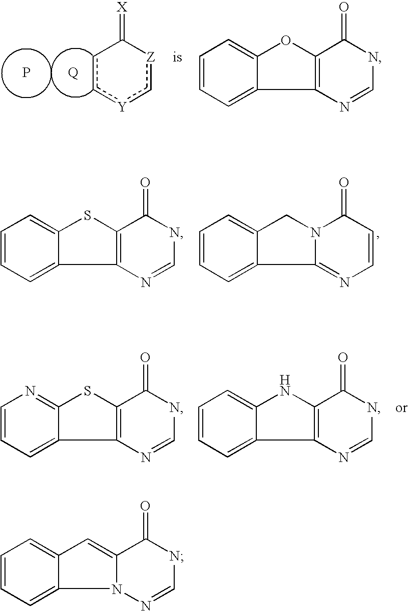
in which P is optionally substituted with F, Cl, Br, I, CN, COOR, OCOR, N(RR′), C(O)—N(RR′), N(R)—C(O)R′, or C₁-C₁₀ alkyl; the C₁-C₁₀ alkyl being optionally substituted with halo, C₂-C₁₀ alkenyl, or C₂-C₁₀ alkynyl.

5. The compound of claim 4, wherein L₁ is C₂-C₄ alkylene or ethylcyclobutylene optionally substituted with OH, halo, or N(Rₑ₁Rₑ₂); L₂ is methylene; and L₃ is C(O) or SO₂; in which each of Rₑ₁ and Rₑ₂, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, aryl, or heteroaryl.

6. The compound of claim 5, wherein R₁ is H; R₂ is ethyl, n-propyl, isopropyl, isobutyl, cyclopropyl, cyclobutyl, cyclopentyl, or C(O)—N(R_{c1}R_{c2}); or R₁ and R₂, together with the carbon atom to which they are attached, are C₃-C₂₀ cycloalkyl, C₃-C₂₀ cycloalkenyl, C₃-C₂₀ heterocycloalkyl, or C₃-C₂₀ heterocycloalkenyl; and R₃ is N(R_{c1})—C(O)R_{c2}, NR_{c1}R_{c2} or NR_{c1}—S(O)₂R_{c2}.

7. The compound of claim 6, wherein A is one of phenyl, pyridinyl, thienyl, furanyl, pyrrolyl, thiazolyl, isothiazolyl, oxazolyl, isooxazolyl, thiadiazolyl, oxadiazolyl, imidazolyl, and pyrazolyl, each of which is optionally substituted by 1, 2, or 3 substituents selected from the group consisting of F, Cl, Br, I, CN, NO₂, OR_{b1}, SR_{b1}, C₁-C₁₀ alkyl, C₂-C₁₀ alkenyl, C₂-C₁₀ alkynyl, C(O)R_{b1}, COOR_{b1}, O(C)OR_{b1}, C(O)—N(R_{b1}R_{b2}), N(R_{b1})—C(O)R_{b2}, NR_{b1}R_{b2}, S(O)R_{b1}, S(O)₂R_{b1}, S(O)₂—NR_{b1}R_{b2}, NR_{b1}—S(O)₂R_{b2}, and C(NR_{b1})—NR_{b2}R_{b3}.

8. The compound of claim 7, wherein B is one of phenyl, naphthyl, pyridinyl, thienyl, furanyl, pyrrolyl, thiazolyl, isothiazolyl, oxazolyl, isooxazolyl, thiadiazolyl, oxadiazolyl, imidazolyl, and pyrazolyl, each of which is optionally substituted by 1, 2, or 3 substituents selected from the group consisting of F, Cl, Br, I, CN, NO₂, OR_{b1}, SR_{b1}, C₁-C₁₀ alkyl, C₂-C₁₀ alkenyl, C₂-C₁₀ alkynyl, C(O)R_{b1}, COOR_{b1}, O(C)OR_{b1}, C(O)—N(R_{b1}R_{b2}), N(R_{b1})—C(O)R_{b2}, NR_{b1}R_{b2}, S(O)R_{b1}, S(O)₂R_{b1}, S(O)₂—NR_{b1}R_{b2}, NR_{b1}—S(O)₂R_{b2}, and C(NR_{b1})—NR_{b2}R_{b3}.

9. The compound of claim 2, wherein

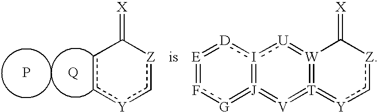

10. The compound of claim 9, wherein 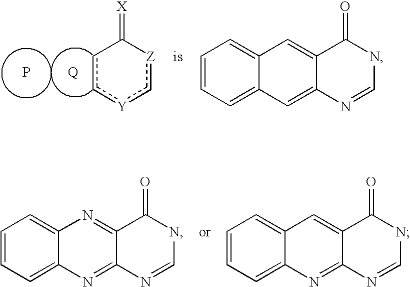
in which P is optionally substituted with Cl and Q is optionally substituted with Br, I, or CN.

11. The compound of claim 10, wherein L₁ is C₂-C₄ alkylene optionally substituted with halo, L₂ is methylene, and L₃ is CH₂ or C(O).

12. The compound of claim 11, wherein R₁ is H, R₂ is ethyl or isopropyl, R₃ is NH₂, A is phenyl or thienyl substituted with Cl, and B is phenyl substituted with CH₃.

13. The compound of claim 2, wherein

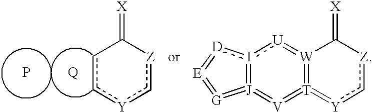

14. The compound of claim 13, wherein 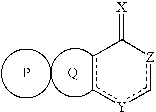
is 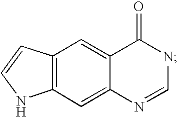
in which P is optionally substituted with Br.

15. The compound of claim 14, wherein L₁ is propylene, L₂ is methylene, and L₃ is C(O).

16. The compound of claim 15, wherein R₁ is H, R₂ is isopropyl, R₃ is NH₂, A is thienyl, and B is phenyl substituted with CH₃.

17. The compound of claim 1, wherein 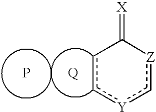
is selected from the group consisting of 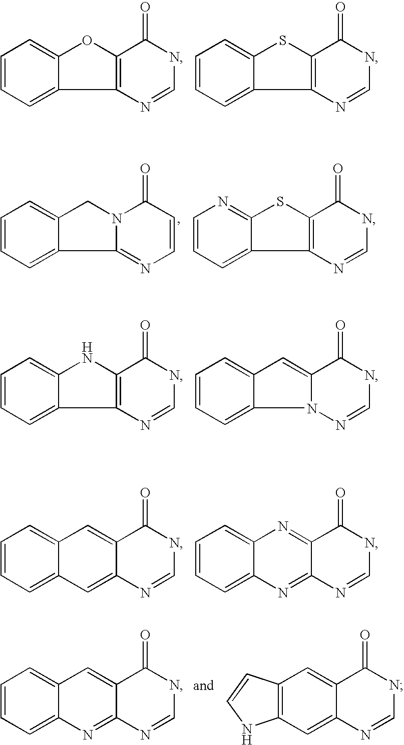
each of which is optionally substituted with F, Cl, Br, I, CN, COOR, OCOR, N(RR′), C(O)—N(RR′), N(R)—C(O)R′, or C₁-C₁₀ alkyl; the C₁-C₁₀ alkyl being optionally substituted with halo, C₂-C₁₀ alkenyl, or C₂-C₁₀ alkynyl.

18. The compound of claim 17, wherein A is one of phenyl, thienyl, and furanyl, each of which is optionally substituted with halo or C₁-C₁₀ alkyl; and B is phenyl optionally substituted with halo or C₁-C₁₀ alkyl.

19. The compound of claim 18, wherein L₁ is C₁-C₄ alkylene, L₂ is C₁-C₃ alkylene, and L₃ is C(O).

20. The compound of claim 1, wherein the compound is one of Compounds 1-226.

21. A pharmaceutical composition comprising a compound of claim 1 and a pharmaceutical acceptable carrier.

22. A method for treating a kinesin Eg5 protein-mediated disorder, comprising administering to a subject in need thereof an effective amount of the composition of claim 21, wherein the kinesin Eg5 protein-mediated disorder is cancer, hyperplasia, inflammation, immune disorder, restenosis, or cardiac hypertrophy.

23. The method of claim 22, wherein the kinesin Eg5 protein-mediated disorder is cancer.

24. The composition of claim 21, further comprising an anti-cancer agent selected from the group consisting of irinotecan, topotecan, gemcitabin, imatinib, trastuzuamb, 5-fluorouracil, leucovorin, carboplatin, cisplatin, docetaxel, paclitaxel, capecitabine, tezacitabine, cyclophosphamide, vinca alkaloid, anthracyclines, rituximab, and trastuzumab.

25. A method of claim 23, wherein the cancer is Hodgkin's disease, multiple myeloma, lymphoma, hematological neoplasm, leukemia, non-small-cell lung cancer, renal cell carcinoma, hepatocellular carcinoma, melanoma, prostate cancer, pancreatic cancer, gastric cancer, esophageal cancer, bladder cancer, breast cancer, head and neck cancer, ovarian cancer, or colorectal cancer.
